SEQUENCE LISTING (7 sequences)

SEQ ID NO: 1 - DNA, 3204 bp, Rift valley fever virus
atggcagggattgcaatgacagtccttccagccttagcagtttttgctttggcacctgttgtttttgctgaagacccccatctcagaaacagaccagggaaggggcacaactacattgacgggatgactcaggaggatgccacatgcaaacctgtgacatatgctggggcatgtagcagttttgatgtcttgcttgaaaagggaaaatttccccttttccagtcgtatgctcatcatagaactctactagaggcagttcacgacaccatcattgcaaaggctgatccacctagctgtgaccttctgagtgctcatgggaacccctgcatgaaagagaaactcgtgatgaagacacactgtccaaatgactaccagtcagctcatcacctcaacaatgacgggaaaatggcttcagtcaagtgccctcctaagtatgagctcactgaagactgcaacttttgtaggcagatgacaggtgctagcctgaagaaggggtcttatcctctccaagacttgttttgtcagtcaagtgaggatgatggatcaaaattaaaaacaaaaatgaaaggggtctgcgaagtgggggttcaagcactcaaaaagtgtgatggccaactcagcactgcacatgaggttgtgccctttgcagtgtttaagaactcaaagaaggtttatcttgataagcttgaccttaagactgaggagaatctgctaccagactcatttgtctgtttcgagcataagggacagtacaaaggaacaatggactctggtcagactaagagggagctcaaaagctttgatatctctcagtgccccaagattggaggacatggtagtaagaagtgcactggggacgcagcattttgctctgcttatgagtgcactgctcagtacgccaatgcctattgttcacatgctaatgggtcagggattgtgcagatacaagtatcaggggtctggaagaagcctttatgtgtagggtatgagagagtggttgtgaagagagaactctctgccaagcccatccagagagttgagccttgcacaacttgtataaccaaatgtgagcctcatggattggttgtccgatcaacagggttcaagatatcatcagcagttgcttgtgctagcggagtttgcgtcacaggatcgcagagtccttccaccgagattacactcaagtatccagggatatcccagtcttctgggggggacataggggttcacatggcacacgatgatcagtcagttagctccaaaatagtagctcactgccctccccaggacccgtgcttagtgcatgactgcatagtgtgtgctcatggcctgataaattaccagtgtcacactgctctcagtgcctttgttgttgtgtttgtattcagttctattgcaataatttgtttagctattctttatagggtgcttaagtgcctgaagattgccccaaggaaagttctgaatccactaatgtggatcacagccttcatcagatggatatataagaagatggttgccagagtggcagacaacattaatcaagtgaacagggaaataggatggatggaaggaggtcagttggttctagggaaccctgcccctattcctcgtcatgccccaatcccacgttatagcacatacctgatgttattattgattgtctcatatgcatcagcatgttcagaactgattcaggcaagctccagaatcaccacttgctctacagagggtgttaacaccaagtgtagactgtctggcacagcattgatcagagcagggtcagttggggcagaggcttgtttgatgttgaagggggtcaaggaagatcaaaccaagttcttaaagttaaaaactgtctcaagtgagctatcatgcagggagggccagagttattggactgggtcctttagccctaaatgtttgagctcaaggagatgccaccttgtcggggaatgccatgtgaataggtgtctgtcttggagggacaatgaaacttcagcagagttttcatttgttggggaaagcacgaccatgcgagagaataagtgttttgagcaatgtggaggatgggggtgtgggtgtttcaatgtgaacccatcttgcttatttgtgcacacgtatctgcagtcagttagaaaagaggcccttagagtttttaactgtatcgactgggtgcataaactcactctagagatcacagactttgatggctctgtttcaacaatagacttgggagcatcatctagccgtttcacaaactggggttcagttagcctctcactggacgcagagggcatttcaggctcaaatagcttttctttcattgagagcccaggcaaagggtatgcaattgttgatgagccattctcagaaattcctcggcaagggttcttgggggagatcaggtgcaattcagagtcctcagtcctgagtgctcatgaatcatgccttagggcaccaaaccttatctcatacaagcccatgatagatcaattggagtgcacaacaaatctgattgatccctttgttgtctttgagaggggttctctgccacagacaaggaatgacaaaacctttgcagcttcaaaaggaaatagaggtgttcaagctttctctaagggctctgtacaagctgatctaactctgatgtttgacaattttgaggtggactttgtgggagcagccgtatcttgtgatgccgccttcttaaatttgacaggttgctattcttgcaatgcaggggccagggtctgcctgtctatcacatccacaggaactggatctctctctgcccacaataaggatgggtctctgcatatagtccttccatcagagaatggaacaaaagaccagtgtcagatactacacttcactgtgcctgaagtagaggaggagtttatgtactcttgtgatggagatgagcggcctctgttggtgaaggggaccctgatagccattgatccatttgatgataggcgggaagcagggggggaatcaacagttgtgaatccaaaatctggatcttggaatttctttgactggttttctggactcatgagttggtttggagggcctcttaaaactatactcctcatttgcctgtatgttgcattatcaattgggctctttttcctccttatatatcttggaggaacaggcctctctaaaatgtggcttgctgccactaagaaggcctcatag

SEQ ID NO: 2 - DNA, 3204 bp, Artificial Sequence
  Synthetic
atggccggcattgctatgacagtgctgcctgccctggccgtgttcgctctggcccctgtggtgttcgctgaagacccccatctcagaaacagacccggaaagggccacaactacattgacggaatgacacaggaggatgccacatgcaaacctgtgacatatgctggcgcctgtagcagcttcgatgtgctgctcgaaaagggaaaattccccctcttccagtcctatgctcatcatagaaccctgctggaggctgtgcacgacaccatcattgccaaggctgatcctcctagctgtgaccttctcagcgctcatggaaacccctgcatgaaagagaaactcgtgatgaagacacactgtcccaatgactaccagtccgcgcatcacctcaacaatgacggcaaaatggcttccgtgaagtgccctcctaagtatgagctcacagaagactgcaacttctgtaggcagatgacaggcgctagcctgaagaagggctcgtatcctctccaagacctgttctgtcagtccagcgaggatgatggatccaaactgaaaacaaaaatgaaaggcgtgtgcgaagtgggagtgcaagccctcaaaaagtgtgatggccaactcagcaccgctcatgaggtggtgcccttcgccgtgttcaagaactccaagaaggtgtatctggataagctggacctcaagaccgaggagaatctgctccctgactccttcgtgtgtttcgagcataagggacagtacaaaggaacaatggactccggacagacaaagagggagctcaaaagcttcgatatctcccagtgccccaagattggaggacatggaagcaagaagtgcacaggcgacgcagcattctgctccgcttatgagtgcaccgctcagtacgccaatgcctattgttcccatgctaatggctccggaattgtgcagatccaagtgtccggcgtgtggaagaagcctctctgtgtgggctatgagagagtggtggtgaagagagaactctccgccaagcccatccagagagtggagccttgcacaacctgtattaccaaatgtgagcctcatggactggtggtcagatccacaggcttcaagatttcctccgccgtggcttgtgctagcggagtgtgcgtgacaggatcccagagcccttccaccgagattacactcaagtatcctggcatttcccagtcctccggcggagacatcggcgtgcacatggcccacgatgatcagtccgtgagctccaaaattgtggctcactgccctccccaggacccttgcctcgtgcatgactgcattgtgtgtgctcatggcctgatcaattaccagtgtcacacagctctcagcgccttcgtggtggtgttcgtgttcagctccattgctatcatttgtctcgctattctgtatagagtgctgaagtgcctgaagattgcccctagaaaagtgctgaatcccctgatgtggatcacagccttcatcagatggatttataagaagatggtcgccagagtggccgacaacattaatcaagtgaacagggaaatcggatggatggaaggaggccagctggtgctcggcaaccctgcccctattcctagacatgcccccatccccaggtatagcacatacctcatgctgctcctgattgtgtcctatgcctccgcttgttccgaactgattcaggcaagctccagaatcaccacatgctccacagagggcgtgaacaccaagtgtagactgtccggcacagccctgatcagagctggctccgtgggagctgaggcttgtctgatgctgaagggagtgaaggaagatcaaaccaagttcctcaagctgaagacagtgtccagcgagctctcctgtagggagggccagagctattggacaggatccttcagccctaaatgtctgagctccaggagatgccacctcgtgggcgaatgccatgtgaataggtgtctgtcctggagggacaatgaaacctccgctgagttctccttcgtgggcgaaagcacaaccatgagagagaataagtgtttcgagcaatgtggaggatggggctgtggatgtttcaatgtgaacccttcctgcctgttcgtgcacacctatctgcagtccgtgagaaaagaggccctcagagtgttcaactgtatcgactgggtgcataaactcacactggagatcacagacttcgatggctccgtgtccacaatcgacctgggagcttcctccagcagattcacaaactggggatccgtgagcctctccctggacgccgagggcatttccggctccaatagcttctccttcattgagagccctggcaaaggatatgctattgtggatgagcctttctccgaaattcctaggcaaggattcctgggcgagatcaggtgcaattccgagtcctccgtgctgagcgctcatgaatcctgcctgagggctcccaacctcatctcctacaagcccatgattgatcaactggagtgcacaacaaatctgattgatcccttcgtggtgttcgagaggggctccctgccccagacaaggaatgacaaaaccttcgctgcttccaaaggaaatagaggcgtgcaagctttctccaagggctccgtgcaagctgatctgacactgatgttcgacaatttcgaggtggacttcgtgggagctgccgtgtcctgtgatgccgccttcctcaatctgacaggctgttattcctgcaatgcaggcgccagggtgtgcctgtccatcacatccacaggaacaggatccctctccgcccacaataaggatggatccctgcatatcgtgctcccttccgagaatggaacaaaagaccagtgtcagattctccacttcacagtgcctgaagtggaggaggagttcatgtactcctgtgatggagatgagaggcctctgctggtgaagggaaccctgattgccattgatcccttcgatgataggagggaagctggcggagaatccacagtggtgaatcccaaatccggatcctggaatttcttcgactggttctccggactcatgagctggttcggaggccctctcaaaacaattctcctcatttgcctgtatgtggctctctccattggcctcttcttcctcctgatttatctcggaggaacaggcctctccaaaatgtggctcgctgccacaaagaaggcctcctag

SEQ ID NO: 3 - DNA, 3228 bp, Artificial Sequence
  Synthetic
aatatagaattcgccaccatggccggcattgctatgacagtgctgcctgccctggccgtgttcgctctggcccctgtggtgttcgctgaagacccccatctcagaaacagacccggaaagggccacaactacattgacggaatgacacaggaggatgccacatgcaaacctgtgacatatgctggcgcctgtagcagcttcgatgtgctgctcgaaaagggaaaattccccctcttccagtcctatgctcatcatagaaccctgctggaggctgtgcacgacaccatcattgccaaggctgatcctcctagctgtgaccttctcagcgctcatggaaacccctgcatgaaagagaaactcgtgatgaagacacactgtcccaatgactaccagtccgcgcatcacctcaacaatgacggcaaaatggcttccgtgaagtgccctcctaagtatgagctcacagaagactgcaacttctgtaggcagatgacaggcgctagcctgaagaagggctcgtatcctctccaagacctgttctgtcagtccagcgaggatgatggatccaaactgaaaacaaaaatgaaaggcgtgtgcgaagtgggagtgcaagccctcaaaaagtgtgatggccaactcagcaccgctcatgaggtggtgcccttcgccgtgttcaagaactccaagaaggtgtatctggataagctggacctcaagaccgaggagaatctgctccctgactccttcgtgtgtttcgagcataagggacagtacaaaggaacaatggactccggacagacaaagagggagctcaaaagcttcgatatctcccagtgccccaagattggaggacatggaagcaagaagtgcacaggcgacgcagcattctgctccgcttatgagtgcaccgctcagtacgccaatgcctattgttcccatgctaatggctccggaattgtgcagatccaagtgtccggcgtgtggaagaagcctctctgtgtgggctatgagagagtggtggtgaagagagaactctccgccaagcccatccagagagtggagccttgcacaacctgtattaccaaatgtgagcctcatggactggtggtcagatccacaggcttcaagatttcctccgccgtggcttgtgctagcggagtgtgcgtgacaggatcccagagcccttccaccgagattacactcaagtatcctggcatttcccagtcctccggcggagacatcggcgtgcacatggcccacgatgatcagtccgtgagctccaaaattgtggctcactgccctccccaggacccttgcctcgtgcatgactgcattgtgtgtgctcatggcctgatcaattaccagtgtcacacagctctcagcgccttcgtggtggtgttcgtgttcagctccattgctatcatttgtctcgctattctgtatagagtgctgaagtgcctgaagattgcccctagaaaagtgctgaatcccctgatgtggatcacagccttcatcagatggatttataagaagatggtcgccagagtggccgacaacattaatcaagtgaacagggaaatcggatggatggaaggaggccagctggtgctcggcaaccctgcccctattcctagacatgcccccatccccaggtatagcacatacctcatgctgctcctgattgtgtcctatgcctccgcttgttccgaactgattcaggcaagctccagaatcaccacatgctccacagagggcgtgaacaccaagtgtagactgtccggcacagccctgatcagagctggctccgtgggagctgaggcttgtctgatgctgaagggagtgaaggaagatcaaaccaagttcctcaagctgaagacagtgtccagcgagctctcctgtagggagggccagagctattggacaggatccttcagccctaaatgtctgagctccaggagatgccacctcgtgggcgaatgccatgtgaataggtgtctgtcctggagggacaatgaaacctccgctgagttctccttcgtgggcgaaagcacaaccatgagagagaataagtgtttcgagcaatgtggaggatggggctgtggatgtttcaatgtgaacccttcctgcctgttcgtgcacacctatctgcagtccgtgagaaaagaggccctcagagtgttcaactgtatcgactgggtgcataaactcacactggagatcacagacttcgatggctccgtgtccacaatcgacctgggagcttcctccagcagattcacaaactggggatccgtgagcctctccctggacgccgagggcatttccggctccaatagcttctccttcattgagagccctggcaaaggatatgctattgtggatgagcctttctccgaaattcctaggcaaggattcctgggcgagatcaggtgcaattccgagtcctccgtgctgagcgctcatgaatcctgcctgagggctcccaacctcatctcctacaagcccatgattgatcaactggagtgcacaacaaatctgattgatcccttcgtggtgttcgagaggggctccctgccccagacaaggaatgacaaaaccttcgctgcttccaaaggaaatagaggcgtgcaagctttctccaagggctccgtgcaagctgatctgacactgatgttcgacaatttcgaggtggacttcgtgggagctgccgtgtcctgtgatgccgccttcctcaatctgacaggctgttattcctgcaatgcaggcgccagggtgtgcctgtccatcacatccacaggaacaggatccctctccgcccacaataaggatggatccctgcatatcgtgctcccttccgagaatggaacaaaagaccagtgtcagattctccacttcacagtgcctgaagtggaggaggagttcatgtactcctgtgatggagatgagaggcctctgctggtgaagggaaccctgattgccattgatcccttcgatgataggagggaagctggcggagaatccacagtggtgaatcccaaatccggatcctggaatttcttcgactggttctccggactcatgagctggttcggaggccctctcaaaacaattctcctcatttgcctgtatgtggctctctccattggcctcttcttcctcctgatttatctcggaggaacaggcctctccaaaatgtggctcgctgccacaaagaaggcctcctagctcgag

SEQ ID NO: 4 - DNA, 1476 bp, Lassa virus
atgggacaaatagtgacattcttccaggaagtgcctcatgtaatagaagaggtgatgaacattgttctcattgcactgtctgtactagcagtgctgaaaggtctgtacaattttgcaacgtgtggccttgttggtttggtcactttcctcctgttgtgtggtaggtcttgcacaaccagtctttataaaggggtttatgagcttcagactctggaactaaacatggagacactcaatatgaccatgcctctctcctgcacaaagaacaacagtcatcattatataatggtgggcaatgagacaggactagaactgaccttgaccaacacgagcattattaatcacaaattttgcaatctgtctgatgcccacaaaaagaacctctatgaccacgctcttatgagcataatctcaactttccacttgtccatccccaacttcaatcagtatgaggcaatgagctgcgattttaatgggggaaagattagtgtgcagtacaacctgagtcacagctatgctggggatgcagccaaccattgtggtactgttgcaaatggtgtgttacagacttttatgaggatggcttggggtgggagctacattgctcttgactcaggccgtggcaactgggactgtattatgactagttatcaatatctgataatccaaaatacaacctgggaagatcactgccaattctcgagaccatctcccatcggttatctcgggctcctctcacaaaggactagagatatttatattagtagaagattgctaggcacattcacatggacactgtcagattctgaaggtaaagacacaccagggggatattgtctgaccaggtggatgctaattgaggctgaactaaaatgcttcgggaacacagctgtggcaaaatgtaatgagaagcatgatgaggaattttgtgacatgctgaggctgtttgacttcaacaaacaagccattcaaaggttgaaagctgaagcacaaatgagcattcagttgatcaacaaagcagtaaatgctttgataaatgaccaacttataatgaagaaccatctacgggacatcatgggaattccatactgtaattacagcaagtattggtacctcaaccacacaactactgggagaacatcactgcccaaatgttggcttgtatcaaatggttcatacttgaacgagacccacttttctgatgatattgaacaacaagctgacaatatgatcactgagatgttacagaaggagtatatggagaggcaggggaagacaccattgggtctagttgacctctttgtgttcagtacaagtttctatcttattagcatcttccttcacctagtcaaaataccaactcataggcatattgtaggcaagtcgtgtcccaaacctcacagattgaatcatatgggcatttgttcctgtggactctacaaacagcctggtgtgcctgtgaaatggaagagatga

SEQ ID NO: 5 - DNA, 1476 bp, Artificial Sequence
  Synthetic
atgggtcagattgtgacattcttccaggaagtgcctcatgtgattgaagaggtgatgaacattgtgctcattgccctgtccgtgctcgccgtgctgaaaggtctgtacaattttgccacctgtggcctggtgggtctggtgacattcctcctgctgtgtggtaggtcctgcacaaccagcctctacaaaggtgtgtacgagctccagacactggaactgaacatggagacactcaatatgaccatgcctctctcctgcacaaagaacaacagccatcattacatcatggtgggcaatgagacaggcctggaactgaccctgaccaacacaagcattattaatcacaaattttgcaatctgtccgatgcccacaaaaagaacctctacgaccacgccctcatgagcattatctccaccttccacctgtccatccccaacttcaatcagtacgaggccatgagctgcgattttaatggcggtaagattagcgtgcagtacaacctgagccacagctacgccggcgatgccgccaaccattgtggtaccgtggccaatggtgtgctgcagacctttatgaggatggcctggggtggtagctacattgccctcgactccggcaggggcaactgggactgtattatgaccagctaccagtacctgattatccagaatacaacctgggaagatcactgccagttctccagaccatcccccatcggttacctcggtctcctctcccagaggaccagagatatttacattagcagaagactgctcggcacattcacatggacactgtccgattccgaaggtaaagacacaccaggtggctactgtctgaccaggtggatgctcattgaggccgaactcaaatgcttcggtaacacagccgtggccaaatgtaatgagaagcatgatgaggaattttgtgacatgctgaggctgtttgacttcaacaaacaggccattcagaggctgaaagccgaagcccagatgagcattcagctgatcaacaaagccgtgaacgccctgattaatgaccagctcatcatgaagaaccatctgagggacatcatgggcattccatactgtaattacagcaagtactggtacctcaaccacacaaccacaggtagaacatccctgcccaaatgttggctcgtgtccaatggttcctacctgaacgagacccacttttccgatgatattgaacagcaggccgacaatatgatcaccgagatgctgcagaaggagtacatggagaggcagggtaagacaccactgggtctggtggacctctttgtgttcagcacaagcttctacctcattagcatcttcctccacctggtgaaaattccaacacataggcatattgtgggcaagtcctgtcccaaacctcacagactgaatcatatgggcatttgttcctgtggtctctacaaacagcctggtgtgcctgtgaaatggaagagatga

SEQ ID NO: 6 - protein, 1067 aa, Rift valley fever virus
MAGIAMTVLPALAVFALAPVVFAEDPHLRNRPGKGHNYIDGMTQEDATCKPVTYAGACSSFDVLLEKGKFPLFQSYAHHRTLLEAVHDTIIAKADPPSCDLLSAHGNPCMKEKLVMKTHCPNDYQSAHHLNNDGKMASVKCPPKYELTEDCNFCRQMTGASLKKGSYPLQDLFCQSSEDDGSKLKTKMKGVCEVGVQALKKCDGQLSTAHEVVPFAVFKNSKKVYLDKLDLKTEENLLPDSFVCFEHKGQYKGTMDSGQTKRELKSFDISQCPKIGGHGSKKCTGDAAFCSAYECTAQYANAYCSHANGSGIVQIQVSGVWKKPLCVGYERVVVKRELSAKPIQRVEPCTTCITKCEPHGLVVRSTGFKISSAVACASGVCVTGSQSPSTEITLKYPGISQSSGGDIGVHMAHDDQSVSSKIVAHCPPQDPCLVHDCIVCAHGLINYQCHTALSAFVVVFVFSSIAIICLAILYRVLKCLKIAPRKVLNPLMWITAFIRWIYKKMVARVADNINQVNREIGWMEGGQLVLGNPAPIPRHAPIPRYSTYLMLLLIVSYASACSELIQASSRITTCSTEGVNTKCRLSGTALIRAGSVGAEACLMLKGVKEDQTKFLKLKTVSSELSCREGQSYWTGSFSPKCLSSRRCHLVGECHVNRCLSWRDNETSAEFSFVGESTTMRENKCFEQCGGWGCGCFNVNPSCLFVHTYLQSVRKEALRVFNCIDWVHKLTLEITDFDGSVSTIDLGASSSRFTNWGSVSLSLDAEGISGSNSFSFIESPGKGYAIVDEPFSEIPRQGFLGEIRCNSESSVLSAHESCLRAPNLISYKPMIDQLECTTNLIDPFVVFERGSLPQTRNDKTFAASKGNRGVQAFSKGSVQADLTLMFDNFEVDFVGAAVSCDAAFLNLTGCYSCNAGARVCLSITSTGTGSLSAHNKDGSLHIVLPSENGTKDQCQILHFTVPEVEEEFMYSCDGDERPLLVKGTLIAIDPFDDRREAGGESTVVNPKSGSWNFFDWFSGLMSWFGGPLKTILLICLYVALSIGLFFLLIYLGGTGLSKMWLAATKKAS

SEQ ID NO: 7 - protein, 1067 aa, Artificial Sequence; identical to SEQ ID NO: 6
  Synthetic

(19) United States
(12) Patent Application Publication
(10) Pub. No.: US 2009/0123494 A1
(43) Pub. Date: May 14, 2009

(54) MOMLV-BASED PSEUDOVIRION PACKAGING CELL LINE

(76) Inventors: William Staplin, Des Moines, IA (US); Robert Mandell, Collins, IA (US); Ramon Flick, Polk City, IA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/184,206

(22) Filed: Jul. 31, 2008

Related U.S. Application Data
(60) Provisional application No. 60/953,111, filed on Jul. 31, 2007.

(51) Int. Cl. A61K 39/12; C12N 7/04; C07H 21/04; C12N 15/63

(52) U.S. Cl. 424/205.1; 435/236; 536/23.1; 435/320.1

(57) ABSTRACT

The present invention discloses Moloney murine leukemia virus (MoMLV)-based viral packaging cell line for the production of anti-viral vaccines. The invention also includes methods of making, administering and formulating pseudovirions and replicon deficient viral particles of the invention and methods of inducing immunity.

### CROSS-REFERENCE TO RELATED APPLICATIONS

This application claims priority to U.S. Provisional Application No. 60/953,111, filed Jul. 31, 2007, which is herein incorporated by reference in its entirety.

### FIELD OF INVENTION

This invention relates primarily to Moloney murine leukemia virus (MoMLV) packaging cell lines capable of expressing heterologous viral glycoproteins and producing pseudotyped MoMLV viral particles, including replicon-deficient pseudotyped MoMLV viral particles. The replicon-deficient viral particles of the invention can be used, for instance, in the development of vaccines.

### BACKGROUND

Phenotypic mixing is a common occurrence in cells infected with two or more related and even unrelated enveloped viruses (Závada, “The Pseudotypic Paradox.” J. Gen. Virol. 63:15-24). In most instances, phenotypic mixing of viruses only occurs for the envelope glycoproteins. Based on this natural phenomenon, pseudovirus packaging cell line systems have been developed for the deliberate and systematic modification of the natural tropism of a large variety of viruses and virus-based vector systems.

Pseudovirus packaging cell line systems can be used, for instance, to enable researchers to better study viral protein interactions with receptors, viral entry and immunogenicity. Because most pseudoviruses are non-pathogenic and can be handled at Biosafety Level (BSL) 2, the ability to generate pseudotyped virions is advantageous in the study of high risk pathogenic viruses such as those categorized as BSL-3 and BSL-4 pathogens. Viruses that are high risk pathogens (e.g. BSL-4), include, but are not limited to, filoviruses (e.g., Ebola and Marburg), arenaviruses (e.g., Lassa virus) and bunyaviruses (e.g., Crimean Congo hemorrhagic fever virus and Rift Valley fever virus).

Previous attempts have been made to express glycoproteins from BSL-4 viruses such as filovirus and arenavirus in a Feline Immunodeficiency Virus (FIV) packaging system and in GP2-293 (Clontech), a commercially available Moloney murine leukemia virus (MoMLV) packaging cell line system. A significant drawback to these packaging systems is that they do not produce a large number of particles comprising a glycoprotein from a BSL-4 virus. As a result, current packaging systems are impractical for the development and manufacture of vaccines as well as for high-throughput laboratory research.

In addition to being inefficient, current pseudovirus packaging systems can also be cumbersome to use. For instance, the widely used FIV packaging system requires co-transfection of three vectors containing 1) a FIV replicon (to be packaged), 2) gag and pol genes and 3) viral glycoprotein of interest.

The inventors of the present invention have overcome these barriers by developing novel MoMLV packaging cell lines that are easy to use and generate high titers of pseudotyped MoMLV viral particles. The new packaging cell lines allow for the packaging of target viral glycoprotein into pseudotyped viral particles, including pseudotyped replicon-deficient viral particles, and chimeric particles containing heterologous viral nucleoproteins. These cell lines can be used to express a variety of enveloped viruses, including, but not limited to, filoviruses, arenaviruses and bunyviruses.

### SUMMARY OF THE INVENTION

The present invention provides for a cell line comprising a stably integrated MoMLV gag gene, a stably integrated MoMLV pol gene and at least one heterologous viral glycoprotein gene. The cell line may further comprise an α(1,3) galactosyltransferase gene, for instance, a stably integrated mouse α(1,3) galactosyltransferase gene. The MoMLV gag gene, MoMLV pol gene and the α(1,3) galactosyltransferase gene may be constitutively expressed in the cell line, i.e., packaging cells. The at least one heterologous viral glycoprotein gene can be stably integrated or transiently expressed. Accordingly, the invention includes a cell line comprising a stably integrated MoMLV gag gene, a stably integrated MoMLV pol gene, a stably integrated mouse α(1,3) galactosyltransferase gene and at least one transiently expressed heterologous viral glycoprotein gene. In some embodiments, the cell line further comprises a nucleoprotein gene from an enveloped virus.

In one embodiment of the invention, the cell line lacks a viral replicon and produces replicon-deficient viral particles. For instance, the invention includes a cell line comprising a stably integrated MoMLV gag gene, a stably integrated MoMLV pol gene and at least one heterologous viral glycoprotein gene, wherein the cell line does not contain a viral replicon. The invention also includes a cell line comprising a stably integrated MoMLV gag gene, a stably integrated MoMLV pol gene, a stably integrated α(1,3) galactosyltransferase gene and at least one heterologous viral glycoprotein gene, wherein the cell line does not contain a viral replicon. The cell line may further comprise a nucleoprotein gene from a heterologous virus.

In yet another embodiment of the invention, the cell line comprising a stably integrated MoMLV gag gene, a stably integrated MoMLV pol gene and at least one heterologous viral glycoprotein gene further comprises a MoMLV viral replication-defective genome containing a reporter gene. In another embodiment, the cell line may further comprise a α(1,3) galactosyltransferase gene. The MoMLV gag gene, MoMLV pol gene, MoMLV viral replication-defective genome and the α(1,3) galactosyltransferase gene may be constitutively expressed in the cell line, i.e., packaging cells. The heterologous viral glycoprotein gene can be stably integrated or transiently expressed. For example, the invention includes a cell line comprising a stably integrated MoMLV gag gene, a stably integrated MoMLV pol gene, a stably integrated MoMLV viral replication-defective genome with an Enhanced Green Fluorescent Protein reporter gene and at least one transiently expressed heterologous viral glycoprotein gene. The present invention also includes a cell line comprising a stably integrated MoMLV gag gene, a stably integrated MoMLV pol gene, a stably integrated α(1,3) galactosyltransferase gene, a stably integrated MoMLV viral replication-defective genome with an Enhanced Green Fluorescent Protein reporter gene and at least one heterologous viral glycoprotein gene.

The present invention also provides for a cell line that lacks both a pol gene and a viral replicon. In this embodiment of the invention, the cell line comprises a stably integrated MoMLV gag gene and at least one heterologous glycoprotein gene from an enveloped virus. The at least one heterologous glycoprotein gene can be stably integrated or transiently expressed. The cell line may further comprise an α(1,3) galactosyltransferase gene, for instance, a stably integrated mouse α(1,3) galactosyltransferase gene. Accordingly, the invention includes cell line comprising a stably integrated MoMLV gag gene, a stably integrated α(1,3) galactosyltransferase gene and at least one transiently expressed heterologous glycoprotein gene from an enveloped virus, wherein the cell line does not contain a pol gene and viral replicon. In one embodiment of the invention, the cell line comprises a stably integrated MoMLV gag gene, a stably integrated α(1,3) galactosyltransferase gene and at least one heterologous glycoprotein gene from an enveloped virus (e.g. high risk pathogen) selected from the group consisting of Ebola virus, Marburg virus, Lassa virus, Rift Valley fever virus and Crimean Congo hemorrhagic fever virus, wherein the cell line does not contain a pol gene and viral replicon, wherein the at least one heterologous glycoprotein gene is stably integrated or transiently expressed. In another embodiment, the cell line further comprises a nucleoprotein gene from a heterologous virus. The nucleoprotein gene may be stably integrated or transiently expressed.

The at least one heterologous viral glycoprotein gene of the present invention can be any enveloped viral glycoprotein gene or genes. In some embodiments, the heterologous viral glycoprotein gene may be a chimeric glycoprotein gene encoding a chimeric glycoprotein in which one or more domains of the native glycoprotein is replaced with one or more domains from a heterologous glycoprotein. In other embodiments, the heterologous viral glycoprotein gene may be codon-optimized for expression in mammalian cells. In one embodiment, the invention provides an isolated nucleotide sequence encoding a Rift Valley Fever virus glycoprotein, wherein said isolated polynucleotide sequence is codon-optimized for expression in a mammalian cell. In another embodiment, the invention provides an isolated nucleotide sequence encoding a Lassa virus glycoprotein, wherein said isolated polynucleotide sequence is codon-optimized for expression in a mammalian cell. In another embodiment of the invention, the at least one heterologous viral glycoprotein gene is part of a progene. In some embodiments, a progene may produce a precursor polypeptide that is post-translationally processed into two or more heterologous viral glycoprotein genes from the same enveloped virus. In another embodiment of the invention, two or more viral glycoprotein genes from two or more enveloped viruses are present in the cell line.

In one embodiment of the invention, the heterologous viral glycoprotein gene(s) are from a virus classified as a high risk pathogen (e.g. BSL-3 and/or BSL-4 viruses). In another embodiment, the heterologous viral glycoprotein gene(s) are from a BSL-4 virus (e.g. high risk pathogen) selected from the group consisting of bunyavirus (e.g., Hanta virus, Crimean Congo hemorrhagic fever virus and Rift Valley fever virus), filovirus (e.g., Ebola virus and Marburg virus) and arenavirus (e.g., Lassa virus).

In another embodiment, said heterologous glycoprotein gene is from an enveloped virus (e.g. high risk pathogen). In another embodiment, said enveloped virus is an arenavirus. In another embodiment, said arenavirus is a Lassa virus. In another embodiment, said enveloped virus is a filovirus. In another embodiment, said filovirus is an Ebola virus. In another embodiment, said filovirus is a Marburg virus. In yet another embodiment, said enveloped virus is a bunyavirus. In one embodiment, said bunyavirus is Rift Valley fever virus. In another embodiment, said bunyavirus is Crimean Congo hemorrhagic fever virus.

In one embodiment of the invention, the at least one heterologous viral glycoprotein gene is transiently expressed in the cell line. The cell line can be transiently transfected with one or more plasmids encoding one or more heterologous viral glycoprotein genes of interest. In another embodiment of the invention, the at least one heterologous viral glycoprotein gene is stably integrated into the MoMLV genome. The heterologous viral glycoprotein gene or genes of the present invention may be expressed from an inducible promoter.

The cell line of the invention is capable of producing a large titer of pseudovirions. In one embodiment, said cell line generates a titer of at least about 1.0×10⁵ cfu/ml, at least about 5.0×10⁵ cfu/ml, at least about 7.0×10⁵ cfu/ml, at least about 9.0×10⁵ cfu/ml, at least about 1.0×10⁶ cfu/ml, at least about 1.0×10⁷ cfu/ml, or at least about 1.0×10⁸ cfu/ml pseudovirions. In one embodiment of the invention, the pseudovirions are replicon-deficient viral particles (i.e., particles generated from the cell lines that do not contain a viral replicon). The invention includes a cell line which generates a titer of at least about 1.0×10⁵ cfu/ml, at least about 5.0×10⁵ cfu/ml, at least about 7.0×10⁵ cfu/ml, at least about 9.0×10⁵ cfu/ml, at least about 1.0×10⁶ cfu/ml, at least about 1.0×10⁷ cfu/ml, or at least about 1.0×10⁸ cfu/ml replicon-deficient viral particles.

The present invention provides for a cell line comprising a stably integrated MoMLV gag, a stably integrated MoMLV pol, a stably integrated MoMLV replication-defective genome, a stably integrated mouse α(1,3) galactosyltransferase gene and at least one transiently expressed heterologous viral glycoprotein gene. In one embodiment, said at least one heterologous glycoprotein gene encodes for a Lassa virus glycoprotein. In another embodiment, said at least one heterologous glycoprotein gene encodes for an Ebola virus glycoprotein. In another embodiment, said at least one heterologous glycoprotein gene encodes for a Marburg virus glycoprotein. In yet another embodiment, said at least one heterologous glycoprotein gene codes for a Crimean Congo hemorrhagic fever virus glycoprotein or a Rift Valley fever virus glycoprotein.

The present invention also provides for an antigenic preparation for inducing an immune response against a high risk pathogen (e.g. enveloped virus), wherein said antigenic preparation comprises replicon-deficient viral particles produced by the cell lines of the invention. In one embodiment, the antigenic preparation comprises a MoMLV protease, reverse transcriptase, integrase, capsid and nucleocapsid proteins and at least one heterologous surface glycoprotein. In another embodiment, the antigenic preparation comprises MoMLV gag proteins and at least one heterologous surface glycoprotein. In some embodiments, the antigenic preparation may further comprise a heterologous nucleoprotein. In one embodiment, said at least one heterologous glycoprotein is a Lassa virus glycoprotein. In another embodiment, said at least one heterologous glycoprotein is an Ebola virus glycoprotein or Marburg virus glycoprotein. In one embodiment, the at least one heterologous glycoprotein is a Rift Valley fever virus glycoprotein. In another embodiment, the at least one heterologous glycoprotein is a Crimean Congo hemorrhagic fever virus glycoprotein. In another embodiment, said at least one heterologous glycoprotein comprises αGal epitopes. In another embodiment, the at least one heterologous glycoprotein is a chimeric glycoprotein.

The present invention also provides for a vaccine preparation for inducing an immune response against a high risk pathogen (e.g. enveloped virus), wherein said vaccine preparation comprises replicon-deficient viral particles produced by the cell lines of the invention. In one embodiment, the vaccine preparation comprises a MoMLV protease, reverse transcriptase, integrase, capsid and nucleocapsid proteins and at least one heterologous surface glycoprotein. In another embodiment, the vaccine preparation comprises MoMLV gag proteins and at least one heterologous surface glycoprotein. In some embodiments, the vaccine preparation may further comprise a heterologous nucleoprotein. In one embodiment, said at least one heterologous glycoprotein is a Lassa virus glycoprotein. In another embodiment, said at least one heterologous glycoprotein is an Ebola virus glycoprotein. In another embodiment, the at least one heterologous glycoprotein is a Rift Valley fever virus glycoprotein. In another embodiment, the at least one heterologous glycoprotein is a chimeric glycoprotein. In another embodiment, the at least one heterologous glycoprotein comprises αGal epitopes. In some embodiments, the vaccine preparation further comprises an adjuvant.

The present invention also provides for a method of preparing vaccines for protection against a high risk pathogen infection comprising the steps of transfecting at least one heterologous viral glycoprotein gene into a cell line comprising a stably integrated MoMLV gag, collecting and concentrating the pseudovirions; and resuspending said pseudovirions in a pharmaceutically acceptable buffer. In one embodiment, the cell line further comprises a stably integrated MoMLV pol gene. In another embodiment, the cell line further comprises a stably integrated MoMLV pol gene and a stably integrated viral replication-defective genome. In another embodiment, the cell line further comprises a heterologous viral nucleoprotein gene. The at least one heterologous glycoprotein gene may encode a Lassa virus glycoprotein. In another embodiment, said at least one heterologous glycoprotein gene encodes an Ebola virus glycoprotein. In another embodiment, said at least one heterologous glycoprotein gene encodes a Rift Valley fever virus glycoprotein or a Crimean Congo hemorrhagic fever virus. In another embodiment, the at least one heterologous glycoprotein gene is a chimeric glycoprotein gene. In another embodiment, αGal epitopes are either chemically or enzymatically added to said pseudovirions.

### BRIEF DESCRIPTION OF THE DRAWINGS

FIG. 1 depicts the map of pGP-IRES-Zeo which is being used to integrate the gag and pol genes into 293 cells for the production of a MoMLV packaging cell line. A vector containing gag and pol was used to generate the packaging cells.

FIG. 2 depicts the map of MoMLV replication-defective genome pLEIN (pLEGFP-IRES-Neo). This vector was used to stably integrate the 5′ and 3′ LTRs and packaging signal into the cells to generate a MoMLV packaging cell line (e.g., 1F5) which allows pseudovirion production upon transfection of an expression plasmid encoding the target glycoprotein.

FIG. 3 depicts GFP Fluorescence Analysis (100× magnification) of A: Vero cells only, B: medium from untransfected 1F5 cells on Vero Cells, C: non-transfected 1F5 cell, D: medium from EBOV-GP transfected 1F5 on Vero cells, E: medium from MARV-GP transfected 1F5 on Vero cells, F: medium from MARV-GP transfected 1F5 on Vero cells.

FIG. 4 depicts generation of pseudotyped MoMLV using a newly established packaging system. A: Comparison of pseudovirion titers generated in commercially available GP 293 cells (white) or producer clone 1F5 grey. The producer cell line 1F5 was transiently transfected with VSV-GP LV-GP only, whereas the MoMLV GFP replication-defective genome and these glycoprotein expression vectors were both transfected into GP 293 cells. Subsequently, pseudovirions were collected, filtered, applied to 293 cells, and titered in triplicate using FACS. MoMLV pseudotyped with LV GP titers improved by two logs using the selected MoMLV producer clone 1F5. B: The 1F5 MoMLV packaging system was used to generate pseudotype virions containing EBOV, LV, MARV or VSV glycoproteins. An additional Kozak sequence immediately upstream of the start codon in the LV-GP expression plasmid (Koz-LV) increased the pseudovirus titer by approximately one log. C: Time kinetic experiment to determine the optimal pseudovirus harvest time point post-transfection of the MoMLV packaging cell line 1F5.

FIG. 5 depicts generation of very high titer EBOV-MoMLV via concentration of pseudovirion particles. Pseudovirions harvested from transfected 1F5 cells were concentrated 100× via centrifugation at 77,000×g through a 20% sucrose cushion (2×). Titer was determined via FACS analysis of Vero cells transduced with 10.0 μl (1×) and 10-fold dilutions of concentrated particles, as well as 1.0 ml of unconcentrated harvested pseudovirions. Titers reached>1×10⁸ particles/mL.

FIG. 6 depicts generation of LV-pseudotyped MoMLV using a newly established packaging system BPSC-1. The newly established BPSC-1 packaging system was used to generate pseudotype virions containing LV glycoproteins. As a positive control, the 2E6 MoMLV packaging system (sister clone of 1F5) was also used to generate pseudotype virions containing LV glycoproteins.

FIG. 7. αGal trisaccharide and α(1,3)GT stably expressing cells. A: Schematic presentation of the Galactose-alpha(1,3)-galactose-beta(1,4)N-acetylglucosamine-R [Gal-α(1-3)-Gal-β(1,4)-GlcNAc-R] (αGal) epitope. B: FACS analysis of 293 cells (upper panel) and 293αGal+ cells constitutively expressing the α1,3 Galactosyl Transferase gene (lower panel). Cells were stained with chicken anti-αGal antibodies (NewLink Genetics Corporation), a secondary biotinylated rabbit anti-chicken IgY antibody followed by Streptavidin PE. No signal is detected on 293 cells while greater than 90% of the 293αGal+ cells are strongly positive for αGal epitopes.

FIG. 8. Enzymatic modification with αGal epitopes. The activity of recombinant α1,3 GT activity was confirmed by in vitro modification of fetuin. ELISA plates coated with 20 μg/ml fetuin (Sigma F2379) were incubated with the following reagents: Lanes A, B and C contained recombinant α1,3 GT; lanes A, B and D contained UDP-Gal; Lane B was treated with neuraminidase; and Lane D contained PBS as a negative control. Five different α1,3 GT enzyme preparations were tested (rows 1 to 5): 1: Native protein, 2:1 mM DTT added to binding buffer, 3:10 mM DTT added to binding buffer, 4: Denatured α1,3 GT, and 5: Denatured and refolded α1,3 GT. A representative ELISA plate is shown on the left. The absorbance value at 450 nm for each well is shown on the right.

FIG. 9. Comparison of different modification techniques for the addition of αGal epitopes to viral antigens. Each well of a microtiter plate was coated with either 0.25 μg of Chiron's Influrin Influenza hemagglutinins (HA) peptides (H1N1, H3N2, Influenza B) or albumin as follows: A: αGal-conjugated human serum albumin; B: human serum albumin; C: Influenza HAs from Chiron vaccine Influvirin chemically modified with αGal epitopes (Dextra Laboratories Limited); D: HAs modified by α1,3 GT enzyme (Sigma-Aldrich); E: HAs modified by NewLink α1,3 GT(1); F: HAs modified by NewLink α1,3 GT(2); G: HAs treated as in D, E, F but without α1,3 GT enzyme; and H: untreated HAs. αGal modification was detected by serial dilutions of biotin-conjugated lectin from 1/200× (125 ng/50 μl) to 1/3,200× (columns 1-10). Columns 11 and 12 did not contain primary antibody. Streptavidin-conjugated horse radish peroxidase was used to bind biotin-conjugated lectin followed by reaction with substrate. A representative ELISA plate is shown on the left. The absorbance value at 450 nm for each well is shown on the right.

FIG. 10. Detection of αGal modification in antiviral vaccines by anti-αGal ELISA approach. A: Analysis of Lassa virus glycoprotein (GP)-Moloney murine leukemia virus (MoMLV) generated in αGal+ packaging cells. B: Analysis of two different Ebola Zaire GP-MoMLV preparations generated in αGal+ packaging cells. Chicken anti-αGal antibodies were used to probe microplate wells coated with solubilized virion proteins. Medium and rabbit red blood cells (RRBCs) were used as negative and positive control coating agents, respectively.

FIG. 11. Quantification of αGal content on virus stocks grown in αGal+ cells. A: Results of an anti-αGal ELISA using chicken anti-αGal Abs with Rift Valley fever virus MP12 strain propagated in αGal+(stably expressing α1,3 GT) or αGal− Vero cells. B: Linear regression for dilution of αGal+MP12 to generate a standard curve for subsequent quantification of αGal content.

FIG. 12. Normal human serum neutralization assay to demonstrate αGal modification of vaccine candidates. Normal (panels A and C) or heat-inactivated (panels B and D) human serum was added to infectious pseudovirus preparations, and inactivation was measured by a reduction in the virus-mediated transfer of a GFP reporter gene (located in a packaged replication-defective genome) to target cells.

FIG. 13. Characterization of established MoMLV packaging cell line. Different transfection strategies (panel A) and amounts of transfected EBOV glycoprotein expression plasmid (panels B and C) were tested to characterize and optimize pseudovirus production.

FIG. 14. Analysis of pseudovirus integrity using an anti-Gag antisera. Different pseudovirion preparations were used to analyze MoMLV gag content. Blot was probed with anti-Gag 1:5000.

FIG. 15. Western Blot of two separate purified RVFV G-MoMLV preparations probed with an anti-G_{N} antisera. Blot was blocked in 2% non-fat dry milk in PBS overnight followed by exposure to rabbit anti-RVFV G_{N}. The blot was then incubated with alkaline phosphatase-conjugated donkey anti-rabbit antibodies and visualized with a one-step developer (Pierce). The band at approximately 56 kD corresponds to RVFV G_{N} present in the MoMLV pseudovirions.

FIG. 16. Immunofluorescent localization of wildtype and chimeric RVFV G_{N}. The MoMLV packaging cell line was transfected with wild type (WT) G_{N} (right panel) or a chimeric RVFV G_{N} in which the cytoplasmic domain was exchanged with the cytoplasmic domain (TR) from MoMLV envelope protein (left panel). Cells were fixed and probed with an anti-RVFV G_{N} polyclonal sera to detect localization of the RVFV G_{N} protein to the plasma membrane.

FIG. 17. Efficacy of RVFV GP-MoMLV in α1,3 GT-KO Mouse Model. α1,3 GT-KO mice were immunized s. c. with αGal-modified or unmodified RVFV GP-pseudotyped MoMLV, followed by boosters at 2-week intervals. Mice were then challenged 7 weeks post first vaccination with 100 pfu RVFV ZH501. Control mice received no vaccine or EBOV-GP-pseudotyped MoMLV. A: Effect of αGal-modified and unmodified vaccine with adjuvant on survival after challenge with lethal dose of RVFV. B: Effect of 1/10 dose reduction of αGal-modified vaccine on survival after challenge with lethal dose of RVFV. C: Effect of reduction in number of vaccine administrations on survival after challenge with lethal dose of RVFV. D: Efficacy of RVFV GP-pseudotyped MoMLV and RVF virus-like particles (VLPs containing MoMLV gag and RVFV G with or without the RVFV nucleoprotein) in a live challenge mouse model. VLPs were generated in α1,3 GT+293 cells.

FIG. 18. Co-localization of viral glycoproteins and αGal epitopes on the surface of pseudovirions. αGal+EBOV GP-pseudotyped MoMLV (upper panels) and LV GP-pseudotyped MoMLV (lower panels) were probed with rabbit anti-EBOV GP (left panels) and chicken anti-αGal antibodies (right panels). The primary antibodies were subsequently visualized with anti-rabbit and anti-chicken antibodies coupled to small and large gold particles, respectively.

FIG. 19. Efficacy testing of αGal-modified pseudovirions. A: α1,3 GT-KO mice were immunized s.c. with 10⁶ (1 and 2) or 10⁷ (3 and 4) pfu of αGal-modified (2 and 4) or unmodified (1 and 3) EBOV GP-pseudotyped MoMLV. Control mice received PBS (5). Secreted cytokines were measured in culture supernatants from PBMCs isolated from vaccinated mice six days post-injection. Error bars indicate standard deviations (n=2). B: α1,3 GT-KO mice were immunized s.c. with 10⁷αGal-modified (2) or unmodified (1) LV GP-pseudotyped MoMLV, and controls received PBS (3). PBMCs were isolated from five mice, processed, cultured as in A but in the presence of unmodified LV GP-pseudotyped MoMLV. Culture supernatants were analyzed as in A. Error bars indicate standard deviations (n=2).

FIG. 20. Efficacy of EBOV GP-MoMLV in α1,3 GT-KO Mouse Model. α1,3 GT-KO mice were immunized s.c. with 10⁷ (1E7) or 10⁵ (1E5) unmodified EBOV GP-pseudotyped MoMLV, followed by 2 boosters at 2-week intervals. Mice were then challenged 3 weeks post final vaccination with 100 pfu mouse-adapted Zaire Ebolavirus (MA ZEBOV). Control mice received no vaccine.

FIG. 21. αGal-modified and unmodified EBOV GP-MoMLV in α1,3 GT-KO Mouse Model. α1,3 GT-KO mice were immunized s.c. with 10⁷ pfu αGal-modified or unmodified EBOV GP-pseudotyped MoMLV, followed by boosters at 2-week intervals (one or two). Mice were then challenged 7 weeks post first vaccination with 100 pfu mouse-adapted Ebolavirus (MA EBOV). Control mice received no vaccine.

FIG. 22. Expression of codon-optimized RVFV glycoprotein in mammalian cells and generation of codon-optimized RVFV GP-containing MoMLV pseudoviruses. A:Western blot analysis of lysates of 293 cells transfected with the indicated quantities of expression plasmids containing either native or codon-optimized RVFV glycoprotein. The blot was probed with an anti-RVFV G_{N} monoclonal Ab and visualized with AP-conjugated goat-anti-mouse antibodies. B: Western blot analysis of purified pseudovirus obtained from MoMLV producer cells transfected with ATG CO (codon-optimized RVFV glycoprotein; 1) or ATG4 (native RVFV glycoprotein; 2). The blot was probed with an anti-RVFV G_{N} monoclonal Ab and visualized with AP-conjugated goat-anti-mouse antibodies.

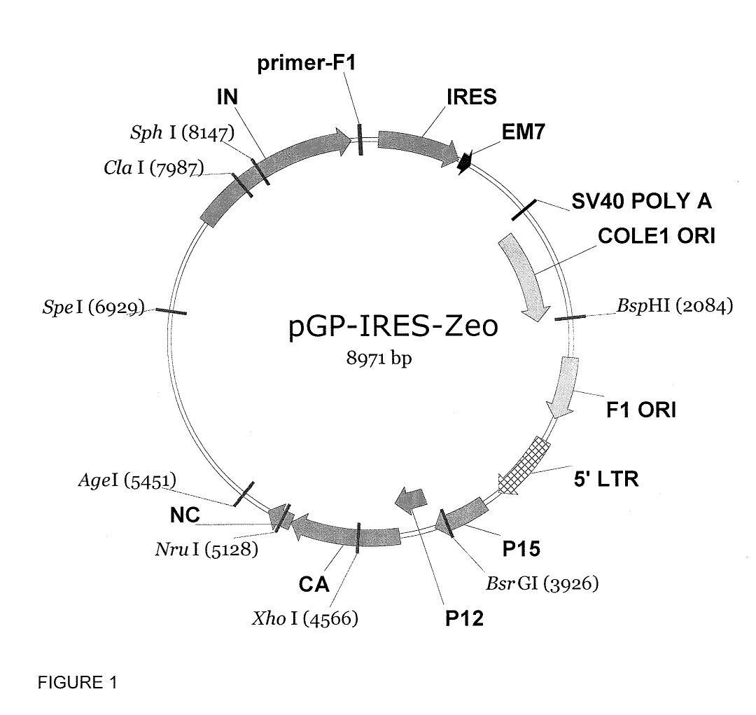

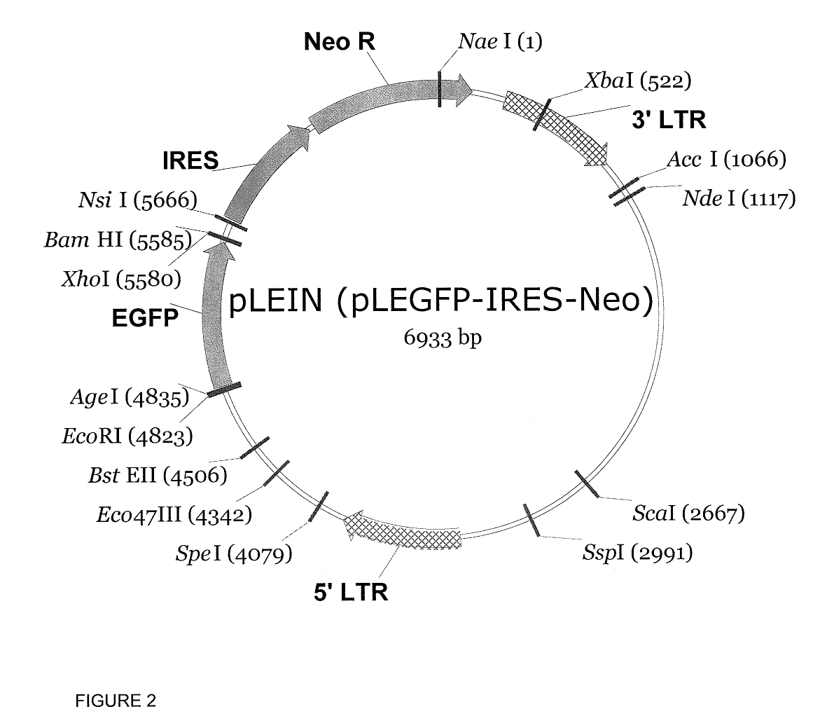

### DETAILED DESCRIPTION

### Virus Particles Packaging Cell Lines and Virus Particles of the Invention

The invention comprises a novel cell-based virus particle packaging system for the generation of replication-incompetent MoMLV virions packaged with at least one heterologous viral glycoprotein, e.g. filovirus, arenavirus and/or bunyavirus glycoproteins. This system allows for the generation of high-titer pseudotyped MoMLV virions (MoMLV pseudovirion) expressing at least one surface glycoprotein of a heterologous virus of interest. As used herein, “high-titer” means a titer of at least about 1.0×10⁵ cfu/ml, at least about 5.0×10⁵ cfu/ml, at least about 7.0×10⁵ cfu/ml, at least about 9.0×10⁵ cfu/ml, at least about 1.0×10⁶ cfu/ml or more. Having a cell-based virus particle packaging system that makes high titer pseudovirions is important for mass distribution of these particles (e.g., for vaccine production). These particles must be produced at a sufficient quantity and at a commercially viable cost. Even incremental increases in productivity can be economically significant. Thus, the cell-based virus particle packaging system of the invention will cut the cost of manufacturing such particles.

As used herein, pseudovirion refers to a viral particle containing one or more viral glycoproteins of interest. Pseudovirion, pseudovirus and virus-like particle are used interchangeably herein and refer to particles that comprise one or more structural proteins (e.g. nucleocapsid and capsid proteins) and at least one viral glycoprotein. A “replication-defective genome” or “replicon” refers to a viral nucleic acid lacking one or more functional genes required for generation of progeny virus. A replication-defective genome or replicon may contain a reporter gene in place of one or more viral genes. A “replicon-deficient viral particle”, as used herein, is a pseudovirion or virus-like particle that lacks a replicon. A replicon-deficient viral particle can also be an empty particle, i.e. does not contain any nucleic acid.

The ability to generate pseudotyped MoMLV virions such as replicon-deficient viral particles is also important and advantageous when studying viruses that are categorized as highcontainment, e.g. BSL 3 and 4 pathogens, including bunyaviruses (e.g., Rift Valley fever virus and Crimean Congo hemorrhagic fever virus), filoviruses (e.g., Ebola virus and Marburg virus) and arenaviruses (e.g., Lassa virus, Sabia virus, Machupo virus, Junin virus, and Guanarito virus), because MoMLV pseudovirions are non-pathogenic and can be handled at BSL 2. Importantly, because the immune system will respond to the glycoproteins from the BSL 4 pathogens expressed on the pseudovirions of the invention, they represent an ideal platform for the development of pseudovirion-based anti-viral vaccines for BSL 4 pathogens.

A “high risk pathogen”, as used herein, is a pathogen (e.g. virus) that requires high containment facilities (e.g. BSL 3 or BSL 4) and special safety precautions as defined in the Biosafety in Microbiological and Biomedical Laboratories manual published by the Centers for Disease Control (CDC). A high risk pathogen may also refer to a pathogen, such as a virus, that is classified as a high containment pathogen or a Category A, B or C High Risk Pathogen as defined by the CDC and NIH/NAIAD. Generally, high risk pathogens are pathogens that pose a high individual risk of life-threatening disease, which may be transmitted via the aerosol route and for which there is no vaccine or therapy. Some examples of high risk pathogens include, but are not limited to, members of the Filoviridae (Ebola and Marburg), Arenaviridae (Lassa, Sambia, Guanarito, Junin, and Machupo), Bunyaviridae (Rift Valley Fever, Crimean Congo Hemorrhagic Fever), and Paramyxoviridae (Nipah and Hendra) families of viruses. In addition, viruses that have similar or identical antigenic relationships to the viruses listed above are also initially classified high risk pathogens. Viruses that fall into Risk Category 4 (described in the Biosafety in Microbiological and Biomedical Laboratories manual, Centers for Disease Control) that are transmissible through aerosols or have unknown routes of transmission can also be classified as high risk pathogens. The categorization of a high risk pathogen may be time-dependent, as many viruses initially categorized as high risk pathogens are later reclassified after a vaccine is developed, or after more is known about the virus.

Thus, the invention includes a cell line comprising, a stably integrated MoMLV gag, a stably integrated MoMLV pol, optionally a stably integrated MoMLV replication-defective genome and at least one gene encoding a viral glycoprotein. As used herein, stably integrated refers to the integration of a nucleic acid (also referred to interchangeably as gene and polynucleic acid herein) into the host cell's genome such that the nucleic acid persists as part of the cellular genome even after several cell divisions. In one embodiment of the invention, the cell line produces replicon-deficient viral particles. In another embodiment, the cell line does not contain a MoMLV replicon.

In another embodiment, the cell line does not contain a viral pol gene. In another embodiment, the cell line does not contain a MoMLV pol gene. In yet another embodiment, the cell line does not contain a pol gene or replicon.

In one embodiment, said gene encoding the heterologous glycoprotein is transiently expressed in the host cell. In another embodiment, said heterologous glycoprotein is stably integrated into the cell genome. In some embodiments, the heterologous glycoprotein is expressed from an inducible promoter to decrease toxicity to the cell. Inducible promoters are known in the art, and include, but are not limited to tetracycline promoter, metallothionein IIA promoter, heat shock promoter, steroid/thyroid hormone/retinoic acid response elements, the adenovirus late promoter, and the inducible mouse mammary tumor virus LTR.

Non-limiting examples of viruses from which said heterologous glycoprotein can be derived from are the following: Seasonal, Avian or Pandemic Influenza (A and B, e.g. HA and/or NA); Coronavirus (e.g., SARS); Hepatitis viruses A, B, C, D and E3; Human Immunodeficiency Virus (HIV); Herpes viruses 1, 2, 6 and 7; Cytomegalovirus; Varicella Zoster; Papilloma virus; Epstein Barr virus; Adenoviruses; Bunya viruses, including, but not limited to, Crimean Congo hemorrhagic fever virus, Rift Valley fever virus or La Crosse virus, Hanta virus or any other emerging bunyavirus; Coxsakie viruses; Picoma viruses; Rotaviruses; Rhinoviruses; Rubella virus; Polio virus (multiple types); Adeno virus (multiple types); Parainfluenza virus (multiple types); Shipping fever virus; Western and Eastern Equine Encephalomyelitis; Japanese Encephalomyelitis; Fowl Pox; Rabies Virus; Slow Brain viruses; Rous Sarcoma virus; Papovaviridae; Parvoviridae; Picornaviridae; Poxyiridae (such as Smallpox or Vaccinia); Reoviridae (e.g., Rotavirus); Retroviridae (e.g., HTLV-I, HTLV-II and Lentivirus); Togaviridae (e.g., Rubivirus); Respiratory Syncytial virus (RSV): West Nile fever virus; Flavivirus, including, but not limited to, Dengue virus (all serotypes); Russian Spring Summer virus; Yellow fever virus; Kyasanur Forest Disease virus; Omsk hemorrhagic fever virus; West Nile virus; Tick-borne Encephalitis virus; Japanese Encephalitis virus; Chikungunya virus or similar viruses or any other emerging flavivirus; filovirus, including, but not limited to, Ebola virus (EBOV) or Marburg virus (MARV) or any other emerging filovirus; arenavirus, including, but not limited to, Lassa virus (LV) or Lymphocytic Choriomeningitis virus (LCMV) or one of the New World arenaviruses including, but not limited to, Sabia, Guanarito, Junin or Machupo or any other emerging arenavirus; Paramyxovirus including, but not limited to, Nipah virus, Hendra virus or any other emerging Henipavirus; Measles; mumps; and Rhabdoviridae, including, but not limited to Rabies and VSV.

In another embodiment, the invention includes a cell line comprising a stably integrated MoMLV gag, a stably integrated MoMLV pol, a stably integrated MoMLV replication-defective genome and a gene encoding an Arena virus glycoprotein, including, but not limited to a Lassa glycoprotein. In another embodiment, said Arena virus glycoprotein gene (such as Lassa virus glycoprotein gene) is transiently expressed in the host cell. In another embodiment, said Arena virus glycoprotein gene (such as Lassa virus glycoprotein gene) is stably integrated into the cell genome. In another embodiment, the invention includes a cell line consisting essentially of a stably integrated MoMLV gag gene, a stably integrated MoMLV pol gene, a stably integrated MoMLV replication-defective genome and a gene encoding an Arena virus glycoprotein (such as a Lassa virus glycoprotein). In another embodiment, the invention includes a cell line consisting of a stably integrated MoMLV gag gene, a stably integrated MoMLV pol gene, a stably integrated MoMLV replication-defective genome and a gene encoding an Arena virus glycoprotein (such as a Lassa virus glycoprotein). The gene encoding a Lassa virus glycoprotein may be a progene. A “progene”, as used herein, is a viral gene that encodes a protein precursor, which is post-translationally processed to yield two or more proteins. The Lassa virus glycoprotein may be the GPC gene (i.e. progene), which encodes glycoprotein 1 (GP1) and glycoprotein 2 (GP2).

In another embodiment, the invention includes a cell line comprising a stably integrated MoMLV gag, a stably integrated MoMLV pol, a stably integrated MoMLV replication-defective genome and a gene encoding a filovirus glycoprotein, including, but not limited to an Ebola virus glycoprotein or Marburg virus glycoprotein. In another embodiment, said filovirus glycoprotein gene (such as an Ebola virus glycoprotein gene or Marburg virus glycoprotein gene) is transiently expressed in the host cell. In another embodiment, said filovirus glycoprotein gene (such as an Ebola virus glycoprotein gene or Marburg virus glycoprotein gene) is stably integrated into the cell genome. In another embodiment, the invention includes a cell line consists essentially of a stably integrated MoMLV gag, a stably integrated MoMLV pol, a stably integrated MoMLV replication-defective genome and a filovirus glycoprotein gene (such as an Ebola virus glycoprotein gene or Marburg virus glycoprotein gene). In another embodiment, the invention includes a cell line consisting of a stably integrated MoMLV gag, a stably integrated MoMLV pol, a stably integrated MoMLV replication-defective genome and a filovirus glycoprotein gene (such as an Ebola virus glycoprotein gene or Marburg virus glycoprotein gene). The gene encoding a filovirus glycoprotein may be a progene, wherein the progene generates a protein precursor which is post-translationally processed into two or more filovirus glycoproteins, e.g. GP1 and GP2.

In one embodiment, the invention includes a cell line comprising a stably integrated MoMLV gag, a stably integrated MoMLV pol, a stably integrated MoMLV replication-defective genome and a gene encoding a bunyavirus glycoprotein, including, but not limited to a Rift Valley fever virus glycoprotein or Crimean Congo hemorrhagic fever virus glycoprotein. In another embodiment, said bunyavirus glycoprotein gene (such as Rift Valley fever virus glycoprotein gene or Crimean Congo hemorrhagic fever virus glycoprotein gene) is transiently expressed in the host cell. In another embodiment, said bunyavirus glycoprotein gene (such as Rift Valley fever virus glycoprotein gene or Crimean Congo hemorrhagic fever virus glycoprotein gene) is stably integrated into the cell genome. In another embodiment, the invention includes a cell line consisting essentially of a stably integrated MoMLV gag, a stably integrated MoMLV pol, a stably integrated MoMLV replication-defective genome and a gene encoding a bunyavirus glycoprotein (such as Rift Valley fever virus glycoprotein gene or Crimean Congo hemorrhagic fever virus glycoprotein gene). In another embodiment, the invention includes a cell line consisting of a stably integrated MoMLV gag, a stably integrated MoMLV pol, a stably integrated MoMLV replication-defective genome and a gene encoding a bunyavirus glycoprotein (such as Rift Valley fever virus glycoprotein gene or Crimean Congo hemorrhagic fever virus glycoprotein gene). The gene encoding a bunyavirus glycoprotein may be a progene, wherein the progene generates a protein precursor, which is post-translationally processed into two or more bunyavirus glycoproteins. In one embodiment, the gene encoding a Rift Valley fever virus glycoprotein is a progene, wherein said progene produces a precursor, which is post-translationally processed to yield Rift Valley fever virus glycoproteins, e.g. G_{N} and G_{C}.

The invention includes cell lines which produce replicon-deficient viral particles. For instance, the invention includes a cell line comprising a stably integrated MoMLV gag, a stably integrated MoMLV pol, and a gene encoding a bunyavirus glycoprotein, a filovirus glycoprotein and/or an arenavirus glycoprotein.

In another embodiment, the invention includes a cell line which lacks a replicon and which comprises a stably integrated MoMLV gag, a stably integrated MoMLV pol and a gene encoding an arenavirus glycoprotein, including, but not limited to a Lassa glycoprotein.

In another embodiment, said arenavirus glycoprotein gene (such as Lassa virus glycoprotein gene) is transiently expressed in the host cell. In another embodiment, said arenavirus glycoprotein gene (such as Lassa virus glycoprotein gene) is stably integrated into the cell genome. In another embodiment, the invention includes a cell line which lacks a replicon and which consists essentially of a stably integrated MoMLV gag gene, a stably integrated MoMLV pol gene and a gene encoding an arenavirus glycoprotein (such as a Lassa virus glycoprotein). In another embodiment, the invention includes a cell line which lacks a replicon and consists of a stably integrated MoMLV gag gene, a stably integrated MoMLV pol gene and a gene encoding an arenavirus glycoprotein (such as a Lassa virus glycoprotein). In another embodiment, the invention includes a cell line which lacks a replicon and consists of a stably integrated MoMLV gag gene, a stably integrated MoMLV pol gene, a stably integrated α(1,3) galactosyltransferase gene and a gene encoding an arenavirus glycoprotein (such as a Lassa virus glycoprotein).

In another embodiment, the invention includes a cell line which lacks a replicon and comprises a stably integrated MoMLV gag gene, a stably integrated MoMLV pol gene and a gene encoding a filovirus glycoprotein, including, but not limited to an Ebola virus glycoprotein or Marburg virus glycoprotein. In another embodiment, said filovirus glycoprotein gene (such as an Ebola virus glycoprotein gene or Marburg virus glycoprotein gene) is transiently expressed in the host cell. In another embodiment, said filovirus glycoprotein gene (such as an Ebola virus glycoprotein gene or Marburg virus glycoprotein gene) is stably integrated into the cell genome. In another embodiment, the invention includes a cell line which lacks a replicon and consists essentially of a stably integrated MoMLV gag gene, a stably integrated MoMLV pol gene and a filovirus glycoprotein gene (such as an Ebola virus glycoprotein gene or Marburg virus glycoprotein gene). In another embodiment, the invention includes a cell line which lacks a replicon and consists of a stably integrated MoMLV gag gene, a stably integrated MoMLV pol gene and a filovirus glycoprotein gene (such as an Ebola virus glycoprotein gene or Marburg virus glycoprotein gene). In another embodiment, the invention includes a cell line which lacks a replicon and consists of a stably integrated MoMLV gag gene, a stably integrated MoMLV pol gene, a stably integrated α(1,3) galactosyltransferase gene and a filovirus glycoprotein gene (such as an Ebola virus glycoprotein gene or Marburg virus glycoprotein gene).

In one embodiment, the invention includes a cell line which lacks a replicon and comprises a stably integrated MoMLV gag gene, a stably integrated MoMLV pol gene and a gene encoding a bunyavirus glycoprotein, including, but not limited to a Rift Valley fever virus glycoprotein or Crimean Congo hemorrhagic fever virus glycoprotein. In another embodiment, said bunyavirus glycoprotein gene (such as Rift Valley fever virus glycoprotein gene or Crimean Congo hemorrhagic fever virus glycoprotein gene) is transiently expressed in the host cell. In another embodiment, said bunyavirus glycoprotein gene (such as Rift Valley fever virus glycoprotein gene or Crimean Congo hemorrhagic fever virus glycoprotein gene) is stably integrated into the cell genome. In another embodiment, the invention includes a cell line which lacks a replicon and consists essentially of a stably integrated MoMLV gag gene, a stably integrated MoMLV pol gene and a gene encoding a bunyavirus glycoprotein (such as Rift Valley fever virus glycoprotein gene or Crimean Congo hemorrhagic fever virus glycoprotein gene). In another embodiment, the invention includes a cell line which lacks a replicon and which consists of a stably integrated MoMLV gag gene, a stably integrated MoMLV pol gene and a gene encoding a bunyavirus glycoprotein (such as Rift Valley fever virus glycoprotein gene or Crimean Congo hemorrhagic fever virus glycoprotein gene). In another embodiment, the invention includes a cell line which lacks a replicon and which consists of a stably integrated MoMLV gag gene, a stably integrated MoMLV pol gene, a stably integrated α(1,3) galactosyltransferase gene and a gene encoding a bunyavirus glycoprotein (such as Rift Valley fever virus glycoprotein gene or Crimean Congo hemorrhagic fever virus glycoprotein gene).

The invention includes cell lines which lack both a pol gene and a replicon and which produce replicon-deficient viral particles. For instance, the invention includes a cell line comprising a stably integrated MoMLV gag and a gene encoding a bunyavirus glycoprotein, a filovirus glycoprotein and/or an arenavirus glycoprotein.

In another embodiment, the invention includes a cell line which lacks a replicon and a MoMLV pol gene and comprises a stably integrated MoMLV gag and a gene encoding an Arena virus glycoprotein, including, but not limited to a Lassa glycoprotein. In another embodiment, said Arena virus glycoprotein gene (such as Lassa virus glycoprotein gene) is transiently expressed in the host cell. In another embodiment, said Arena virus glycoprotein gene (such as Lassa virus glycoprotein gene) is stably integrated into the cell genome. In another embodiment, the invention includes a cell line which lacks a replicon and pol gene and consists essentially of a stably integrated MoMLV gag gene and a gene encoding an Arena virus glycoprotein (such as a Lassa virus glycoprotein). In another embodiment, the invention includes a cell line which lacks a replicon and pol gene and consists of a stably integrated MoMLV gag gene and a gene encoding an Arena virus glycoprotein (such as a Lassa virus glycoprotein). In another embodiment, the invention includes a cell line which lacks a replicon and pol gene and consists of a stably integrated MoMLV gag gene, a stably integrated α(1,3) galactosyltransferase gene and a gene encoding an Arena virus glycoprotein (such as a Lassa virus glycoprotein).

In another embodiment, the invention includes a cell line which lacks a replicon and a pol gene and comprises a stably integrated MoMLV gag gene and a gene encoding a filovirus glycoprotein, including, but not limited to an Ebola virus glycoprotein or Marburg virus glycoprotein. In another embodiment, said filovirus glycoprotein gene (such as an Ebola virus glycoprotein gene or Marburg virus glycoprotein gene) is transiently expressed in the host cell. In another embodiment, said filovirus glycoprotein gene (such as an Ebola virus glycoprotein gene or Marburg virus glycoprotein gene) is stably integrated into the cell genome. In another embodiment, the invention includes a cell line which lacks a replicon and pol gene and consists essentially of a stably integrated MoMLV gag gene and a filovirus glycoprotein gene (such as an Ebola virus glycoprotein gene or Marburg virus glycoprotein gene). In another embodiment, the invention includes a cell line which lacks a replicon and pol gene and consists of a stably integrated MoMLV gag gene and a filovirus glycoprotein gene (such as an Ebola virus glycoprotein gene or Marburg virus glycoprotein gene). In another embodiment, the invention includes a cell line which lacks a replicon and pol gene and consists of a stably integrated MoMLV gag gene, a stably integrated α(1,3) galactosyltransferase gene and a filovirus glycoprotein gene (such as an Ebola virus glycoprotein gene or Marburg virus glycoprotein gene).

In one embodiment, the invention includes a cell line which lacks a replicon apol gene and comprises a stably integrated MoMLV gag gene and a gene encoding a bunyavirus glycoprotein, including, but not limited to a Rift Valley fever virus glycoprotein or Crimean Congo hemorrhagic fever virus glycoprotein. In another embodiment, said bunyavirus glycoprotein gene (such as Rift Valley fever virus glycoprotein gene or Crimean Congo hemorrhagic fever virus glycoprotein gene) is transiently expressed in the host cell. In another embodiment, said bunyavirus glycoprotein gene (such as Rift Valley fever virus glycoprotein gene or Crimean Congo hemorrhagic fever virus glycoprotein gene) is stably integrated into the cell genome. In another embodiment, the invention includes a cell line which lacks a replicon and pol gene and consists essentially of a stably integrated MoMLV gag gene and a gene encoding a bunyavirus glycoprotein (such as Rift Valley fever virus glycoprotein gene or Crimean Congo hemorrhagic fever virus glycoprotein gene). In another embodiment, the invention includes a cell line which lacks a replicon and pol gene and which consists of a stably integrated MoMLV gag gene and a gene encoding a bunyavirus glycoprotein (such as Rift Valley fever virus glycoprotein gene or Crimean Congo hemorrhagic fever virus glycoprotein gene). In another embodiment, the invention includes a cell line which lacks a replicon and a pol gene and which consists of a stably integrated MoMLV gag gene, a stably integrated α(1,3) galactosyltransferase gene and a gene encoding a bunyavirus glycoprotein (such as Rift Valley fever virus glycoprotein gene or Crimean Congo hemorrhagic fever virus glycoprotein gene).

The invention also encompasses variants of the said viral glycoproteins expressed on or in the cells and pseudovirons, including but not limited to replicon-deficient viral particles, of the invention. The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

Natural variants can occur due to antigenic drifts. Antigenic drifts are small changes in the viral proteins that happen continually over time. Thus, a person infected with a viral strain develops antibody against that virus, as newer virus strains appear, the antibodies against the older strains no longer recognize the newer virus and reinfection can occur. The invention comprises cloning and expressing these natural variant glycoproteins of the viruses mentioned above. More than one variant of a particular virus may be expressed in the cell lines of the invention.

In some embodiments of the invention, the heterologous viral glycoproteins expressed on the surfaces of the pseudovirions are chimeric glycoproteins. As used herein, the term “chimeric glycoproteins” refers to glycoproteins that contain domains from two or more glycoproteins from different viruses. By way of example, the cytoplasmic domain of a glycoprotein from virus A can be replaced with the cytoplasmic domain of a glycoprotein from virus B creating a chimeric glycoprotein. One or more domains, such as the cytoplasmic domain, the transmembrane domain, or the extracellular domain, from a particular viral glycoprotein may be replaced by a corresponding domain from a second viral glycoprotein. Upon expression in a host cell, such chimeric glycoproteins may be localized to different cellular compartments as compared to the native glycoproteins (see Examples 7 and 11). For example, bunyaviral glycoproteins localize to the Golgi apparatus. A chimeric glycoprotein, in which the cytoplasmic domain (or both the cytoplasmic and transmembrane domains) of a bunyaviral glycoprotein is replaced with the corresponding domain from the MoMLV envelope protein, localizes to the plasma membrane. Such re-localization of chimeric glycoproteins may be advantageous for increasing the overall amount of glycoproteins on the surface of pseudovirions budding from the plasma membrane. In one embodiment, the chimeric glycoprotein contains an extracellular and transmembrane domain of a Rift Valley Fever Virus glycoprotein and a cytoplasmic domain from the MoMLV envelope protein. In another embodiment, the chimeric glycoprotein contains an extracellular domain of a Rift Valley fever virus glycoprotein and a transmembrane and cytoplasmic domain from the MoMLV envelope protein.

In other embodiments of the invention, the gene encoding the heterologous viral glycoprotein is codon-optimized for expression in mammalian cells. Codon-optimization refers to a process in which a nucleotide sequence encoding a protein is altered to favor the codon frequency of the host expression system in which expression of the protein is desired. Different organisms utilize variations of the genetic code in which some codons are used more frequently than others. This phenomenon, known as codon bias, sometimes makes protein expression in heterologous systems difficult. These problems are particularly evident between prokaryotes and eukaryotes, i.e. when a prokaryotic protein is expressed in a eukaryotic host or vice versa. By altering the nucleotide sequence encoding the protein of interest to match the codon bias of the desired host cell, expression of the protein can be significantly improved. As such, codon-optimizing enveloped virus genes encoding glycoproteins for expression in mammalian cells is contemplated by the present invention. The codon-optimized viral glycoprotein genes can be used in the novel packaging cell lines described herein as well as methods of making pseudovirus and vaccine preparations. Methods of optimizing codons for different expression systems are known in the art (see, e.g., Babcock et al., (2004), J. Virology, Vol. 78:4552-4560) as well as various programs for designing a codon-optimized sequence, such as OptimumGene™ from GenScript and codon optimization calculator from Encor Biotechnology Inc. Codon-optimized gene sequences typically encode the same amino acid sequence as the native gene sequence.

In one embodiment of the invention, the codon-optimized viral glycoprotein gene is an arenavirus glycoprotein gene. In another embodiment, the arenavirus glycoprotein gene is a lassa virus glycoprotein gene. In another embodiment, the codon-optimized viral glycoprotein gene is a filovirus glycoprotein gene. In yet another embodiment, the filovirus glycoprotein gene is a Marburg virus glycoprotein gene or Ebola virus glycoprotein gene. In still another embodiment, the codon-optimized viral glycoprotein gene is a bunyavirus glycoprotein gene. In another embodiment, the bunyavirus glycoprotein gene is a Rift Valley Fever virus glycoprotein gene or a Crimean-Congo hemorrhagic fever virus glycoprotein gene.

The present invention also provides an isolated nucleotide encoding a Rift Valley Fever virus glycoprotein, wherein said isolated nucleotide is codon-optimized for expression in a mammalian cell. In one embodiment, the isolated polynucleotide comprises a sequence of SEQ ID NO: 2. In another embodiment, the present invention provides an isolated nucleotide encoding a Lassa virus glycoprotein, wherein said isolated nucleotide is codon-optimized for expression in a mammalian cell. In another embodiment, the isolated polynucleotide comprises a sequence of SEQ ID NO: 5. In another embodiment, the invention provides an expression vector comprising an isolated polynucleotide sequence encoding the Rift Valley Fever or Lassa virus glycoprotein. In still another embodiment, the isolated polynucleotide produces a functional (e.g. properly localized) Rift Valley Fever virus glycoprotein or Lassa virus when expressed in a mammalian cell.

General texts which describe molecular biological techniques, which are applicable to the present invention, such as cloning, mutation, creation of chimeric genes, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutation viral glycoproteins. Thus, the invention also encompasses using known methods of protein engineering and recombinant DNA technology to improve or alter the characteristics of the viral glycoprotein (such as creation of chimeric glycoproteins) expressed on or in the cell and pseudovirons of the invention. Various types of mutagenesis can be used to produce and/or isolate variant viral glycoproteins. They include, but are not limited to, site-directed mutagenesis, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like.

Methods of cloning said viral glycoproteins are known in the art. For example, the retroviral genes encoding glycoproteins can be isolated by RT-PCR from polyadenylated mRNA extracted from cells which had been infected with a virus. The resulting product gene can be cloned as a DNA insert into a vector. The term “vector” refers to the means by which a nucleic acid can be propagated and/or transferred between organisms, cells, or cellular components. Vectors include plasmids, viruses, defective viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all embodiments, the vectors of the present invention are plasmids or defective viruses.

Thus, the invention comprises nucleotides which encode at least one viral glycoprotein cloned into an expression vector which can be expressed in a cell and incorporated into pseudovirons of the invention. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. The promoter can also be an inducible promoter.

After the nucleotides encoding said viral glycoproteins have been cloned said nucleotides can be further manipulated. For example, a person with skill in the art can mutate specific bases in the coding region to produce variants. The variants may contain alterations in the coding regions, non-coding regions, or both. Such variants may increase the immunogenicity of a viral glycoprotein or remove a splice site from a protein or RNA.

In some embodiments, mutations containing alterations which produce silent substitutions, additions, or deletions, but do not alter the properties or activities of the encoded glycoprotein or how the glycoproteins are made. Nucleotide variants can be produced for a variety of reasons, e.g., to optimize codon expression for a particular host. In addition, the nucleotides can be sequenced to ensure that the correct coding regions were cloned and do not contain any unwanted mutations. The nucleotides can be subcloned into an expression vector for expression in any cell. The expression constructs will further contain sites for transcription initiation, termination, and, in the transcribed region, a ribosome binding site for translation. The coding portion of the transcripts expressed by the constructs will preferably include a translation initiating codon at the beginning and a termination codon appropriately positioned at the end of the polypeptide to be translated.

The expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBacl pWINEO, pSV2CAT, pOG44, pXT1 and pSG, pSVK3, pBPV, pMSG, and pSVL. Other suitable vectors will be readily apparent to the skilled artisan.

Next, the recombinant vector can be transfected, infected, or transformed into a suitable host cell of the invention. The host cell may comprise a MoMLV gag gene, pol gene and a replication-defective genome. Thus, the invention provides for host cells which comprise a vector (or vectors) that contain nucleic acids which code for at least one viral glycoprotein and can make MoMLV viral particles comprising said viral glycoproteins under conditions which allow the formation of MoMLV pseudovirons.

In another embodiment, the host cell may comprise a MoMLV gag gene and pol gene but no replicon. In yet another embodiment, the host cell may comprise a MoMLV gag gene but no pol gene or replicon. Thus, the invention provides for host cells which comprise a vector (or vectors) that contain nucleic acids which code for at least one viral glycoprotein and can make MoMLV replicon-deficient viral particles.

Non-limiting examples of host cells include, but are not limited to, COS cells, baby hamster kidney cells, mouse L cells, LNCaP cells, Chinese hamster ovary (CHO) cells, human embryonic kidney (HEK) cells, African green monkey cells, CV1 cells, HeLa cells, MDCK cells, Vero and Hep-2 cells. In one embodiment, said cells comprising MoMLV gag, pol, replication-defective genome and a viral glycoprotein are HEK 293 cells. In another embodiment, said HEK 293 cells comprise pGP-IRES-ZEO, pLEIN-IRES-NEO and a vector comprising at least one heterologous viral glycoprotein. In another embodiment, said pGP-IRES-ZEO, pLEIN-IRES-NEO are stably integrated into the cell genome. In another embodiment, said vector comprising at least one heterologous viral glycoprotein is stably integrated into the cell genome.

Vectors, e.g., vectors comprising MoMLV gag and pol, a replication-defective genome and viral glycoprotein polynucleotides, or vectors lacking a replicon comprising MoMLV gag and pol and viral glycoprotein polynucleotides or MoMLV gag (no pol) and viral glycoprotein polynucleotides can be transfected into host cells according to methods well known in the art. For example, introducing nucleic acids into eukaryotic cells can be by calcium phosphate co-precipitation, electroporation, microinjection, lipofection, and transfection employing polyamine transfection reagents, or any other procedure known in the art.

In another embodiment, said packaging system is composed of a cell line (e.g. Human embryonic Kidney (HEK) 293 cells, CV1 cells, HeLa cells, MDCK cells, Vero and Hep-2 cells), MoMLV gag and pol, a MoMLV viral replication-defective genome stably integrated into its genome and at least one viral glycoprotein of interest. In another embodiment, said replication-defective genome comprises a reporter gene, such as Enhanced Green Fluorescent Protein (EGFP) gene or choline acetyltransferase (CAT) gene. The gene encoding a viral surface glycoprotein, usually on a standard mammalian expression vector introduced by transient transfection, yields infectious but replication-incompetent pseudovirions with a MoMLV backbone (RNA, nucleoproteins, etc.) and the viral glycoprotein of choice. In another embodiment, said gene encoding a viral surface glycoprotein is stably incorporated into cell genome. In some embodiments, the viral surface glycoprotein is expressed from an inducible promoter.

Another embodiment of the invention comprises a cell line which comprises MoMLV gag, pol, a replication-defective genome and mouse α(1,3) galactosyltransferase gene stably integrated into a host cell genome. In another embodiment, the invention includes a cell line without a replicon that comprises MoMLV gag and pol and mouse α(1,3) galactosyltransferase gene stably integrated into a host cell genome. In yet another embodiment, the invention includes a cell line without a replicon or pol gene that comprises MoMLV gag and mouse α(1,3) galactosyltransferase gene stably integrated into a host cell genome. The α(1,3) galactosyltransferase enzyme catalyzes the synthesis of αGalactosyl (αGal) epitopes in the Golgi apparatus of cells from various non-primate mammals. The enzyme was found to be active in new world monkeys but not in old world monkeys and humans. Old world monkeys and humans do not encode a functional α(1,3) galactosyltransferase gene. Thus, the αGal epitope is a foreign epitope in old world monkeys and humans. In fact, anti-αGal antibodies are present in all humans. These antibodies specifically interact with the carbohydrate epitope Gal α(1-3), Gal β(1,4) GlcNAc-R (αGal epitope). These antibodies do not interact with any other known carbohydrate epitope produced by mammalian cells (Galili, 1993, Springer Seminar Immunopathology 15:153). Anti-αGal antibodies constitute approximately 1% of circulating IgG in humans (Galili et al., 1984, J. Exp. Med. 160:1519) and are also found in the form of IgA and IgM (Davine et al., 1987, Kidney Int. 31:1132; Sandrin et al., 1993, Proc. Natl. Acad. Sci. USA 90:11391). It is thought that humans are exposed to the αGal epitope from intestinal flora. The αGal epitope causes opsonization (via binding of anti-α gal antibodies) thereby enhancing uptake of the proteins by antigen presenting cells which results in enhanced antigen presentation. The animal's immune system is then stimulated to produce specific cytotoxic cells and antibodies which will potentiate a specific immune response toward the protein. (See co-pending application U.S. 2007/0014775 and U.S. Pat. No. 5,879,675, herein incorporated by reference in their entireties for all purposes.)

Thus, a protein comprising the αGal epitope can immunopotentiate the immune system of an old world money and/or human against said protein. Thus, one embodiment of the invention comprises a MoMLV pseudovirion comprising an αGal epitope. In another embodiment, the invention comprises a cell line comprising, a stably integrated MoMLV gag, a stably integrated MoMLV pol, a stably integrated MoMLV replication-defective genome, a stably integrated a (1,3) galactosyltransferase gene (see U.S. Pat. No. 5,879,675) and at least one heterologous virus glycoprotein gene from an enveloped virus. The a (1,3) galactosyltransferase gene may be from any species except humans, Old World monkeys, birds, and other species with a non-functional gene. In one embodiment, the α(1,3) galactosyltransferase gene is a mouse α(1,3) galactosyltransferase gene. In another embodiment, said glycoprotein gene from an enveloped virus is stably integrated into the cell line. In another embodiment, said glycoprotein gene from an enveloped virus is from an arenavirus. In another embodiment, said arenavirus is a Lassa virus. In another embodiment, said glycoprotein gene from an enveloped virus is from a filovirus. In another embodiment, said filovirus is an Ebola virus. In another embodiment, said filovirus is a Marburg virus. In another embodiment, said glycoprotein is bunyavirus. In one embodiment, the bunyavirus is Rift Valley fever virus. In another embodiment, the bunyavirus is Crimean Congo hemorrhagic fever virus. In another embodiment, said cell line comprises glycoprotein genes from different enveloped viruses. In another embodiment, said cell line comprises a glycoprotein gene from a BSL 4 classified enveloped virus. In another embodiment, the glycoprotein gene is a chimeric glycoprotein gene. In another embodiment, said cell line generates a titer of virus particles (e.g., pseudovirions or replicon-deficient virus particles) of at least about 1.0×10⁵ cfu/ml, about 5.0×10⁵ cfu/ml, about 7.0×10⁵ cfu/ml, about 9.0×10⁵ cfu/ml or about 1.0×10⁶ cfu/ml. In another embodiment, said cell line is derived from an old world monkey. In another embodiment, said cell line is derived from a human. In another embodiment, said cell line is human embryonic kidney (HEK) 293 cells. In another embodiment, said αGal is chemically and/or enzymatically added to the proteins on the pseudovirions. Methods of chemically and/or enzymatically adding αGal epitopes to the proteins are known in the art. One method is by utilizing the enzyme α-1,3 galactosyltransferase (α-1,3 GT). Recombinant α 1,3GT which may be used in the reaction can been obtained from several different species including from New World monkeys (see Example 8 and U.S. Pat. No. 5,879,675, herein incorporated by reference).

The present invention also encompasses a cell line comprising, a stably integrated MoMLV gag, a stably integrated MoMLV pol, a stably integrated MoMLV replication-defective genome, at least one gene encoding a heterologous viral glycoprotein, and at least one gene encoding a heterologous viral nucleoprotein. In one embodiment of the invention, the cell line produces replicon-deficient viral particles, i.e., the cell line does not contain a viral replicon. In another embodiment, the cell line does not contain a MoMLV replicon. In another embodiment, the cell line does not contain a viral pol gene. In another embodiment, the cell line does not contain a MoMLV pol gene. In yet another embodiment, the cell line does not contain a pol gene or replicon.

The gene encoding a viral nucleoprotein may be from the same virus as the gene encoding a viral glycoprotein. In one embodiment, the gene encoding a viral glycoprotein and the gene encoding the viral nucleoprotein are Rift Valley Fever virus genes. The gene encoding a viral nucleoprotein may be from a different virus than the gene encoding a viral glycoprotein.

In another embodiment, said gene encoding the heterologous viral nucleoprotein is transiently expressed in the host cell. In another embodiment, said heterologous viral nucleoprotein is stably integrated into the cell genome. In some embodiments, the heterologous viral nucleoprotein may be expressed from an inducible promoter.

### Formulations, Administration and Methods of Making Virus Particles of the Invention

The invention also comprises antigenic formulations and/or vaccines for inducing an immune response to protect against viral infections. The antigenic formualations and/or vaccine comprise pseudovirus produced from the cell lines of the invention as described herein. In one embodiment, the invention comprises an antigenic and/or vaccine preparation against a specific virus comprising, a MoMLV replication-defective genome, MoMLV protease, reverse transcriptase, integrase, capsid and nucleocapsid proteins and at least one virus surface glycoprotein. In another embodiment, said virus surface glycoprotein is from an enveloped virus. In another embodiment, said glycoprotein gene from an enveloped virus is from an arenavirus. In another embodiment, said arenavirus is a Lassa virus. In another embodiment, said glycoprotein gene from an enveloped virus is from a filovirus. In another embodiment, said filovirus is an Ebola virus. In another embodiment, said filovirus is a Marburg virus. In another embodiment, said glycoprotein is bunyavirus. In one embodiment, the bunyavirus is Rift Valley fever virus. In another embodiment, the bunyavirus is Crimean Congo hemorrhagic fever virus. In another embodiment, said cell line comprises glycoprotein genes from different enveloped viruses. In another embodiment, said cell line comprises a glycoprotein gene from a BSL-4 classified enveloped virus. In another embodiment, said cell line generates a titer of virus particles (e.g., pseudovirions or replicon-deficient virus particles) of at least about 1.0×10⁵ cfu/ml, at least about 5.0×10⁵ cfu/ml, at least about 7.0×10⁵ cfu/ml, at least about 9.0×10⁵ cfu/ml or at least about 1.0×10⁶ cfu/ml.

In preferred embodiments, the antigenic and/or vaccine preparation comprises replicon-deficient viral particles produced by the cell packaging lines described herein. In one embodiment, the antigenic and/or vaccine preparation comprises a MoMLV protease, reverse transcriptase, integrase, capsid and nucleocapsid proteins and at least one viral glycoprotein. In another embodiment, said virus surface glycoprotein is from an enveloped virus. In another embodiment, said glycoprotein gene from an enveloped virus is from an arenavirus. In another embodiment, said arenavirus is a Lassa virus. In another embodiment, said glycoprotein gene from an enveloped virus is from a filovirus. In another embodiment, said filovirus is an Ebola virus. In another embodiment, said filovirus is a Marburg virus. In another embodiment, said glycoprotein is from a bunyavirus. In one embodiment, the bunyavirus is Rift Valley fever virus. In another embodiment, the bunyavirus is Crimean Congo hemorrhagic fever virus. In another embodiment, the viral glycoprotein is a chimeric glycoprotein. In another embodiment, said cell line comprises glycoprotein genes from different enveloped viruses. In another embodiment, said cell line comprises a glycoprotein gene from a BSL-4 classified enveloped virus. In another embodiment, said glycoprotein gene is codon-optimized for expression in a mammalian cell. In another embodiment, said cell line generates a titer of virus particles (e.g., pseudovirions or replicon-deficient virus particles) of at least about 1.0×10⁵ cfu/ml, at least about 5.0×10⁵ cfu/ml, at least about 7.0×10⁵ cfu/ml, at least about 9.0×10⁵ cfu/ml or at least about 1.0×10⁶ cfu/ml. In still another embodiment, said cell line further comprises a nucleoprotein gene from a heterologous virus.

The antigenic and/or vaccine compositions contain a pharmaceutically acceptable carrier, including any suitable diluent or excipient, which includes any pharmaceutical agent that does not itself induce the production of an immune response harmful to the vertebrate receiving the composition, and which may be administered without undue toxicity, and a pseudovirion of the invention. As used herein, the term “pharmaceutically acceptable” means being approved by a regulatory agency of the Federal or a state government or listed in the U.S. Pharmacopia, European Pharmacopia or other generally recognized pharmacopia for use in vertebrates, and more particularly in humans. These compositions can be useful as a vaccine and/or antigenic compositions for inducing a protective immune response in a vertebrate. A “protective immune response” as used herein refers to an immune response against an infectious agent (e.g. a virus), which is exhibited by a vertebrate (e.g., a human), that prevents or ameliorates an infection or reduces at least one symptom thereof. Such compositions can be administered to a subject intranasally, intradermally, intramuscularly, intravenously and/or subcutaneously.

In some embodiments, said pharmaceutical formulations of the invention comprise pseudovirions or viral particles comprising a MoMLV protease, reverse transcriptase, integrase, capsid and nucleocapsid proteins, at least one heterologous virus surface glycoprotein protein and a pharmaceutically acceptable carrier or excipient. In other embodiments, said pharmaceutical formulations of the invention comprise pseudovirions or viral particles comprising MoMLV gag proteins and at least one heterologous virus surface glycoprotein protein and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The invention also provides for a pharmaceutical pack or kit comprising one or more containers filled with one or more of the ingredients of the vaccine formulations of the invention. In one embodiment, the kit comprises two containers, one containing virus particles (e.g., pseudovirions, virus-like particles, or replicon-deficient virus particles) and the other containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that the virus particle (e.g., pseudovirions, virus-like particles, or replicon-deficient virus particles) formulation be packaged in a hermetically sealed container such as an ampoule or sachette indicating the quantity of composition. In one embodiment, the virus particle (e.g., pseudovirions, virus-like particles, or replicon-deficient virus particles) composition is supplied as a liquid, in another embodiment, as a dry sterilized lyophilized powder or water free concentrate in a hermetically sealed container and can be reconstituted, e.g., with water or saline to the appropriate concentration for administration to a subject. In another embodiment, the virus particle (e.g., pseudovirions, virus-like particles, or replicon-deficient virus particles) composition is supplied as a dry sterile lyophilized powder in a hermetically sealed container at a unit dosage of about 1 μg, about 5 μg, about 10 μg, about 20 μg, about 25 μg, about 30 μg, about 50 μg, about 100 μg, about 125 μg, about 150 μg, or about 200 μg. Alternatively, the unit dosage of the virus particle (e.g., pseudovirions or replicon-deficient virus particles) composition is less than about 1 μg, (for example about 0.08 μg, about 0.04 μg, about 0.2 μg, about 0.4 μg, about 0.8 μg, about 0.5 μg or less, about 0.25 μg or less, or about 0.1 μg or less), or more than about 125 μg, (for example about 150 μg or more, about 250 μg or more, or about 500 μg or more). These doses may be measured as total virus particles (e.g., pseudovirions, virus-like particles, or replicon-deficient virus particles) or as μg of heterologous viral glycoprotein (e.g., Rift Valley fever virus, Crimean Congo hemorrhagic fever virus, Lassa virus or Ebola virus glycoprotein). The virus particle (e.g., pseudovirions, virus-like particles, or replicon-deficient virus particles) composition should be administered within about 12 hours, preferably within about 6 hours, within about 5 hours, within about 3 hours, or within about 1 hour after being reconstituted from the lyophylized powder.

In an alternative embodiment, a virus particle (e.g., pseudovirions, virus-like particles, or replicon-deficient virus particles) composition is supplied in liquid form in a hermetically sealed container indicating the quantity and concentration of the virus particle composition. Preferably, the liquid form of the virus particle composition is supplied in a hermetically sealed container at least about 50 μg/ml, at least about 100 μg/ml, at least about 200 μg/ml, at least 500 μg/ml, or at least 1 mg/ml of total virus particles or total heterologous viral glycoprotein.

Generally, virus particles of the invention are administered in an effective amount or quantity sufficient to stimulate an immune response against one or more viruses. Preferably, administration of the virus particles of the invention elicits immunity against a viral infection. Typically, the dose can be adjusted based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by liquid drops, large particle aerosol (greater than about 10 microns), or spray into the upper respiratory tract.

Methods of administering a composition comprising virus particles (vaccine and/or antigenic formulations) include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions of the present invention are administered intramuscularly, intravenously, subcutaneously, transdermally or intradermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition comprising virus particles of the invention may induce an antibody or other immune response that is substantially higher than other routes of administration. In another embodiment, intranasal or other mucosal routes of administration of a composition comprising virus particles of the invention may induce an antibody or other immune response that will induce protection against viruses. Administration can be systemic or local.

In another embodiment, said virus particles of the invention can be administered as part of a combination therapy. For example, virus particles of the invention can be formulated with other immunogenic compositions and/or antivirals (e.g. Amantadine, Rimantadine, Zanamivir and Osteltamivir).

The dosage of the pharmaceutical formulation can be determined readily by the skilled artisan, for example, by first identifying doses effective to elicit a prophylactic or therapeutic immune response, e.g., by measuring the serum titer of viral specific immunoglobulins or by measuring the inhibitory ratio of antibodies in serum samples, or urine samples, or mucosal secretions. Said dosages can be determined from animal studies. A non-limiting list of animals used to study the viral vaccines includes the guinea pig, Syrian hamster, chinchilla, hedgehog, chicken, rat, mouse and ferret. Most animals are not natural hosts to specific viruses but can still serve in studies of various aspects of the disease. In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

As also well known in the art, the immunogenicity of a particular composition can be enhanced by the use of non-specific stimulators of the immune response, known as adjuvants. Adjuvants have been used experimentally to promote a generalized increase in immunity against unknown antigens. Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2nd Edition),” herein incorporated by reference in its entirety for all purposes, is envisioned within the scope of this invention. For instance, suitable adjuvants include, but are not limited to Freund's adjuvant (a non-specific stimulator of the immune response containing killed Mycobacterium tuberculosis), incomplete Freund's adjuvants, aluminum hydroxide (alum), CpG-containing oligonucleotides, saponins (e.g. QS21), GMCSP, BCG, MDP compounds, such as thur-MDP and nor-MDP, CGP (MTP-PE), lipid A, monophosphoryl lipid A (MPL), RIBI (which contains three components extracted from bacteria: MPL, trehalose dimycolate (TDM) and cell wall skeleton (CWS) in a 2% squalene/Tween 80 emulsion), MF-59, Novasomes®, MHC antigens, and commercially available adjuvant systems, such as Sigma Adjuvant System™. In one embodiment of the invention, the vaccine preparation further comprises an adjuvant.

Another embodiment of the invention comprises a method of preparing antigenic formulations and/or vaccines against a virus comprising, transfecting a heterologous virus glycoprotein gene into the cell lines described above, collection and concentration of the virus particles (i.e., pseudovirions or replicon-deficient virus particles), and resuspension in a pharmaceutically acceptable buffer for injection. In one embodiment, said heterologous virus glycoprotein gene is from Lassa virus. In another embodiment, said heterologous virus glycoprotein gene is from Ebola hemorrhagic fever virus. In another embodiment, said glycoprotein is bunyavirus. In one embodiment, the bunyavirus is Rift Valley fever virus. In another embodiment, the bunyavirus is Crimean Congo hemorrhagic fever virus. In another embodiment, said pseudovirions comprise α-gal epitopes. In another embodiment, said glycoprotein is a chimeric glycoprotein.

Methods for making and purifying the virus particles (i.e., pseudovirions or replicon-deficient virus particles) of the invention are known in the art. Methods to grow cells engineered to produce virus particles of the invention include, but are not limited to, batch, batch-fed, continuous and perfusion cell culture techniques. Cell culture means the growth and propagation of cells in a bioreactor (a fermentation chamber) where cells propagate and release virus particles for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, said bioreactor is a stainless steel chamber. In another embodiment, said bioreactor is a pre-sterilized plastic bag (e.g. Cellbag®, Wave Biotech, Bridgewater, N.J.). The virus particles are then isolated using methods that preserve the integrity thereof, such as by gradient centrifugation, e.g., cesium chloride, sucrose and iodixanol, as well as standard purification techniques including, e.g., ion exchange and gel filtration chromatography.

This invention is further illustrated by the following examples that should not be construed as limiting. The contents of all references, patents and published patent applications cited throughout this application, as well as the Figures are incorporated herein by reference.

### EXAMPLES

### Example 1

### Making of pGP-IRES-Zeo

The plasmid containing Moloney Murine Leukemia Virus (MoMLV)-based helper virus, pPAM3 (Fred Hutchinson Cancer Research Center, Seattle, Wash.), was digested by AflIII to remove the env gene, followed by Klenow treatment and self-ligation to generate pGP. A 2.8-kb DNA fragment consisting of the IRES-Zeo expression cassette, SV40 poly(A) signal, bacterial replication origin (ColE1 Ori), and phage replication origin (F1 Ori) was excised from pIRES-Zeo (Young, W. B. and C. J. Link, Jr., 2000) by Eagi digestion, subjected to Klenow treatment and that digested with XbaI. This 2.8-kb IRES-Zeo fragment was subsequently ligated into pGP to generate pGP-IRES-Zeo. The resulting chimeric helper virus plasmid, pGP-IRES-Zeo, allows selection with Zeocin in bacterial culture and mammalian cells.

### Example 2

### Making of pLEGFP-IRES-Neo

The LEIN retroviral vector carrying an EGFP reporter gene was constructed by replacing the SV40 promoter-neomycin phosphotransferase gene (Neo^{r}) cassette of pLESN (Mazo, I. A., et al., 1999) with a 1.4-kb IRES-Neo cassette, excised from pIRES-Neo (Clontech, Mountain View, Calif.) by Nael and NsiI digestions.

### Example 3

### Making of Packaging Cell Line

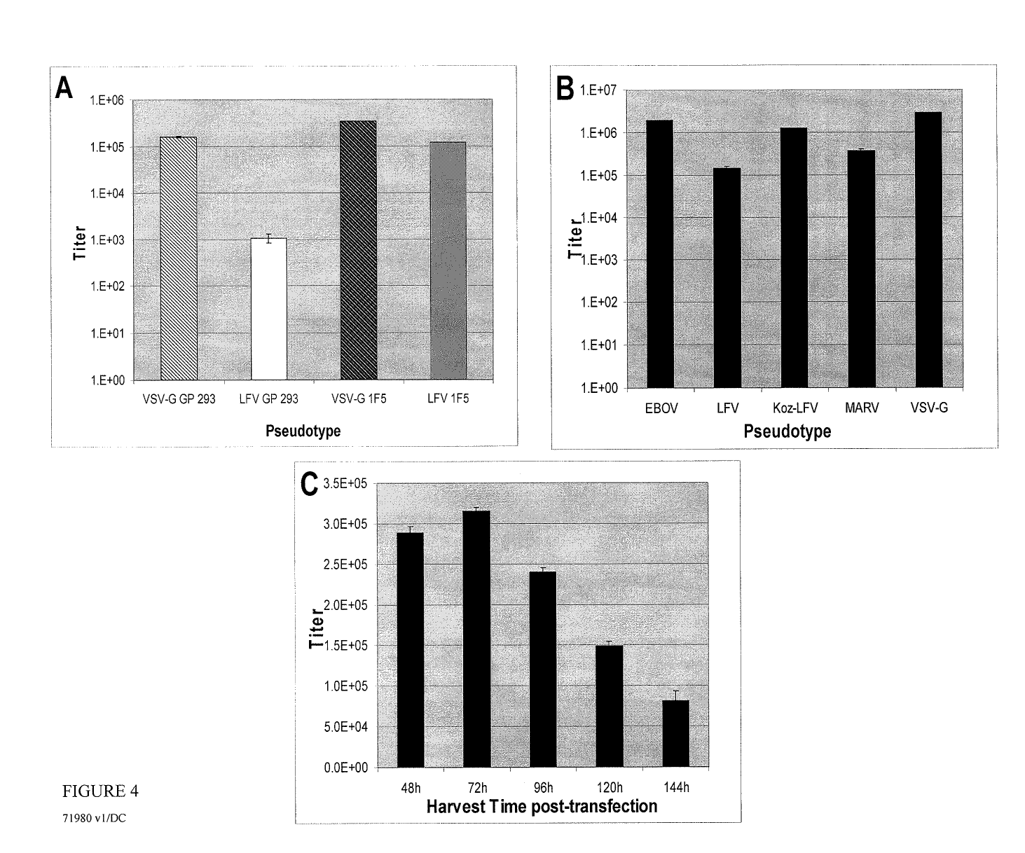

The pLEGFP-IRES-Neo (8.3 μg) replication-defective genome vector was linearized with ScaI and transfected into pGP-IRES-Zeo cells using a standard calcium phospate transfection protocol and reagents (37° C., 5% CO₂). Transfected GP293 cells were placed under G418 selection (DMEM, 10% FBS, 2 mM L-Glutamine, 0.6 mg/ml G418) 48 hours post transfection to select for those clones that had stably integrated the replication-defective genome. The selected clones were maintained under selective growth conditions (37° C., 5% CO₂). A single cell sort of those clones was performed. From 192 potential clones, 24 showed significant EGFP activity. High-throughput transient transfections of those 24 clones with the LV-GP expression plasmid pPreGPCcDNA3.1 were performed. Transductions of 293T cells using medium from these twenty-four transfected clones were then performed. From the original 24, two clones, pLEGFP-IRES-Neo GP293 1F5 and 2E6, were selected as prototype producer clones based on their ability to generate high-titer LV-pseudotyped virions (data below, FIG. 4). Titers were verified by performing the transductions two separate times, each in triplicate.

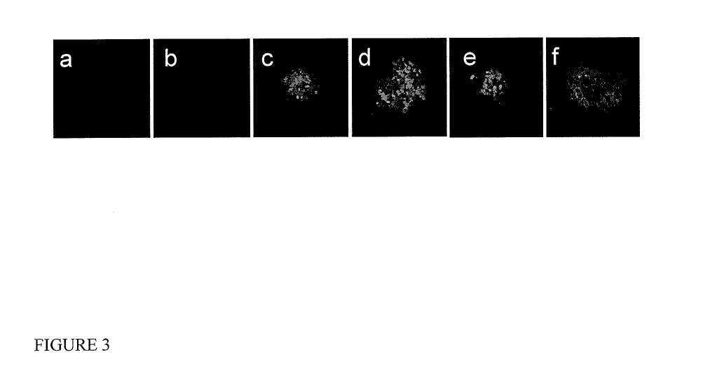

1F5 was transiently transfected with single plasmids encoding glycoproteins of the following viruses: EBOV, MARV, or LV using the standard calcium phospate (Invitrogen) transfection protocol and reagents. These transfected packaging cell lines were incubated at 37° C., 5% CO₂ for 3 days in DMEM, 10% FBS, 2 mM L-Glutamine. The media from these transfections were harvested and used to transduce Vero cells (37° C., 5% CO₂ for 20 hours in the presence of 10 μg/ml polybrene; medium changed at ˜20 h to fresh DMEM, 10% FBS, 2 mM L-Glutamine). The transduced cultures were analyzed using fluorescence microscopy approximately three days post-transduction (see FIG. 3).

### Example 4

### Testing of Packaging Cell Lines

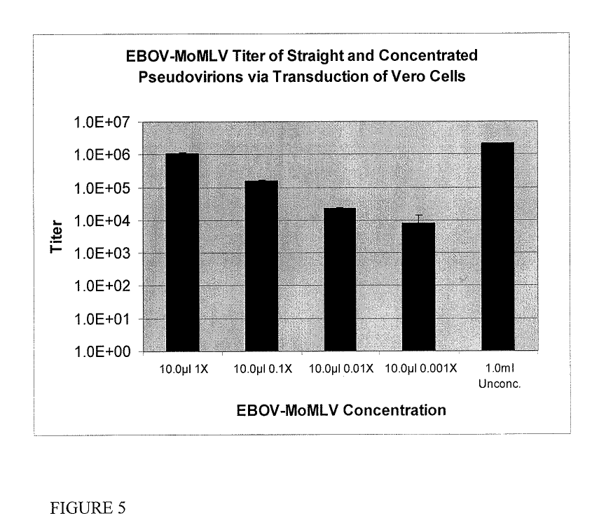

As described, the new MoMLV packaging cell lines, designated 1F5 and 2E6, incorporate the MoMLV replication-defective genome pLEGFP-IRES-Neo and gag/pol stably in their genome, and thus only require the addition (via transfection) of a single expression plasmid encoding the glycoprotein of interest to produce pseudovirions. 1F5 and 2E6 were transfected with vectors encoding EBOV-GP, MARV-GP or LV-GP to generate pseudovirions. Vero or 293 cells were transduced by exposure to pseudovirions in medium from transfected cells, and transduced cultures were analyzed using fluorescence microscopy (see FIG. 3) or FACS analysis (see FIGS. 4 and 5) to detect GFP expression (dependent upon generation of functional pseudovirions and successful transduction).

Producer clones were plated at 2×10⁶ cells per 100 mm plate, 24 hours prior to transfection. Expression vectors encoding the EBOV, LV and VSV (control) glycoproteins were transiently transfected into each clone using calcium phosphate. Cell culture supernatants containing pseudovirion particles were collected and filtered 72 hours post-transfection, and these were used to infect Vero and 293T cells (plated to six-well plates). Transductions were performed in triplicate and quantitated by FACS analysis. Experiments were performed to verify that the 1F5 (and 2E6, not shown) cell line generates more pseudovirions (higher titer) than the original (unmodified) GP293 Clontech MoMLV packaging cell line. LV-pseudotyped MoMLV titers were very low (1×10³ cfu/ml, FIG. 4A), such that the virion concentrations would not be sufficient for vaccine purposes. However, 1F5 was able to generate LV-pseudotyped MoMLV titers of greater than 1×10⁵ cfu/ml, which is at least a 100× increase. Interestingly, VSV G-pseudotyped MoMLV titers were over 1×10⁵ using either the two-vector transfection of GP293 cells or VSV GP-transfected 1F5, which illustrates that it is generally easier to generate VSV-MoMLV pseudovirions (and is why VSV-MoMLV was used as a control). Next MARV-pseudotyped MoMLV pseudovirions were produced (see FIG. 4B). Additionally, LV-pseudotyped MoMLV had increased titers, around 10×, by incorporating a Kozak DNA sequence immediately upstream of the ATG “start” codon in the LV-GP expression plasmid (Koz-LV-GP; see FIG. 4B). Finally, these data demonstrate that the optimal time for harvesting MoMLV pseudovirions is 72 h post-transfection (see FIG. 4C).

Transduction is the current measure of pseudovirion titer. Importantly, these results indicate that the pseudovirions produced are functional (can readily transduce (infect) cells), indicating a “natural” confirmation of the heterologous (EBOV, LV, VSV) viral glycoproteins in the MoMLV membrane, which is important for recognition and response by the immune system (efficacy of the vaccine). As shown, this has been confirmed by multiple experimental repetitions.

### Example 5

### Creating Additional Packaging Cell Lines

### BPSC-1 MoMLV Packaging Cell Lines (to Replace/Supercede 2E6)

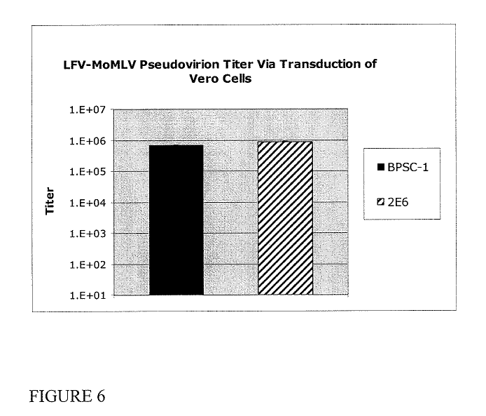

293 cells (ATCC CRL-1573, lot 5022670) containing stably integrated pGP-IRES-Zeo and the replication-defective genome pLEGFP-IRES-Neo (sequentially selected with Zeocin then Zeocin and neomycin) were expanded (double selection) and tested as described above (for 1F5) and below for the ability to generate MoMLV pseudovirions (compared to 2E6, a sister clone of 1F5). BPSC-1 was transiently transfected with an expression vector (pcDNA3.1+, Invitrogen) containing the LV glycoprotein gene(s) operably linked to the CMV promoter via the calcium phosphate method as described for 1F5, above. 2E6 was similarly treated as a positive control. Transfected BPSC-1 and 2E6 cells were cultured and LV-pseudotyped MoMLV was harvested from each as described above for 1F5. Generation of LV-pseudotyped MoMLV was measured by transfection of Vero cells as described above for 1F5. Cells were transduced in triplicate, and analyzed by FACS analysis for the expression of GFP encoded by the transduced replication-defective genome (pLEGFP-IRES-Neo). Results shown in FIG. 6.

GFP positive cells indicate a successful transduction of Vero cells via LV-pseudotyped MoMLV virions generated by BPSC-1 and 2E6 producer cell lines. These data indicate that BPSC-1 (mixed population) and 2E6 cells were able to generate titers of 6.9×10⁵ and 8.8×10⁵ cfu/ml, respectively. These results show that BPSC-1 packaging cell line works comparably to 1F5 and 2E6 (see above).

Importantly, the BPSC-1 producer cell line used to generate these data shown herein is a mixed population of cells, whereas 2E6 is a high titer clone selected through FACS sorting. Similar sorting by strength of GFP signal to isolate a single cell clone of BPSC-1 was also performed.

### αGal-Modifying MoMLV Packaging Cell Lines

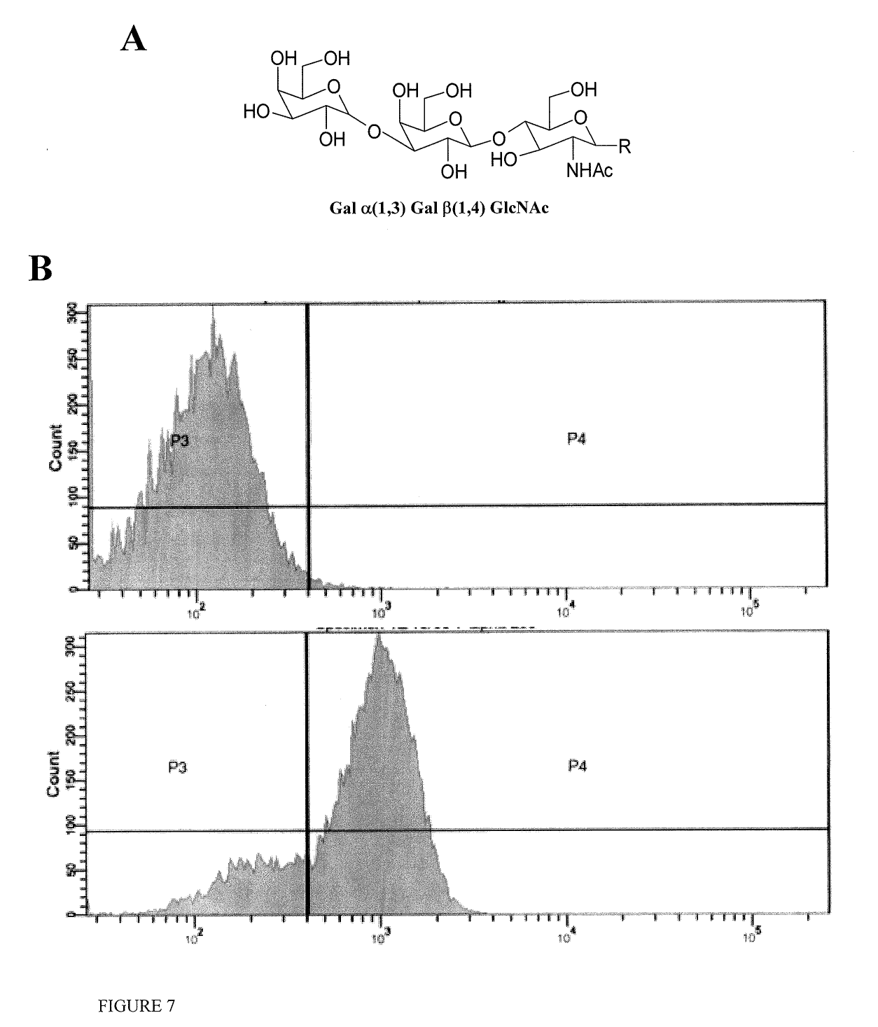

To biologically modify viral vaccine components based on pseudovirions and virus-like particles (VLPs) with αGal epitopes, stable MoMLV packaging cell lines that facilitate the budding of αGal(+) pseudovirus for use as antiviral vaccines were generated. The murine α1,3 Galactosyl Transferase (α1,3 GT) gene was stably integrated into packaging cells containing stably integrated MoMLV gag and pol genes, and a GFP-containing replication-defective genome. The integration of the α1,3 GT enabled the packaging cells to generate αGal epitopes (FIG. 7A) on their surface proteins. As shown in FIG. 7B, FACS analysis of clonal populations probed with an anti-αGal IgY indicates that >90% of the cells display surface αGal epitopes.

### Example 6

### Replicon-Deficient Viral Particles

Replicon-deficient viral particles can be produced by leaving out the replication-defective genome of the previously described MoMLV packaging cells. These cells, therefore, contain MoMLV gag and pol.

A cell line of replicon-deficient virions can also be created by integrating only gag (not pol) into 293 cells to generate a new producer line that will generate replicon-deficient viral particles. Transient transfection of a plasmid encoding a glycoprotein of interest will generate empty (RNA or replicon-deficient) pseudotyped MoMLV virus particles.

### Example 7

### Bunyaviral Pseudotyped MoMLV

The cell lines described in Example 6 can be used to produce Bunyaviral pseudotyped MoMLV. Both Rift Valley fever virus and Crimean Congo hemorrhagic fever virus have two surface glycoproteins encoded by one ORF (precursor molecule) on one RNA strand (m segment). These proteins will be expressed either as a precursor or as individual entities using mammalian expression vectors (e.g., pCAGGS and pcDNA3.1).

Bunyviral glycoproteins usually localize and are predominantly retained in the Golgi. Since MoMLV normally buds from the plasma membrane, it is necessary to facilitate localization of the glycoproteins to the producer cell plasma membrane. In order to facilitate the association of bunyaviral glycoproteins with MoMLV virus components (encoded by gag), chimeric forms of the bunyaviral glycoproteins are being developed in which a) cytoplasmic domains are replaced with MoMLV env cytoplasmic domains; b) cytoplasmic and transmembrane domains are replaced with MoMLV env transmembrane and cytoplasmic domains; and c) Golgi retention signals are rendered inactive by point mutations.

### Example 8

### Modification of Viral Proteins with αGal Epitopes

Biological addition of αGal epitopes is limited by the number of N-linked glycosylation sites present on surface proteins (vaccine components). Furthermore, not all N-linked carbohydrate chains are certain to be αGal-modified because of complexities (e.g., competing glycosyltransferases) inherent to mammalian glycosylation pathways. However, vaccine preparations (pseudovirions and VLPs) can be further modified with additional αGal epitopes using chemical or enzymatic modification protocols. Enzymatic addition adds αGal epitopes to existing N-linked sites and can insure that most N-linked chains are αGal modified. Chemical addition uses a succinimide or maleimide cross-linker attached to a “synthetic” αGal trisaccharide that couples to lysine or cysteine residues, respectively. This technique allows for the addition of substantially more αGal residues to most proteins, and thus potentially increases the potency of the αGal modified vaccines.

### Enzymatic Addition of αGal Epitopes

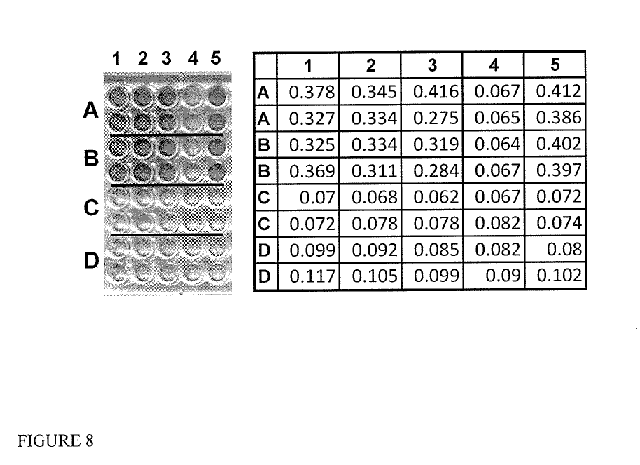

The ability to add αGal epitopes to purified viral vaccine components represents an important tool for placing or increasing the number of αGal epitopes on natural N-linked carbohydrate moieties. Marmoset α1,3 Galactosyltransferase (α1,3 GT) was cloned from the silvery marmoset (Callithrix argentata) NZP-60 (ATCC) cell line into the Pichia pastoris expression system (Invitrogen) and purified by Ni-column affinity chromatography. The activity of partially-purified α1,3 GT activity was confirmed by in vitro modification of fetuin (FIG. 8). ELISA plates were coated with 20 μg/ml fetuin (Sigma F2379) in carbonate/bicarbonate binding buffer (Sigma C3041) overnight at 4° C. Plates were washed 3× with a Tecan plate washer with PBS with 0.1% Tween-20. 50 μl of 5 μU/ml neuraminidase was added to some wells (as indicated) and incubated at 37° C. for 1 hour. Plates were washed as described above and the indicated reaction agents (5 μl purified α1,3 GT in 50 μl MES pH 6.0, 25 mM MnCl₂ with or without 5 μU/ml neuraminidase, and 1 mM uridine 5′-diphosphagalactose disodium (UDP-Gal, Sigma U4500)) were added and incubated at 37° C. for 2 hours. Plates were washed as described above, then 100 μl gelatine (Sigma G7765) in PBS with Tween-20 was added to each well and incubated at 37° C. for 1 hour. Plates were washed as described above, and then 50 μl of a 1:1000 dilution of HRP-conjugated rabbit anti-chicken IgG was added to each well and incubated at room temperature (RT) for 1 hour. Plates were washed 5× as described, and then 50 μl of a 1:1 mixture of peroxidase substrate (tetramethylbenzidine) and peroxide solution (Pierce, 34021) was added to the wells for 1 to 2 minutes at RT. The reaction was stopped by the addition of 50 μl 2M sulphuric acid (Fisher). Absorbance was determined at 450 nm (Multiskan Spectrum 1500, Thermo Labsystems).

The addition of αGal residues to substrate (fetuin) was dependent upon the presence of α1,3 GT (FIG. 8, lanes A and B) and UDP-Gal (FIG. 8, lanes A and B). Treatment of the substrate with neuraminidase did not affect activity (lane B). Purified enzyme was clearly active (row 1), and the presence of a denaturing agent (dithiothreitol) did not affect activity (rows 2 and 3). Purification of 1,3 GT under denaturing conditions inactivates the enzyme (row 4), but the enzyme can be re-activated if refolded (row 5). These data demonstrate that enzymatic addition of αGal epitopes by recombinant α1,3 GT is a feasible strategy to increase αGal content of antiviral vaccine components.

### Chemical Addition of αGal Epitopes

Chemical modification involves the addition of αGal epitopes to proteins using the αGal trisaccharide (FIG. 7A) coupled to a carbon chain linker (of a select length) and terminal maleimide or succinimide moieties for coupling to cysteine or lysine residues, respectively (Naicker et al. (2004) Org Biomol Chem., Vol. 2:660-664). Because there are usually many more available reactive sites on surface glycoproteins (terminal amines, etc) compared to the number of N-linked glycosylation sites (the natural template for biological and enzymatic αGal modification), chemical αGal addition leads to substantially more αGal epitopes on any particular protein compared to addition by other means (e.g., biologic or enzymatic). Additionally, the coupling reaction is very efficient.

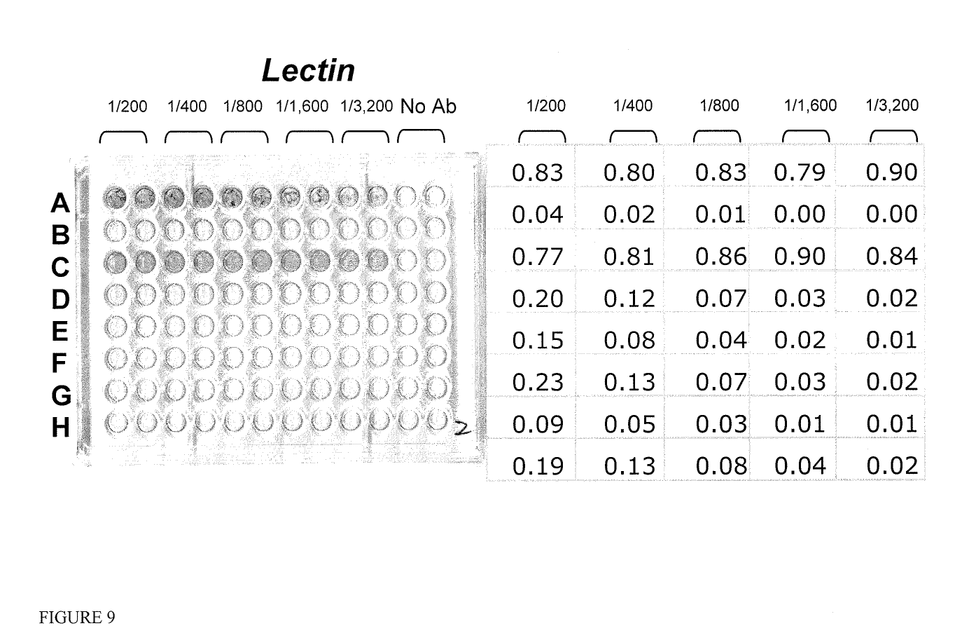

The chemical addition of αGal epitopes to antiviral vaccines is a good alternative to enzymatic addition, especially in situations where components are not naturally rich in N-linked carbohydrate chains. Chemical modification of peptides and especially of full-length glycoproteins (GPs) in a VLP and/or pseudovirion preparation will result in higher αGal levels compared to enzymatic treatment with the α1,3 GT enzyme (see FIG. 9). An ELISA-based assay format was used to compare the relative efficiency of αGal modification of Influenza hemagglutinin (Chiron's Influvirin Influenza hemagglutinins (HA) peptides (H1N1, H3N2, Influenza B)) by enzymatic and chemical means. Each well in a microtiter plate was coated with either 0.25 μg of Chiron's Influrin Influenza hemagglutinins (HA) peptides (H1N1, H3N2, Influenza B) or albumin according to the following key:
A: αGal-conjugated human serum albumin
B: human serum albumin
C: Influenza HAs from Chiron vaccine Influvirin chemically modified with αGal epitopes (Dextra Laboratories Limited)
D: HAs modified by α1,3 GT enzyme (Sigma-Aldrich)
E: HAs modified by NewLink α1,3 GT(1)
F: HAs modified by NewLink α1,3 GT(2)
G: HAs treated as in D, E, F but without α1,3 GT enzyme
H: untreated HAs

αGal modification was detected by serial dilutions of biotin-conjugated lectin from 1/200× (125 ng/50 μl) to 1/3,200× (column 1-10). Columns 11 and 12 contained no primary antibody. Streptavidin-conjugated horse radish peroxidase was used to bind biotin-conjugated lectin followed by reaction with substrate. The O.D. from row G served as background. αGal-modified human serum albumin served as a positive control, while untreated HA and human serum albumin (HSA) served as negative controls. As shown in FIG. 9, a six-fold increase in lectin staining, corresponding to the overall αGal epitope content, was observed in chemically versus enzymatically modified HA peptides (compare lane C to lanes D-F). These results illustrate that chemical modification results in the addition of greater numbers of αGal residues than biologic or enzymatic approaches. Maximum modification of αGal residues may be achieved by combining a chemical modification approach with a biological or enzymatic approach.

### Example 9

### Detection and Quantification of αGal Epitopes on MoMLV-Packaged Pseudovirions

Once modified, αGal content of antiviral vaccines can be detected and quantified using several methods, including ELISA- and Western Blot-based approaches. For example, both αGal epitope-specific chicken antibodies (NewLink Genetics Corporation) and the IB4 lectin (from Griffonia simplicifolia) can be used in ELISA and Western Blot formats for the detection and semi-quantification of αGal epitopes on antiviral vaccine components. In addition, the presence of αGal epitopes on pseudovirus-based vaccines can be detected using a human serum transduction inhibition assay (described below).

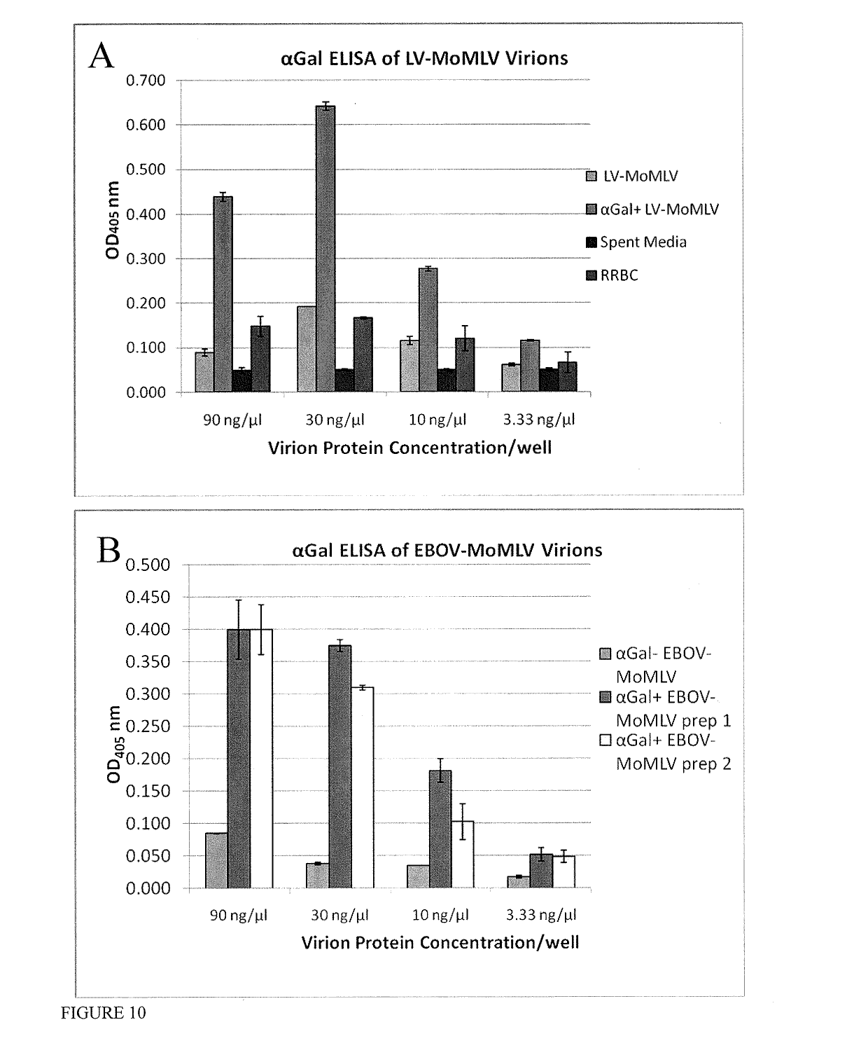

To confirm that αGal epitopes were added biologically (de novo) during the generation of virion-based antiviral vaccines from an αGal-modifying packaging cell line (Example 5), an ELISA was performed using chicken anti-αGal antibodies to probe microplate wells coated with solubilized virion proteins. Both αGal(+) and αGal(−) Ebolavirus Zaire (EBOV)- and Lassa virus (LV)-pseudotyped MoMLV were assayed. Pseudotyped MoMLV particles were solubilized with 1% Triton×100 and coated on an ELISA plate at 90, 30, 10, and 3.33 ng/l per well in duplicate. Plates were probed with an anti-αGal chicken IgY (1:500) for 1 hour and signals detected with alkaline phosphatase-conjugated rabbit anti-chicken IgY (1:1000) and DEA/pNpp substrate. Medium and rabbit red blood cells (RRBCs) were used as negative and positive control coating agents, respectively. Results of these experiments indicate that a signal was obtained with control (αGal⁺) RRBCs (maximal at 30 ng/μl), while the signal from the medium represented background (FIG. 10A). Importantly, the signal obtained for LV GP-pseudotyped MoMLV from αGal(+) cells was substantially greater than that for virions generated by αGal(−) cells, indicating that the virions from αGal(+) cells are modified with αGal epitopes. As shown in FIG. 10B, a similar result was obtained when EBOV GP-pseudotyped MoMLV were assayed: virions generated by αGal(+) packaging cells show a substantially higher signal than those from αGal(−) cells, consistent with the presence of αGal epitopes on virions generated by αGal(+) packaging cells.

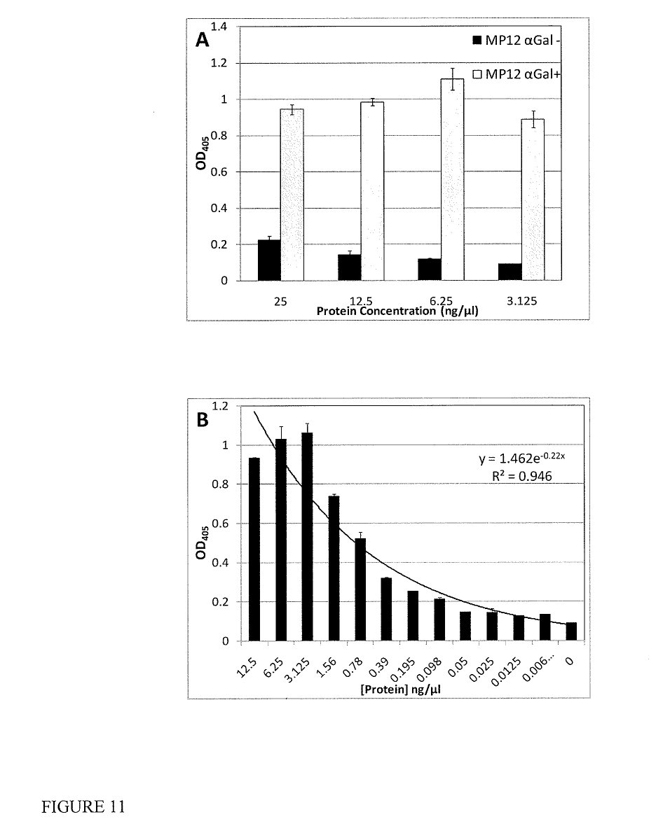

A similar experiment was conducted to detect the presence of αGal epitopes on Rift Valley Fever Virus (RVFV) pseudotyped MoMLV virions. The RVFV vaccine strain MP12 was generated in an α1,3 GT+293 cell line (described in Example 5). An ELISA scheme similar to that described above for the EBOV and LV pseudotyped MoMLV particles was employed to assay for the presence of αGal epitopes in the RVFV pseudotyped MoMLV virions. FIG. 11A shows a comparison of anti-αGal staining of solubilized virions generated in α1,3 GT+ and native (α1,3 GT−) 293 cells. While the overall amplitude of the positive signals was not substantially different (suggesting that the coating agent was possibly at saturating levels), the signal obtained for material derived from the α1,3 GT+ cell line was substantially greater than that from the native cell line, indicating that α1,3 GT+ cell lines were capable of modifying vaccine candidates with αGal epitopes.

To develop a semi-quantitative ELISA for determining relative αGal content on virions, select dilutions of solubilized αGal+MP12 were probed with anti-αGal chicken antibodies. Linear regression yielded an equation with a r² value of 0.95 (FIG. 11B), demonstrating that standards can be generated for the comparison of αGal+MP12 preparations. This general approach can be used to assess αGal levels on other types of antiviral vaccine preparations.

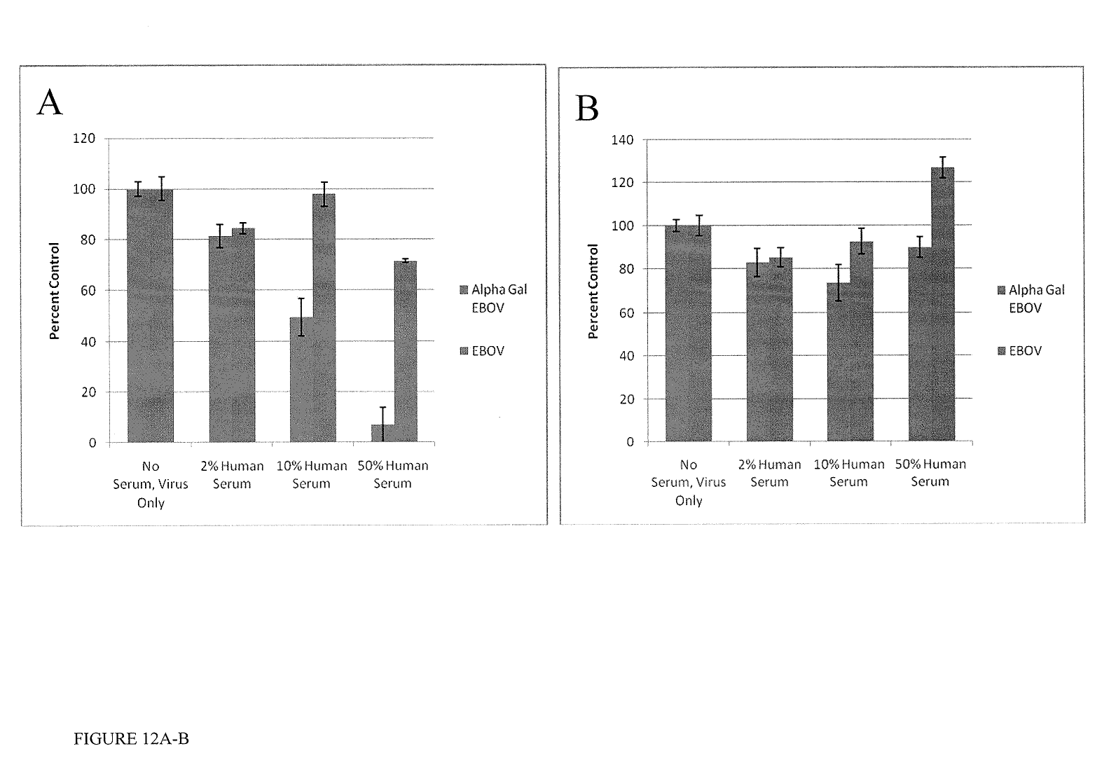

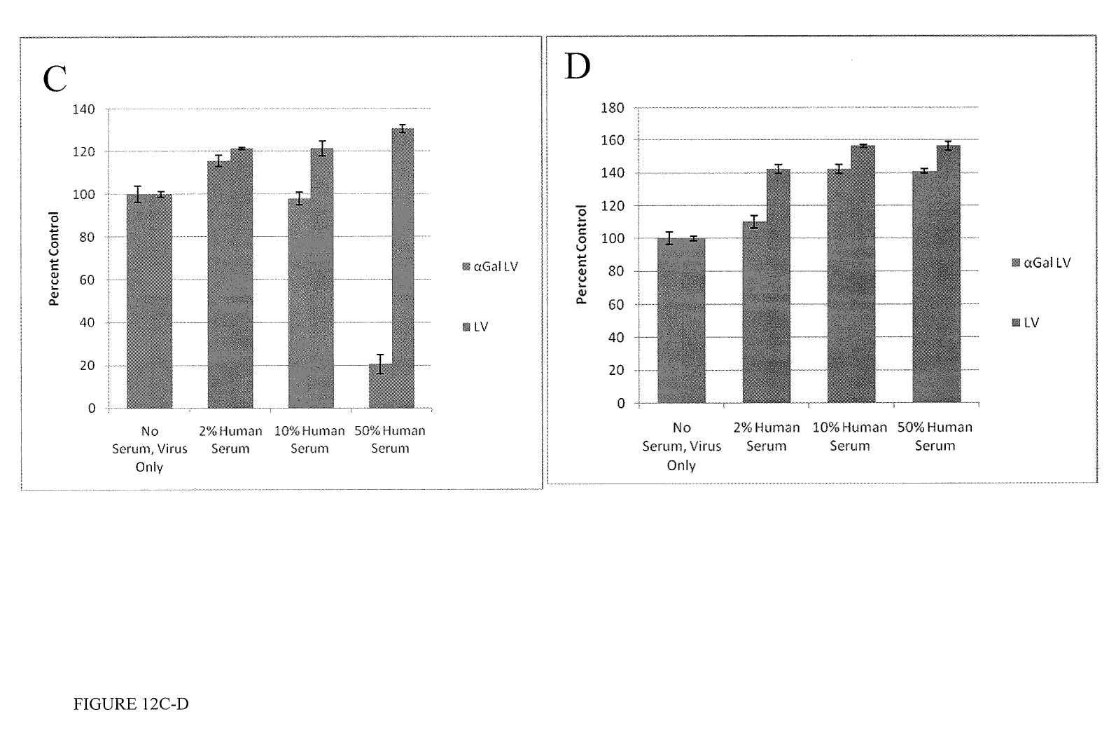

A transduction inhibition assay was employed to confirm the presence of αGal epitopes on functional pseudotyped MoMLV. This assay is based on the αGal-dependent, complement-mediated inactivation of pseudovirions by the anti-αGal antibodies found in human serum. Ebola ΔO (mucin domain-deleted) glycoprotein (GP)- and Lassa Virus GP-pseudotyped MoMLV particles containing GFP replication-defective genomes were generated in either αGal(+) or αGal(−) packaging cells. Virions were exposed to normal (FIGS. 12A and C) or heat-inactivated (FIGS. 12B and D) human sera for 60 minutes and antiviral activity was subsequently determined by transduction of Vero cells. Transduced populations were analyzed for GFP expression 72 hours post-transduction for EBOV GP-MoMLV and 120 hours post-transduction for LV GP-MoMLV. MoMLV pseudotyped with either LV GPs or EBOV Δ0 GPs produced in α1,3 GT+ packaging cell lines were rapidly and efficiently inactivated, in a complement-dependent manner, by normal human serum (FIGS. 12A and C). Transduction was not affected by fetal bovine serum (data not shown) or heat-inactivated human serum (FIGS. 12B and D). These results provide additional confirmation of the presence of Gal epitopes on pseudovirions generated in the developed packaging cell lines.

### Example 10

### Characterization of MoMLV Packaging Cell Lines

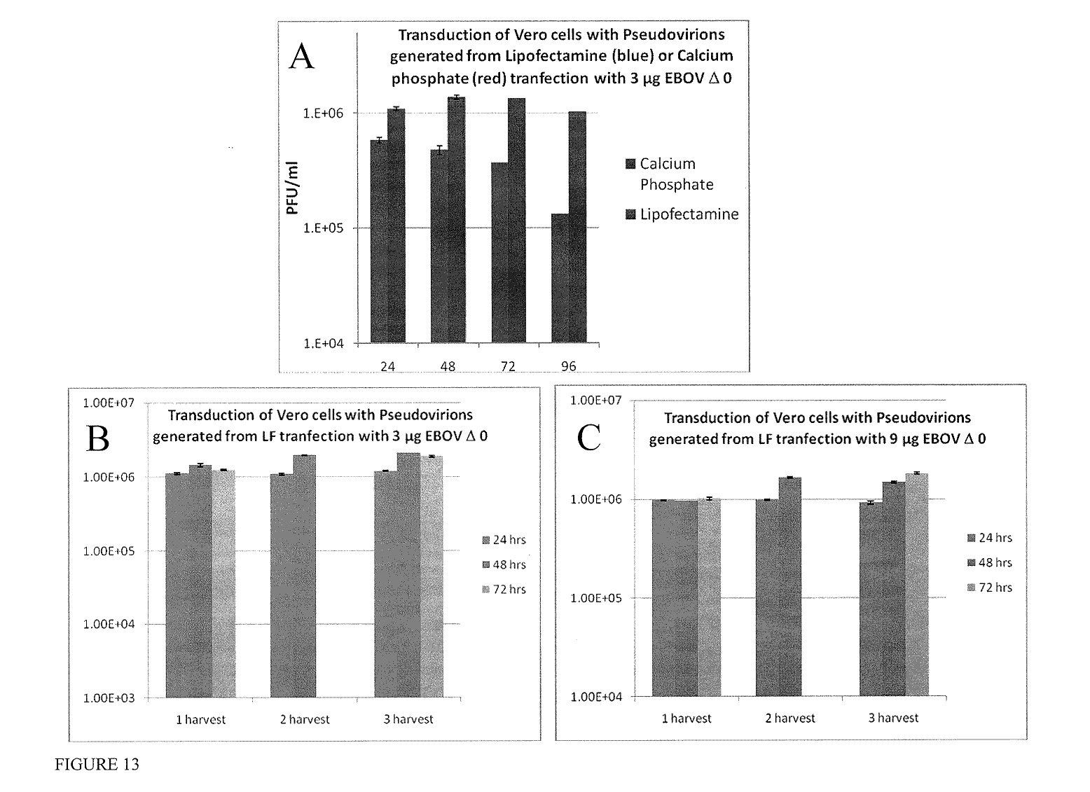

The effects of transfection technique and amount of glycoprotein expression plasmid on pseudovirus production from a MoMLV packaging cell line were examined. The use of a liposome-based (Lipofectamine 2000, Invitrogen) transfection technique resulted in a significantly higher EBOV GP-pseudotyped MoMLV titer than a chemical (calcium phosphate, Invitrogen) transfection method at all harvest points (FIG. 13A). The difference in titer approached 10-fold at 96 hours.

A direct comparison of pseudovirus titers resulting from transfection experiments using 3 or 9 μg of EBOV GP expression plasmid showed a significant increase in pseudovirus titers when 3 μg was used (FIGS. 13B and C). At 24 hours, cells transfected with 3 or 9 μg yielded 1.12×10⁶ and 9.83×10⁵ pfu/ml, respectively, and at 48 hours yielded 1.46×10⁶ and 9.71×10⁵ pfu/ml, respectively. This result clearly demonstrates that the amount of transfected glycoprotein expression plasmid has a significant effect on pseudovirion production.

To determine the nature of the particles generated from the MoMLV packaging cell lines, Western Blot analysis for the MoMLV Gag protein of the supernatant from transfected cells was performed. MoMLV Gag is visualized as a ˜50 kD species when it is expressed intracellularly or in unbudded MoMLV particles. If MoMLV pol is present in the virion particles along with gag, as is the case when virions (pseudovirus) are generated from a MoMLV gag+pol+packaging cell line, gag will be cleaved into species of ˜30, and 15 kD after the virion has budded from the cell. Thus, the change in gag staining pattern is indicative of whether it is present in pol-negative MoMLV particles (no pol) or MoMLV pseudovirions (with gag and pol).

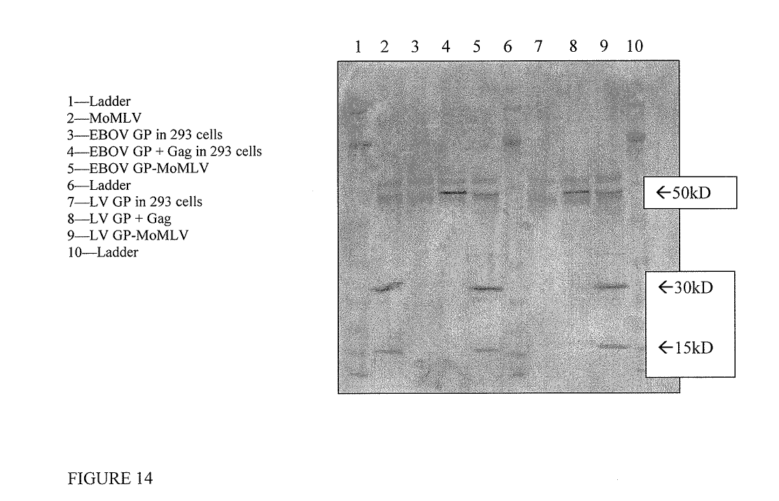

As shown in FIG. 14, lane 2 corresponding to the supernatant of the gag+pol+ packaging cell line shows bands of 30 and 15 kD, indicating the purified material is MoMLV pseudovirions (i.e. presence of pol). Lanes 3 and 7, which correspond to the supernatant from 293 cells transfected with either EBOV or LV glycoprotiens, do not contain reactive species as no gag was transfected into the cells. Lanes 4 and 8 corresponding to the supernatant from 293 cells transfected with MoMLV gag and either EBOV or LV glycoproteins show a ˜50 kD species, consistent with the purification of MoMLV particles containing gag but no pol. Lanes 5 and 9 containing supernatant from the gag+pol+ packaging cell line transfected with either EBOV or LV glycoproteins contain species of 30 and 15 kD (as well as some uncleaved ˜50 kD species), indicating the presence of pol in the purified material. The purified material is likely pseudovirions containing the EBOV and LV GPs, and both gag and pol.

The effect of harvest frequency on pseudovirion production from the MoMLV packaging cells lines was tested. The results of the experiments (shown in Table 1) show that more frequent harvests lead to the purification of substantially more (94%) pseudovirus from a single transfection experiment. The number of plaque forming units (pfu) was determined by transduction of permissive Vero cells and subsequent FACS analysis for GFP expression conveyed by the incorporated replication-defective genome.

TABLE 1
Total pfu of purified pseudovirus is dependent upon harvest frequency.

| | Single harvest | Multiple harvest | Multiple harvest |
|---|---|---|---|
| Harvest Time | method | method (24 hr) | method (12 hr) |
| [h] | (PFU/ml) | (PFU/ml) | (PFU/ml) |
| 12 | — | — | 6.24 × 10⁵ |
| 24 | — | 8.15 × 10⁵ | 1.27 × 10⁶ |
| 36 | — | — | 1.49 × 10⁶ |
| 48 | — | 1.65 × 10⁶ | 1.16 × 10⁶ |
| 60 | — | — | 1.51 × 10⁶ |
| 72 | 1.33 × 10⁶ | 1.33 × 10⁶ | 1.31 × 10⁶ |
| 84 | — | — | 1.27 × 10⁶ |
| 96 | — | 1.16 × 10⁶ | 1.2 × 10⁶ |
| 108 | — | — | 8.12 × 10⁵ |
| 120 | — | 9.41 × 10⁵ | 7.00 × 10⁵ |
| 132 | — | — | 5.65 × 10⁵ |
| 144 | — | 5.98 × 10⁵ | 5.98 × 10⁵ |
| 156 | | — | 1.28 × 10⁵ |
| 168 | — | 5.9 × 10⁴ | 2.76 × 10⁴ |

### Example 11

### Generation of Pseudovirions Containing Rift Valley Fever Virus Glycoproteins for Vaccine Preparations

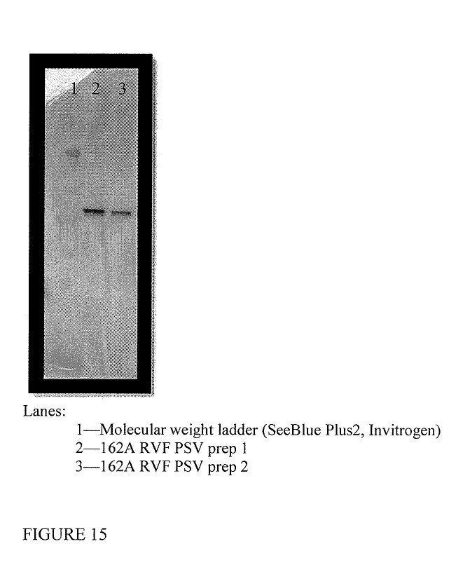

The developed MoMLV packaging cell lines were used to generate pseudovirions containing Rift Valley Fever Virus (RVFV) glycoproteins. Expression plasmids for the RVFV G_{N} and G_{C} proteins were transfected into the MoMLV packaging cell line. Supernatant was harvested 24, 48, 72, and 96 hours post transfection. Pooled supernatant from each time point was concentrated using ultracentrifugation. Western blot analysis of aliquots at each time point was performed to visualize purified pseudovirions containing RVFV glycoproteins. A reactive species with the approximate expected size of RVFV G_{N} (56 kD) was visible from each preparation (FIG. 15, lanes 2 and 3) confirming the presence of RVFV G in the purified MoMLV pseudovirions.

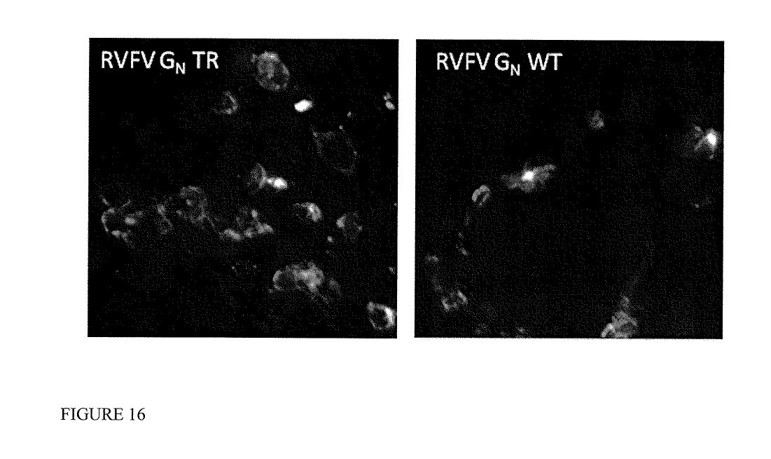

Since MoMLV pseudovirus buds from the plasma membrane, localization of RVFV glycoprotein to the cell surface should increase pseudovirus production. However, wild type (WT) RVFV glycoprotein localizes to the Golgi, and budding of WT RVFV occurs at the Golgi membrane. Golgi localization of RVFV glycoprotein is dependent upon a Golgi targeting sequence present in G_{N}. Therefore, to increase pseudovirus production, the localization of G_{N} (and thus G_{C}) was re-directed to the plasma membrane by removing the Golgi localization signal present in the cytoplasmic domain of G_{N}. The cytoplasmic domain of the G_{N} glycoprotein from RVFV was exchanged for the cytoplasmic domain from the MoMLV envelope protein (TR). The MoMLV packaging cell line was then transfected with either this chimeric RVFV G_{N} (FIG. 16, left panel) or wild type (WT) G_{N} (FIG. 16, right panel). 48 hours after transfection, cells were fixed with 2% paraformaldehyde for maintenance of cellular integrity and visualization of cell surfaces (minimum cell permeabilization). Fixed cells were then probed with an anti-RVFV G_{N} polyclonal sera and visualized with AlexaFluor 488-conjugated goat anti-rabbit antibodies (Invitrogen). As shown in FIG. 16, more surface staining is visible for cells transfected with the chimeric G_{N} glycoprotein compared to those expressing WT G_{N}. These results suggest that the Golgi retention signals were not functional in the chimeric construct.

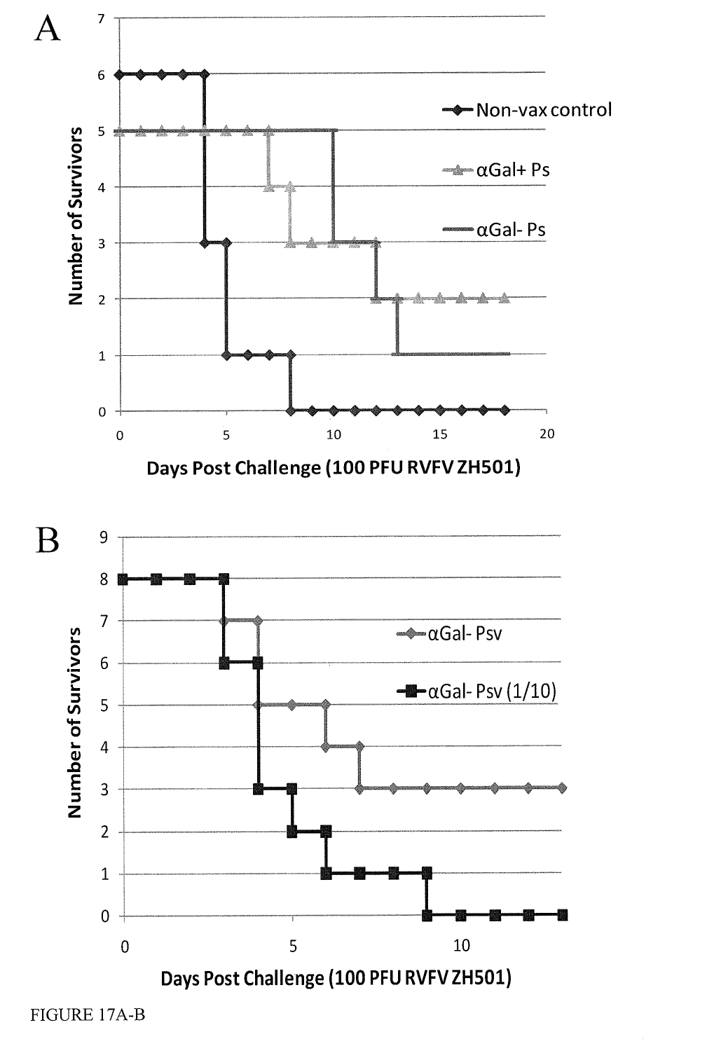

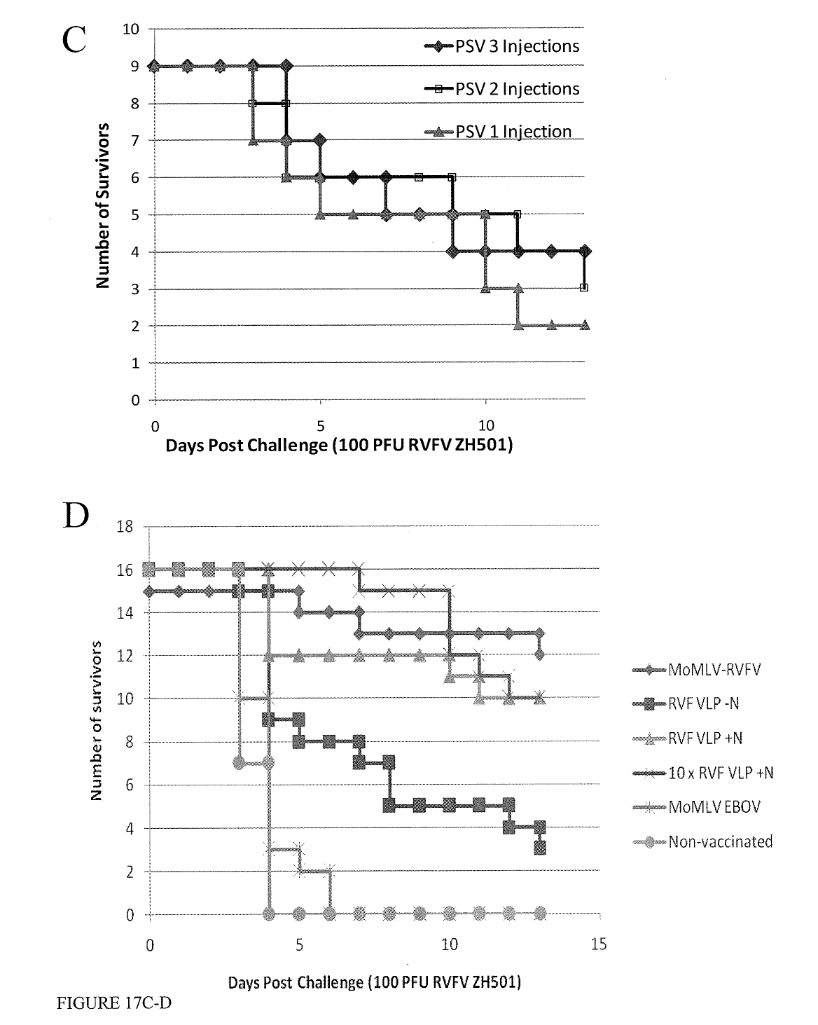

To determine if RVFV pseudovirus produced by the inventive MoMLV packaging cell line was effective as a vaccine, mice lacking α1,3 galactosyl transferase (α1,3 GT-KO) were immunized s.c. with αGal-modified or unmodified RVFV GP-pseudotyped MoMLV followed by boosters at 2-week intervals. Mice were then challenged 7 weeks post first vaccination with 100 pfu RVFV ZH501. Control mice received no vaccine or EBOV-GP-pseudotyped MoMLV. All vaccines were administered with the Sigma Adjuvant System™ (Catalog Number S6322) adjuvant per manufacturer's recommendations. The results of this series of experiments (shown in FIG. 17A) clearly demonstrate that the RVFV GP-pseudotyped MoMLV based vaccine candidate is protective in a lethal challenge model. αGal-modified pseudotyped MoMLV was significantly more protective than unmodified pseudovirus. Importantly, nonvaccinated animals, or animals vaccinated with EBOV GP-pseudotyped MoMLV with adjuvant died within 8 days post-RVFV challenge indicating that protection was dependent upon the RVFV-specific vaccine. Interestingly, a 1/10 dose reduction of αGal-modified pseudotyped MoMLV was not protective (FIG. 17B). However, a reduction in vaccination number still proved to be protective (FIG. 17C), such that protection levels were dependent upon the number of vaccinations (3>2>1). To test the efficacy of various vaccine preparations comprising RVFV pseudovirus or virus-like particles (VLPs), α1,3 GT-KO mice were immunized s.c. with αGal-modified RVFV GP-pseudotyped MoMLV or RVF VLPs followed by boosters at 2-week intervals. Mice were then challenged 7 weeks post first vaccination with 100 pfu RVFV ZH501. RVF VLPs were generated in α1,3 GT+293 cells and contained either MoMLV gag, RVFV glycoprotein, and RVFV nucleoprotein (RVF VLP+N) or MoMLV gag and RVFV glycoprotein (RVF VLP-N). Control mice received no vaccine or EBOV-GP-pseudotyped MoMLV. As shown in FIG. 17D, both RVFV pseudovirus and RVF VLPs are protective (80 and 63% survivors, respectively). Importantly, RFV VLPs containing the MoMLV gag and the RVFV GP but no RVFV nucleoprotein (N) only protected 19% of the animals, indicating the importance of the nucleoprotein for vaccine efficacy. A higher dose level (10×) of the RVF VLP+N resulted in a delay of death, but not an overall increased protection versus the normal (1×) dose.

### Example 12

### αGal-Modified Pseudovirions Containing Ebola Glycoproteins as Vaccine Candidates

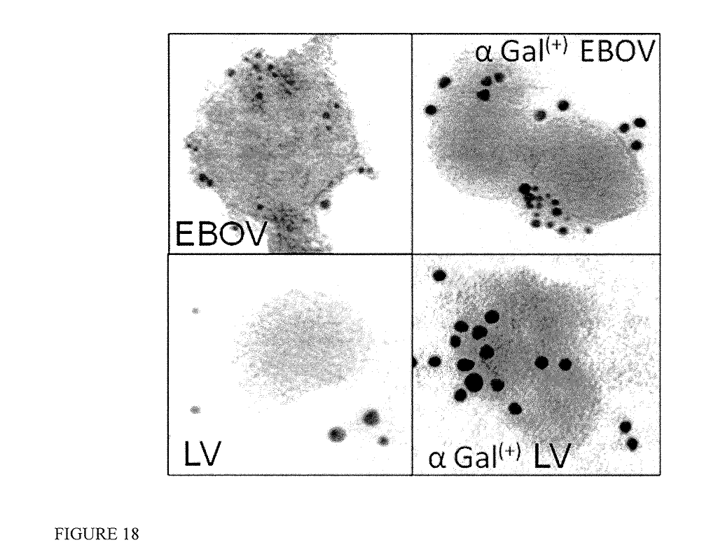

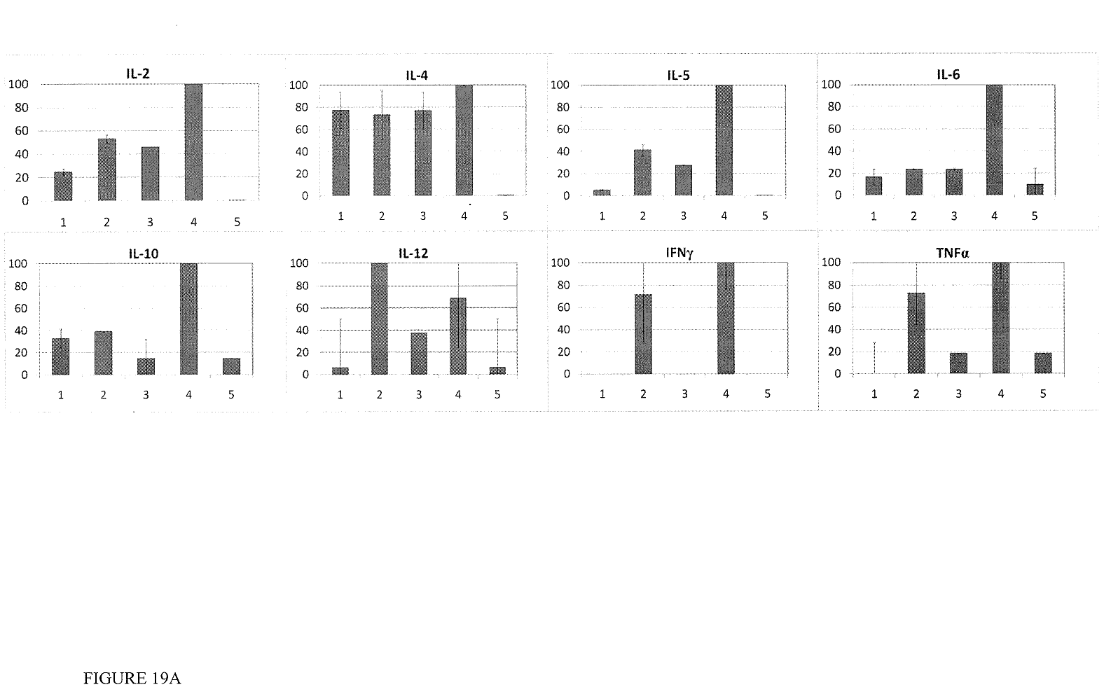

αGal+EBOV GP and LV GP-pseudotyped MoMLV generated from a MoMLV packaging cell line containing a stably integrated murine α1,3 galactosyltransferase gene (see Example 5) were probed with rabbit anti-EBOV GP and chicken anti-αGal antibodies. The primary antibodies were then visualized with anti-rabbit and anti-chicken antibodies coupled to small and large gold particles, respectively. As expected, the anti-EBOV GP antibodies recognized EBOV GP present on the surface of the EBOV GP pseudovirions (FIG. 18, upper left panel). No particles were associated with the surface of the LV GP virion (FIG. 18, lower left panel) since LV GP-MoMLV does not contain the Ebola glycoprotein. Anti-αGal antibodies recognized αGal epitopes on the surface of both EBOV GP— and LV GP-pseudotyped MoMLV (FIG. 18, upper and lower right panels). The αGal epitopes co-localize with the viral surface glycoproteins providing additional confirmation that the established packaging cell line is capable of generating αGal+EBOV GP— and LV GP-MoMLV pseudovirions.

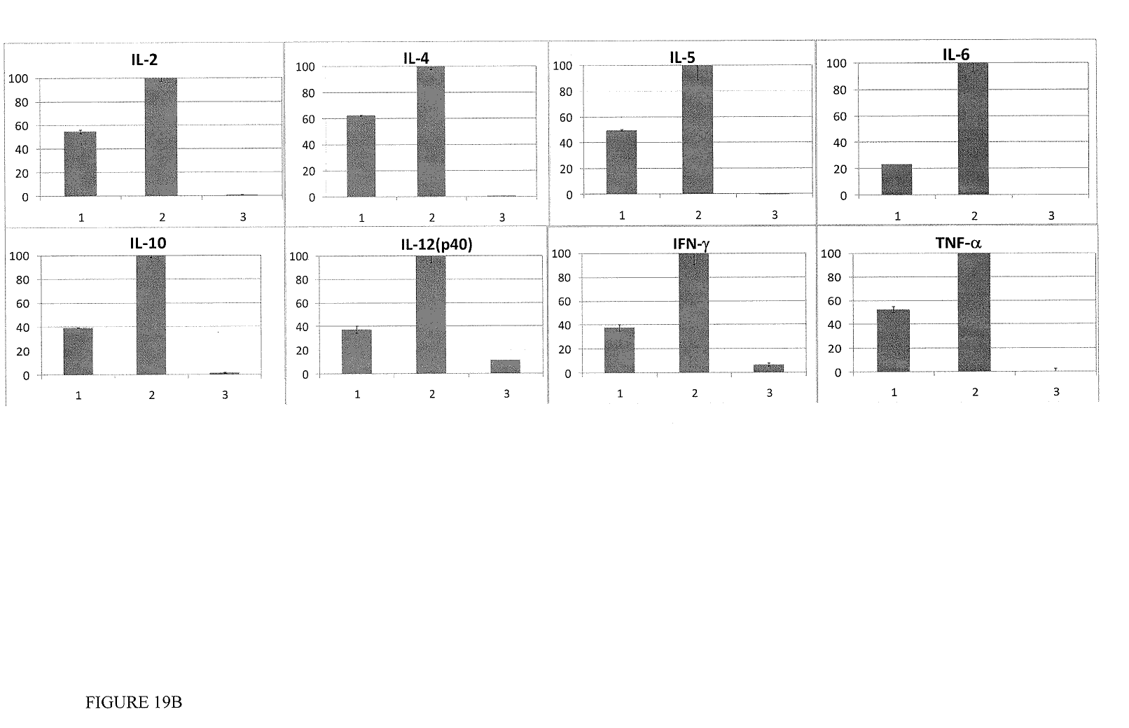

To examine whether EBOV GP-MoMLV and LV GP-MoMLV pseudovirions would be effective vaccine candidates, the immunogenicity of αGal-modified and unmodified pseudovirus was compared by measuring cytokines secreted from peripheral blood mononuclear cells (PBMCs) isolated and cultured from vaccinated α1,3 GT-KO mice. α1,3 GT-KO mice were immunized s.c. with 10⁶ or 10⁷ pfu of either αGal-modified or unmodified EBOV GP-pseudotyped MoMLV. Control mice received PBS. Six days post-injection, PBMCs from five mice were isolated by fractionation using lymphocyte separation medium, pooled, and cultured at 2.5×10⁵/well in the presence of unmodified EBOV GP-pseudotyped MoMLV for 24 hours (FIG. 19A). Another group of α1,3 GT-KO mice were immunized s.c. with 10⁷αGal-modified or unmodified LV GP-pseudotyped MoMLV, and controls received PBS. PBMCs were isolated from five mice, isolated, and cultured in the presence of unmodified LV GP-pseudotyped MoMLV for 24 hours (FIG. 19B). Secreted cytokines were then measured in culture supernatants via BioPlex analysis. As shown in FIG. 19, cytokine secretion was generally highest from PBMCs derived from mice injected with αGal-modified EBOV-pseudotyped MoMLV or αGal-modified LV-pseudotyped MoMLV as compared to their unmodified counterparts. Cytokine secretion was also higher in PBMCs isolated from mice vaccinated with 10⁷ versus 10⁶ pfu EBOV-pseudotyped MoMLV. These results are consistent with αGal-modified vaccines being more immunogenic than their unmodified counterparts. Interestingly, the cytokine profiles appear consistent with both T_{H}1 and T_{H}2 responses.

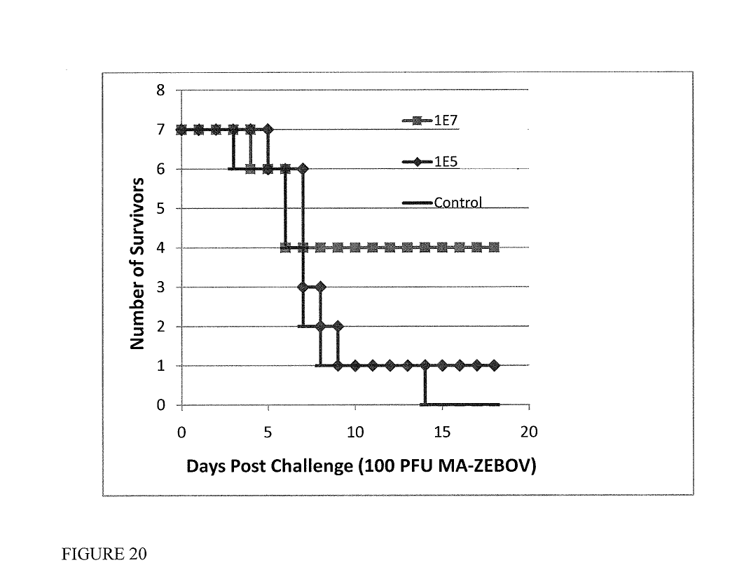

To test the efficacy of an EBOV GP-MoMLV pseudovirion-based vaccine, α1,3 GT-KO mice were immunized s.c. with 10⁷ or 10⁵ unmodified EBOV GP-pseudotyped MoMLV, followed by 2 boosters at 2-week intervals. Mice were subsequently challenged 3 weeks post final vaccination with 100 pfu mouse-adapted Zaire Ebolavirus (MA ZEBOV). Control mice received no vaccine. Vaccines were administered with the Sigma Adjuvant System™ (Catalog Number S6322) adjuvant per manufacturer's recommendations. Three injections of 10⁷ or 10⁵ pfu EBOV GP-MoMLV resulted in 57% and 29% survival, respectively (FIG. 20). Importantly, non-vaccinated animals died within 14 days post-MA-ZEBOV challenge indicating that protection was dependent upon the EBOV-specific vaccine. Interestingly, a 1/100 reduction in vaccine dose was less protective (data not shown). These results clearly demonstrate that the EBOV GP-pseudotyped MoMLV-based vaccine candidate is protective in a lethal challenge model, and protection is dose-dependent.

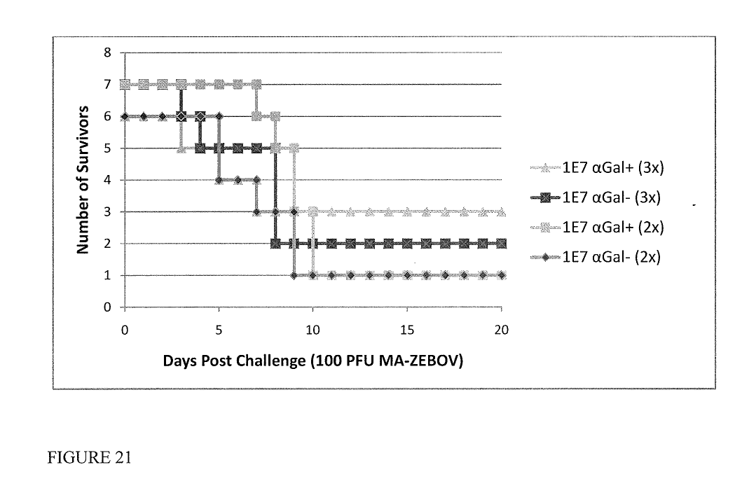

In another series of experiments, the effect of αGal modification of the pseudovirions and the number of vaccinations on survival was examined. α1,3 GT-KO mice were immunized s.c. with 10⁷ pfu αGal-modified or unmodified EBOV GP-pseudotyped MoMLV, followed by boosters at 2-week intervals (one or two). Mice were then challenged 7 weeks post first vaccination with 100 pfu mouse-adapted Ebolavirus (MA EBOV). Control mice received no vaccine. Vaccines were administered with the Sigma Adjuvant System™ (Catalog Number S6322) adjuvant per manufacturer's recommendations. As shown in FIG. 21, greater protection was obtained with αGal-modified pseudovirions (50% vs 29% survival, αGal-modified vs unmodified, respectively, three injections). Protection was also dependent on the number of vaccinations (αGal-modified: 50% vs 14% survival, three vs two injections, respectively; unmodified: 29% vs 16% survival, three vs two injections, respectively). These results show that the EBOV GP-pseudotyped MoMLV-based vaccine is protective in a lethal challenge model and the amount of protection can be increased by addition of αGal epitopes to the pseudovirions and/or increasing the number of vaccine administrations.

### Example 13

### Codon-Optimized Rift Valley Fever Virus and Lassa Virus Glycoproteins

Codon optimization of viral glycoprotein gene sequences can lead to enhanced expression of most viral glycoproteins in mammalian cells (Moore, M J et al. (2004) J. Virol., Vol. 78:10628-10635; Negrete, O A et al. (2005) Nature, Vol. 436:401-405). To increase the expression of the RVFV and Lassa virus glycoproteins in mammalian cells and increase their incorporation into pseudovirions, codon-optimized constructs were generated based on the process described in Babcock et al. (J. Virology (2004), Vol. 78:4552-4560) A codon-optimized construct (ATG4) expressing Rift Valley Fever Virus (RVFV) glycoprotein was generated. Codon optimization did not change the amino acid sequence of the translated protein as shown by the alignment below.

Clustal Alignment Verifying that the Codon-Optimized Gene Yields the Same Amino Acid Sequence as Native ATG4:

| CLUSTAL 2.0.3 multiple sequence alignment | | |
|---|---|---|
| ORIGINAL | MAGIAMTVLPALAVFALAPVVFAEDPHLRNRPGKGHNYIDGMTQEDATCKPVTYAGACSS | 60 |
| CODON_OPT | MAGIAMTVLPALAVFALAPVVFAEDPHLRNRPGKGHNYIDGMTQEDATCKPVTYAGACSS | 60 |
| | ************************************************************ | |
| ORIGINAL | FDVLLEKGKFPLFQSYAHHRTLLEAVHDTIIAKADPPSCDLLSAHGNPCMKEKLVMKTHC | 120 |
| CODON_OPT | FDVLLEKGKFPLFQSYAHHRTLLEAVHDTIIAKADPPSCDLLSAHGNPCMKEKLVMKTHC | 120 |
| | ************************************************************ | |
| ORIGINAL | PNDYQSAHHLNNDGKMASVKCPPKYELTEDCNFCRQMTGASLKKGSYPLQDLFCQSSEDD | 180 |
| CODON_OPT | PNDYQSAHHLNNDGKMASVKCPPKYELTEDCNFCRQMTGASLKKGSYPLQDLFCQSSEDD | 180 |
| | ************************************************************ | |
| ORIGINAL | GSKLKTKMKGVCEVGVQALKKCDGQLSTAHEVVPFAVFKNSKKVYLDKLDLKTEENLLPD | 240 |
| CODON_OPT | GSKLKTKMKGVCEVGVQALKKCDGQLSTAHEVVPFAVFKNSKKVYLDKLDLKTEENLLPD | 240 |
| | ************************************************************ | |
| ORIGINAL | SFVCFEHKGQYKGTMDSGQTKRELKSFDISQCPKIGGHGSKKCTGDAAFCSAYECTAQYA | 300 |
| CODON_OPT | SFVCFEHKGQYKGTMDSGQTKRELKSFDISQCPKIGGHGSKKCTGDAAFCSAYECTAQYA | 300 |
| | ************************************************************ | |
| ORIGINAL | NAYCSHANGSGIVQIQVSGVWKKPLCVGYERVVVKRELSAKPIQRVEPCTTCITKCEPHG | 360 |
| CODON_OPT | NAYCSHANGSGIVQIQVSGVWKKPLCVGYERVVVKRELSAKPIQRVEPCTTCITKCEPHG | 360 |
| | ************************************************************ | |
| ORIGINAL | LVVRSTGFKISSAVACASGVCVTGSQSPSTEITLKYPGISQSSGGDIGVHMAHDDQSVSS | 420 |
| CODON_OPT | LVVRSTGFKISSAVACASGVCVTGSQSPSTEITLKYPGISQSSGGDIGVHMAHDDQSVSS | 420 |
| | ************************************************************ | |
| ORIGINAL | KIVAHCPPQDPCLVHDCIVCAHGLINYQCHTALSAFVVVFVFSSIAIICLAILYRVLKCL | 480 |
| CODON_OPT | KIVAHCPPQDPCLVHDCIVCAHGLINYQCHTALSAFVVVFVFSSIAIICLAILYRVLKCL | 480 |
| | ************************************************************ | |
| ORIGINAL | KIAPRKVLNPLMWITAFIRWIYKKMVARVADNINOVNREIGWMEGGQLVLGNPAPIPRHA | 540 |
| CODON_OPT | KIAPRKVLNPLMWITAFIRWIYKKMVARVADNINQVNREIGWMEGGQLVLGNPAPIPRHA | 540 |
| | ************************************************************ | |
| ORIGINAL | PIPRYSTYLMLLLIVSYASACSELIQASSRITTCSTEGVNTKCRLSGTALIRAGSVGAEA | 600 |
| CODON_OPT | PIPRYSTYLMLLLIVSYASACSELIQASSRITTCSTEGVNTKCRLSGTALIRAGSVGAEA | 600 |
| | ************************************************************ | |
| ORIGINAL | CLMLKGVKEDQTKFLKLKTVSSELSCREGQSYWTGSFSPKCLSSRRCHLVGECHVNRCLS | 660 |
| CODON_OPT | CLMLKGVKEDQTKFLKLKTVSSELSCREGQSYWTGSFSPKCLSSRRCHLVGECHVNRCLS | 660 |
| | ************************************************************ | |
| ORIGINAL | WRDNETSAEFSFVGESTTMRENKCFEQCGGWGCGCFNVNPSCLFVHTYLQSVRKEALRVF | 720 |
| CODON_OPT | WRDNETSAEFSFVGESTTMRENKCFEQCGGWGCGCFNVNPSCLFVHTYLQSVRKEALRVF | 720 |
| | ************************************************************ | |
| ORIGINAL | NCIDWVHKLTLEITDFDGSVSTIDLGASSSRFTNWGSVSLSLDAEGISGSNSFSFIESPG | 780 |
| CODON_OPT | NCIDWVHKLTLEITDFDGSVSTIDLGASSSRFTNWGSVSLSLDAEGISGSNSFSFIESPG | 780 |
| | ************************************************************ | |
| ORIGINAL | KGYAIVDEPFSEIPRQGFLGEIRCNSESSVLSAHESCLRAPNLISYKPMIDQLECTTNLI | 840 |
| CODON_OPT | KGYAIVDEPFSEIPRQGFLGEIRCNSESSVLSAHESCLRAPNLISYKPMIDQLECTTNLI | 840 |
| | ************************************************************ | |
| ORIGINAL | DPFVVFERGSLPQTRNDKTFAASKGNRGVQAFSKGSVQADLTLMFDNFEVDFVGAAVSCD | 900 |
| CODON_OPT | DPFVVFERGSLPQTRNDKTFAASKGNRGVQAFSKGSVQADLTLMFDNFEVDFVGAAVSCD | 900 |
| | ************************************************************ | |
| ORIGINAL | AAFLNLTGCYSCNAGARVCLSITSTGTGSLSAHNKDGSLHIVLPSENGTKDQCQILHFTV | 960 |
| CODON_OPT | AAFLNLTGCYSCNAGARVCLSITSTGTGSLSAHNKDGSLHIVLPSENGTKDQCQILHFTV | 960 |
| | ************************************************************ | |
| ORIGINAL | PEVEEEFMYSCDGDERPLLVKGTLIAIDPFDDRREAGGESTVVNPKSGSWNFFDWFSGLM | 1020 |
| CODON_OPT | PEVEEEFMYSCDGDERPLLVKGTLIAIDPFDDRREAGGESTVVNPKSGSWNFFDWFSGLM | 1020 |
| | ************************************************************ | |
| ORIGINAL | SWFGGPLKTILLICLYVALSIGLFFLLIYLGGTGLSKMWLAATKKAS- | 1067 |
| CODON_OPT | SWFGGPLKTILLICLYVALSIGLFFLLIYLGGTGLSKMWLAATKKAS- | 1067 |
| | *********************************************** | |

The native and codon-optimized ATG4 DNA sequences are shown below in SEQ ID NOs: 1 and 2, respectively.

### Native RVFV Glycoprotein Sequence

| | |
|---|---|
| atggcagggattgcaatgacagtccttccagccttagcagtttttgctttggcacctgttgtttttgctgaagac | (SEQ ID NO: 1) |
| ccccatctcagaaacagaccagggaaggggcacaactacattgacgggatgactcaggaggatgccacatgcaaa | |
| cctgtgacatatgctggggcatgtagcagttttgatgtcttgcttgaaaagggaaaatttccccttttccagtcg | |
| tatgctcatcatagaactctactagaggcagttcacgacaccatcattgcaaaggctgatccacctagctgtgac | |
| cttctgagtgctcatgggaacccctgcatgaaagagaaactcgtgatgaagacacactgtccaaatgactaccag | |
| tcagctcatcacctcaacaatgacgggaaaatggcttcagtcaagtgccctcctaagtatgagctcactgaagac | |
| tgcaacttttgtaggcagatgacaggtgctagcctgaagaaggggtcttatcctctccaagacttgttttgtcag | |
| tcaagtgaggatgatggatcaaaattaaaaacaaaaatgaaaggggtctgcgaagtgggggttcaagcactcaaa | |
| aagtgtgatggccaactcagcactgcacatgaggttgtgccctttgcagtgtttaagaactcaaagaaggtttat | |
| cttgataagcttgaccttaagactgaggagaatctgctaccagactcatttgtctgtttcgagcataagggacag | |
| tacaaaggaacaatggactctggtcagactaagagggagctcaaaagctttgatatctctcagtgccccaagatt | |
| ggaggacatggtagtaagaagtgcactggggacgcagcattttgctctgcttatgagtgcactgctcagtacgcc | |
| aatgcctattgttcacatgctaatgggtcagggattgtgcagatacaagtatcaggggtctggaagaagccttta | |
| tgtgtagggtatgagagagtggttgtgaagagagaactctctgccaagcccatccagagagttgagccttgcaca | |
| acttgtataaccaaatgtgagcctcatggattggttgtccgatcaacagggttcaagatatcatcagcagttgct | |
| tgtgctagcggagtttgcgtcacaggatcgcagagtccttccaccgagattacactcaagtatccagggatatcc | |
| cagtcttctgggggggacataggggttcacatggcacacgatgatcagtcagttagctccaaaatagtagctcac | |
| tgccctccccaggacccgtgcttagtgcatgactgcatagtgtgtgctcatggcctgataaattaccagtgtcac | |
| actgctctcagtgcctttgttgttgtgtttgtattcagttctattgcaataatttgtttagctattctttatagg | |
| gtgcttaagtgcctgaagattgccccaaggaaagttctgaatccactaatgtggatcacagccttcatcagatgg | |
| atatataagaagatggttgccagagtggcagacaacattaatcaagtgaacagggaaataggatggatggaagga | |
| ggtcagttggttctagggaaccctgcccctattcctcgtcatgccccaatcccacgttatagcacatacctgatg | |
| ttattattgattgtctcatatgcatcagcatgttcagaactgattcaggcaagctccagaatcaccacttgctct | |
| acagagggtgttaacaccaagtgtagactgtctggcacagcattgatcagagcagggtcagttggggcagaggct | |
| tgtttgatgttgaagggggtcaaggaagatcaaaccaagttcttaaagttaaaaactgtctcaagtgagctatca | |
| tgcagggagggccagagttattggactgggtcctttagccctaaatgtttgagctcaaggagatgccaccttgtc | |
| ggggaatgccatgtgaataggtgtctgtcttggagggacaatgaaacttcagcagagttttcatttgttggggaa | |
| agcacgaccatgcgagagaataagtgttttgagcaatgtggaggatgggggtgtgggtgtttcaatgtgaaccca | |
| tcttgcttatttgtgcacacgtatctgcagtcagttagaaaagaggcccttagagtttttaactgtatcgactgg | |
| gtgcataaactcactctagagatcacagactttgatggctctgtttcaacaatagacttgggagcatcatctagc | |
| cgtttcacaaactggggttcagttagcctctcactggacgcagagggcatttcaggctcaaatagcttttctttc | |
| attgagagcccaggcaaagggtatgcaattgttgatgagccattctcagaaattcctcggcaagggttcttgggg | |
| gagatcaggtgcaattcagagtcctcagtcctgagtgctcatgaatcatgccttagggcaccaaaccttatctca | |
| tacaagcccatgatagatcaattggagtgcacaacaaatctgattgatccctttgttgtctttgagaggggttct | |
| ctgccacagacaaggaatgacaaaacctttgcagcttcaaaaggaaatagaggtgttcaagctttctctaagggc | |
| tctgtacaagctgatctaactctgatgtttgacaattttgaggtggactttgtgggagcagccgtatcttgtgat | |
| gccgccttcttaaatttgacaggttgctattcttgcaatgcaggggccagggtctgcctgtctatcacatccaca | |
| ggaactggatctctctctgcccacaataaggatgggtctctgcatatagtccttccatcagagaatggaacaaaa | |
| gaccagtgtcagatactacacttcactgtgcctgaagtagaggaggagtttatgtactcttgtgatggagatgag | |
| cggcctctgttggtgaaggggaccctgatagccattgatccatttgatgataggcgggaagcagggggggaatca | |
| acagttgtgaatccaaaatctggatcttggaatttctttgactggttttctggactcatgagttggtttggaggg | |
| cctcttaaaactatactcctcatttgcctgtatgttgcattatcaattgggctctttttcctccttatatatctt | |
| ggaggaacaggcctctctaaaatgtggcttgctgccactaagaaggcctcatag | |

Codon Optimized RVFV Glycoprotein Sequence: Changes from Native Sequence are Capitalized

| | |
|---|---|
| atggcCggCattgcTatgacagtGctGccTgccCTGgcCgtGttCgctCtggcCcctgtGgtGttCgctgaagac | (SEQ ID NO: 2) |
| ccccatctcagaaacagaccCggAaagggCcacaactacattgacggAatgacAcaggaggatgccacatgcaaa | |
| cctgtgacatatgctggCgcCtgtagcagCttCgatgtGCtgctCgaaaagggaaaattCcccctCttccagtcc | |
| tatgctcatcatagaacCctGctGgaggcTgtGcacgacaccatcattgcCaaggctgatccTcctagctgtgac | |
| cttctCagCgctcatggAaacccctgcatgaaagagaaactcgtgatgaagacacactgtccCaatgactaccag | |
| tcCgcGcatcacctcaacaatgacggCaaaatggcttcCgtGaagtgccctcctaagtatgagctcacAgaagac | |
| tgcaacttCtgtaggcagatgacaggCgctagcctgaagaagggCtcGtatcctctccaagacCtgttCtgtcag | |
| tcCagCgaggatgatggatcCaaaCtGaaaacaaaaatgaaaggCgtGtgcgaagtgggAgtGcaagcCctcaaa | |
| aagtgtgatggccaactcagcacCgcTcatgaggtGgtgcccttCgcCgtgttCaagaaCtcCaagaaggtGtat | |
| ctGgataagctGgacctCaagacCgaggagaatctgctCccTgactcCttCgtGtgtttcgagcataagggacag | |
| tacaaaggaacaatggactcCggAcagacAaagagggagctcaaaagcttCgatatctcCcagtgccccaagatt | |
| ggaggacatggAagCaagaagtgcacAggCgacgcagcattCtgctcCgcttatgagtgcacCgctcagtacgcc | |
| aatgcctattgttcCcatgctaatggCtcCggAattgtgcagatCcaagtGtcCggCgtGtggaagaagcctCtC | |
| tgtgtGggCtatgagagagtggtGgtgaagagagaactctcCgccaagcccatccagagagtGgagccttgcaca | |
| acCtgtatTaccaaatgtgagcctcatggaCtggtGgtcAgatcCacaggCttcaagatTtcCtcCgcCgtGgct | |
| tgtgctagcggagtGtgcgtGacaggatcCcagagCccttccaccgagattacactcaagtatccTggCatTtcc | |
| cagtcCtcCggCggAgacatCggCgtGcacatggcCcacgatgatcagtcCgtGagctccaaaatTgtGgctcac | |
| tgccctccccaggacccTtgcCtCgtgcatgactgcatTgtgtgtgctcatggcctgatCaattaccagtgtcac | |
| acAgctctcagCgccttCgtGgtGgtgttCgtGttcagCtcCattgcTatCatttgtCtCgctattctGtatagA | |
| gtgctGaagtgcctgaagattgccccTagAaaagtGctgaatccCctGatgtggatcacagccttcatcagatgg | |
| atTtataagaagatggtCgccagagtggcCgacaacattaatcaagtgaacagggaaatCggatggatggaagga | |
| ggCcagCtggtGctCggCaaccctgcccctattcctAgAcatgccccCatcccCAgGtatagcacatacctCatg | |
| CTGCtCCtgattgtGtcCtatgcCtcCgcTtgttcCgaactgattcaggcaagctccagaatcaccacAtgctcC | |
| acagagggCgtGaacaccaagtgtagactgtcCggcacagcCCtgatcagagcTggCtcCgtGggAgcTgaggct | |
| tgtCtgatgCtgaagggAgtGaaggaagatcaaaccaagttcCtCaagCtGaaGacAgtGtcCagCgagctCtcC | |
| tgTagggagggccagagCtattggacAggAtccttCagccctaaatgtCtgagctcCaggagatgccacctCgtG | |
| ggCgaatgccatgtgaataggtgtctgtcCtggagggacaatgaaacCtcCgcTgagttCTcCttCgtGggCgaa | |
| agcacAaccatgAgagagaataagtgtttCgagcaatgtggaggatgGggCtgtggAtgtttcaatgtgaacccT | |
| tcCtgcCtGttCgtgcacacCtatctgcagtcCgtGagaaaagaggccctCagagtGttCaactgtatcgactgg | |
| gtgcataaactcacActGgagatcacagacttCgatggctcCgtGtcCacaatCgacCtgggagcTtcCtcCagc | |
| AgAttcacaaactggggAtcCgtGagcctctcCctggacgcCgagggcatttcCggctcCaatagcttCtcCttc | |
| attgagagcccTggcaaaggAtatgcTattgtGgatgagccTttctcCgaaattcctAggcaaggAttcCtgggC | |
| gagatcaggtgcaattcCgagtcctcCgtGctgagCgctcatgaatcCtgcctGagggcTccCaacctCatctcC | |
| tacaagcccatgatTgatcaaCtggagtgcacaacaaatctgattgatcccttCgtGgtGttCgagaggggCtcC | |
| ctgccCcagacaaggaatgacaaaaccttCgcTgcttcCaaaggaaatagaggCgtGcaagctttctcCaagggc | |
| tcCgtGcaagctgatctGacActgatgttCgacaatttCgaggtggacttCgtgggagcTgccgtGtcCtgtgat | |
| gccgccttcCtCaatCtgacaggCtgTtattcCtgcaatgcaggCgccagggtGtgcctgtcCatcacatccaca | |
| ggaacAggatcCctctcCgcccacaataaggatggAtcCctgcatatCgtGctCccTtcCgagaatggaacaaaa | |
| gaccagtgtcagatTctCcacttcacAgtgcctgaagtGgaggaggagttCatgtactcCtgtgatggagatgag | |
| AggcctctgCtggtgaagggAaccctgatTgccattgatccCttCgatgataggAgggaagcTggCggAgaatcC | |
| acagtGgtgaatccCaaatcCggatcCtggaatttcttCgactggttCtcCggactcatgagCtggttCggaggC | |
| cctctCaaaacAatTctcctcatttgcctgtatgtGgcTCtCtcCattggCctcttCttcctcctGatTtatctC | |
| ggaggaacaggcctctcCaaaatgtggctCgctgccacAaagaaggcctcCtag | |

Final configuration of codon-optimized ATG4 construct, with EcoRI 5′ and XhoI 3′ restriction endonuclease sites (GAATTC and CTCGAG, respectively) and 5′ Kozak sequence (GCCACC, underlined), is shown below as SEQ ID NO: 3.

| | |
|---|---|
| AATATAGAATTCGCCACCatggcCggCattgcTatgacagtGctGccTgccCTGgcCgtGttCgctCtggcCcct | (SEQ ID NO: 3) |
| gtGgtGttCgctgaagacccccatctcagaaacagaccCggAaagggCcacaactacattgacggAatgacAcag | |
| gaggatgccacatgcaaacctgtgacatatgctggCgcCtgtagcagCttCgatgtGCtgctCgaaaagggaaaa | |
| ttCcccctCttccagtcctatgctcatcatagaacCctGctGgaggcTgtGcacgacaccatcattgcCaaggct | |
| gatccTcctagctgtgaccttctCagCgctcatggAaacccctgcatgaaagagaaactcgtgatgaagacacac | |
| tgtccCaatgactaccagtcCgcGcatcacctcaacaatgacggCaaaatggcttcCgtGaagtgccctcctaag | |
| tatgagctcacAgaagactgcaacttCtgtaggcagatgacaggCgctagcctgaagaagggCtcGta | |
| tcctctccaagacCtgttCtgtcagtcCagCgaggatgatggatcCaaaCtGaaaacaaaaatgaaaggCgtGtg | |
| cgaagtgggAgtGcaagcCctcaaaaagtgtgatggccaactcagcacCgcTcatgaggtGgtgcccttCgcCgt | |
| gttCaagaaCtcCaagaaggtGtatctGgataagctGgacctCaagacCgaggagaatctgctCccTgactcCtt | |
| CgtGtgtttcgagcataagggacagtacaaaggaacaatggactcCggAcagacAaagagggagctcaaaagctt | |
| CgatatctcCcagtgccccaagattggaggacatggAagCaagaagtgcacAggCgacgcagcattCtgctcCgc | |
| ttatgagtgcacCgctcagtacgccaatgcctattgttcCcatgctaatggCtcCggAattgtgcagatCcaagt | |
| GtcCggCgtGtggaagaagcctCtCtgtgtGggCtatgagagagtggtGgtgaagagagaactctcCgccaagcc | |
| catccagagagtGgagccttgcacaacCtgtatTaccaaatgtgagcctcatggaCtggtGgtcAgatcCacagg | |
| CttcaagatTtcCtcCgcCgtGgcttgtgctagcggagtCtgcgtGacaggatcCcagagCccttccaccgagat | |
| tacactcaagtatccTggCatTtcccagtcCtcCggCggAgacatCggCgtGcacatggcCcacgatgatcagtc | |
| CgtGagctccaaaatTgtGgctcactgccctccccaggacccTtgcCtCgtgcatgactgcatTgtgtgtgctca | |
| tggcctgatCaattaccagtgtcacacAgctctcagCgccttCgtGgtGgtgttCgtGttcagCtcCattgcTat | |
| CatttgtCtCgctattctGtatagAgtgctGaagtgcctgaagattgccccTagAaaagtGctgaatccCctGat | |
| gtggatcacagccttcatcagatggatTtataagaagatggtCgccagagtggcCgacaacattaatcaagtgaa | |
| cagggaaatCggatggatggaaggaggCcagCtggtGctCggCaaccctgcccctattcctAgAcatgccccCat | |
| cccCAgGtatagcacatacctCatgCTGCtCCtgattgtGtcCtatgcCtcCgcTtgttcCgaactgattcaggc | |
| aagctccagaatcaccacAtgctcCacagagggCgtGaacaccaagtgtagactgtcCggcacagcCCtgatcag | |
| agcTggCtcCgtGggAgcTgaggcttgtCtgatgCtgaagggAgtGaaggaagatcaaaccaagttcCtCaagCt | |
| GaaGacAgtGtcCagCgagctCtcCtgTagggagggccagagCtattggacAggAtccttCagccctaaatgtCt | |
| gagctcCaggagatgccacctCgtGggCgaatgccatgtgaataggtgtctgtcCtggagggacaatgaaacCtc | |
| CgcTgagttCTcCttCgtGggCgaaagcacAaccatgAgagagaataagtgtttCgagcaatgtggaggatgGgg | |
| CtgtggAtgtttcaatgtgaacccTtcCtgcCtGttCgtgcacacCtatctgcagtcCgtGagaaaagaggccct | |
| CagagtGttCaactgtatcgactgggtgcataaactcacActGgagatcacagacttCgatggctcCgtGtcCac | |
| aatCgacCtgggagcTtcCtcCagcAgAttcacaaactggggAtcCgtGagcctctcCctggacgcCgagggcat | |
| ttcCggctcCaatagcttCtcCttcattgagagcccTggcaaaggAtatgcTattgtGgatgagccTttctcCga | |
| aattcctAggcaaggAttcCtgggCgagatcaggtgcaattcCgagtcctcCgtGctgagCgctcatgaatcCtg | |
| cctGagggcTccCaacctCatctcCtacaagcccatgatTgatcaaCtggagtgcacaacaaatctgattgatcc | |
| cttCgtGgtGttCgagaggggCtcCctgccCcagacaaggaatgacaaaaccttCgcTgcttcCaaaggaaatag | |
| aggCgtGcaagctttctcCaagggctcCgtGcaagctgatctGacActgatgttCgacaatttCgaggtggactt | |
| CgtgggagcTgccgtGtcCtgtgatgccgccttcCtCaatCtgacaggCtgTtattcCtgcaatgcaggCgccag | |
| ggtGtgcctgtcCatcacatccacaggaacAggatcCctctcCgcccacaataaggatggAtcCctgcatatCgt | |
| GctCccTtcCgagaatggaacaaaagaccagtgtcagatTctCcacttcacAgtgcctgaagtGgaggaggagtt | |
| CatgtactcCtgtgatggagatgagAggcctctgCtggtgaagggAaccctgatTgccattgatccCttCgatga | |
| taggAgggaagcTggCggAgaatcCacagtGgtgaatccCaaatcCggatcCtggaatttcttCgactggttCtc | |
| CggactcatgagCtggttCggaggCcctctCaaaacAatTctcctcatttgcctgtatgtGgcTCtCtcCattgg | |
| CctcttCttcctcctGatTtatctCggaggaacaggcctctcCaaaatgtggctCgctgccacAaagaaggcctc | |
| CtagCTCGAG | |

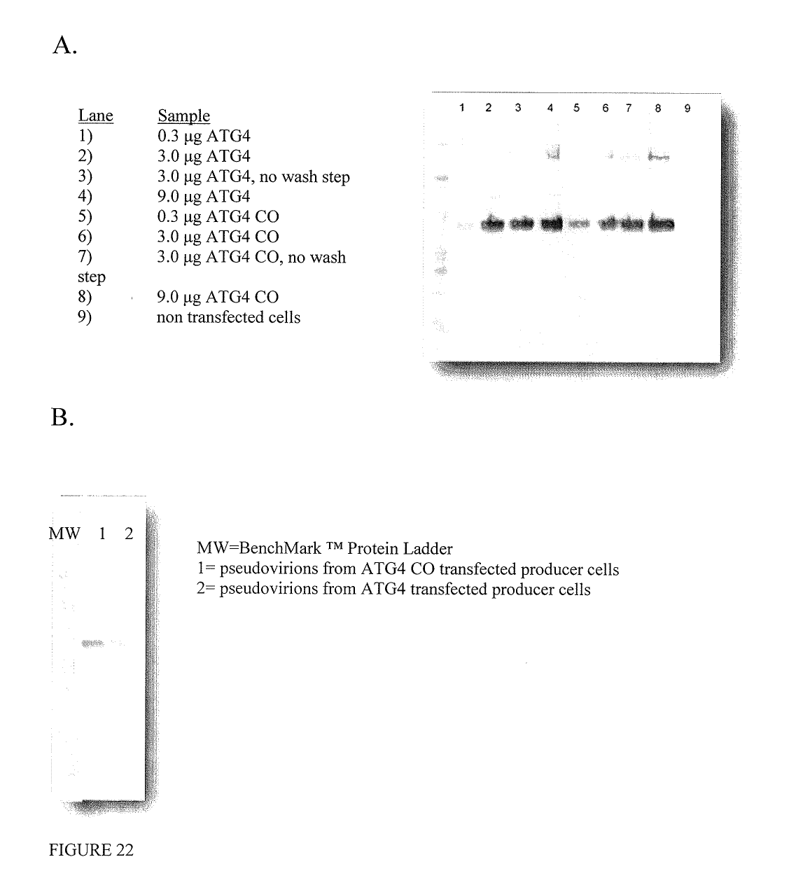

293 cells were transfected with select quantities of similar expression plasmids containing either native or codon-optimized (CO) ATG4. Cell lysates were harvested 24 hours post-transfection. 8.0 μg of each lysate was separated by SDS-PAGE and transferred to a PVDF membrane for Western analysis. Western blots were probed with an anti-RVFV G_{N} monoclonal Ab and visualized with AP-conjugated goat-anti-mouse antibodies (FIG. 22A). Western blot analysis shows that the codon-optimized RVFV glycoprotein is expressed in mammalian cells. The codon-optimized RVFV glycoprotein may be expressed in any of the MoMLV packaging cell lines to generate pseudovirions containing RVFV glycoprotein, such as those described in Example 11. These pseudovirions may be used to induce protective immunity to RVFV infection in a subject.

150 mm plates of 70% confluent BPSC-5 (sister clone of BPSC-1) cells were transfected (lipofectamine method) with 15 and 19 μg ATG4 CO (codon-optimized RVFV glycoprotein) and ATG4 (native RVFV glycoprotein), respectively. Supernatants were collected every 24 hours up to 72 and 120 hours post-transfection for ATG4 CO and ATG4, respectively. Pseudovirus was purified from 180 ml of each transfection by centrifugation (2 hours, 77000×g) through a 20% sucrose cushion and resuspended in 2.0 ml sterile 0.9% NaCl. 5.0 μl of each sample was separated by SDS-PAGE and transferred to a PVDF membrane. RVFV G_{N} was visualized with a specific MAb followed by an alkaline phosphatase conjugated goat-anti-mouse IgG and Pierce One Step NBT/BCIP developer (FIG. 22B). Interestingly, the pellet from the ATG4 CO-transfected cells was substantially larger than that from those transfected with ATG4 (FIG. 22B), suggesting that ATG4 CO leads to the generation of more pseudovirions per transfection (data not shown).

A similar construct encoding a codon-optimized glycoprotein for Lassa virus was produced. The native and codon-optimized nucleotide sequences for Lassa virus are shown below in SEQ ID NOs: 4 and 5, respectively. Codon optimization did not change the amino acid sequence of the translated protein.

### Native Lassa Virus Glycoprotein Sequence

| | |
|---|---|
| ATGGGACAAATAGTGACATTCTTCCAGGAAGTGCCTCATGTAATAGAAGAGGTGATGAACATTGTTCTCATTGCA | (SEQ ID NO: 4) |
| CTGTCTGTACTAGCAGTGCTGAAAGGTCTGTACAATTTTGCAACGTGTGGCCTTGTTGGTTTGGTCACTTTCCTC | |
| CTGTTGTGTGGTAGGTCTTGCACAACCAGTCTTTATAAAGGGGTTTATGAGCTTCAGACTCTGGAACTAAACATG | |
| GAGACACTCAATATGACCATGCCTCTCTCCTGCACAAAGAACAACAGTCATCATTATATAATGGTGGGCAATGAG | |
| ACAGGACTAGAACTGACCTTGACCAACACGAGCATTATTAATCACAAATTTTGCAATCTGTCTGATGCCCACAAA | |
| AAGAACCTCTATGACCACGCTCTTATGAGCATAATCTCAACTTTCCACTTGTCCATCCCCAACTTCAATCAGTAT | |
| GAGGCAATGAGCTGCGATTTTAATGGGGGAAAGATTAGTGTGCAGTACAACCTGAGTCACAGCTATGCTGGGGAT | |
| GCAGCCAACCATTGTGGTACTGTTGCAAATGGTGTGTTACAGACTTTTATGAGGATGGCTTGGGGTGGGAGCTAC | |
| ATTGCTCTTGACTCAGGCCGTGGCAACTGGGACTGTATTATGACTAGTTATCAATATCTGATAATCCAAAATACA | |
| ACCTGGGAAGATCACTGCCAATTCTCGAGACCATCTCCCATCGGTTATCTCGGGCTCCTCTCACAAAGGACTAGA | |
| GATATTTATATTAGTAGAAGATTGCTAGGCACATTCACATGGACACTGTCAGATTCTGAAGGTAAAGACACACCA | |
| GGGGGATATTGTCTGACCAGGTGGATGCTAATTGAGGCTGAACTAAAATGCTTCGGGAACACAGCTGTGGCAAAA | |
| TGTAATGAGAAGCATGATGAGGAATTTTGTGACATGCTGAGGCTGTTTGACTTCAACAAACAAGCCATTCAAAGG | |
| TTGAAAGCTGAAGCACAAATGAGCATTCAGTTGATCAACAAAGCAGTAAATGCTTTGATAAATGACCAACTTATA | |
| ATGAAGAACCATCTACGGGACATCATGGGAATTCCATACTGTAATTACAGCAAGTATTGGTACCTCAACCACACA | |
| ACTACTGGGAGAACATCACTGCCCAAATGTTGGCTTGTATCAAATGGTTCATACTTGAACGAGACCCACTTTTCT | |
| GATGATATTGAACAACAAGCTGACAATATGATCACTGAGATGTTACAGAAGGAGTATATGGAGAGGCAGGGGAAG | |
| ACACCATTGGGTCTAGTTGACCTCTTTGTGTTCAGTACAAGTTTCTATCTTATTAGCATCTTCCTTCACCTAGTC | |
| AAAATACCAACTCATAGGCATATTGTAGGCAAGTCGTGTCCCAAACCTCACAGATTGAATCATATGGGCATTTGT | |
| TCCTGTGGACTCTACAAACAGCCTGGTGTGCCTGTGAAATGGAAGAGATGA | |

Codon Optimized Lassa Virus Glycoprotein Sequence: Changes from Native Sequence are Colored (1st Position Changes are in Reds and 2nd or 3rd Positions Changes are in Green)

| | |
|---|---|
| ATGGGTCAGATTGTGACATTCTTCCAGGAAGTGCCTCATCTGATTGAAGAGGTGATGAACATTGTGCTCATTGCC | (SEQ ID NO: 5) |
| CTGTCCGTGCTCGCCCTGCTGAAAGGTCTGTACAATTTTGCCACCTGTGGCCTGGTGGGTCTCGTGACATTCCTC | |
| CTGCTGTGTGGTAGGTCCTGCACAACCAGCCTCTACAAAGGTGTGTACGAGCTCCAGACACTGGAACTGAACATG | |
| GAGACACTCAATATGACCATGCCTCTCTCCTGCACAAAGAACAACAGCCATCATTACATCATGGTGGGCAATGAG | |
| ACAGGCCTGGAACTGACCCTGACCAACACAAGCATTATTAATCACAAATTTTGCAATCTGTCCCATGCCCACAAA | |
| AAGAACCTCTACGACCACGCCCTCATGAGCATTATCTCCACCTTCCACCTGTCCATCCCCAACTTCAATCAGTAC | |
| GAGGCCATGAGCTGCGATTTTAATGGCGGTAACATTAGCGTGCAGTACAACCTGAGCCACAGCTACGCCGGCGAT | |
| GCCGCCAACCATTGTGGTACCGTGGCCAATGGTGTGCTCCAGACCTTTATGAGGATGGCCTGGGGTCCTAGCTAC | |
| ATTGCCCTCGACTCCGGCAGGGGCAACTGGGACTGTATTATGACCAGCTACCACTACCTGATTATCCAGAATACA | |
| ACCTGGGAAGATCACTGCCAGTTCTCCAGACCATCCCCCATCGCTTACCTCGGTCTCCTCTCCCACAGGACCAGA | |
| GATATTTACATTAGCAGAAGACTGCTCGGCACATTCACATGGACACTGTCCGATTCCGAAGGTAAAGACACACCA | |
| GGTGGCTACTGTCTGACCAGGTGGATGCTCATTGAGGCCGAACTCAAATGCTTCGGTAACACAGCCGTGGCCAAA | |
| TGTAATGAGAAGCATCATGAGGAATTTTGTGACATGCTGAGGCTGTTTGACTTCAACAAACAGGCCATTCAGAGG | |
| CTGAAAGCCGAAGCCCAGATGAGCATTCAGCTGATCAACAAAGCCGTGAACCCCCTGATTAATGACCAGCTCATC | |
| ATGAAGAACCATCTGAGGGACATCATGGGCATTCCATACTGTAATTACAGCAAGTACTGGTACCTCAACCACACA | |
| ACCACAGGTAGAACATCCCTGCCCAAATGTTGGCTCGTCTCCAATGGTTCCTACCTGAACGAGACCCACTTTTCC | |
| GATGATATTGAACAGCACCCCGACAATATGATCACCGAGATCCTCCAGAAGGAGTACATGGAGACCCAGGGTAAG | |
| ACACCACTGGGTCTGGTGGACCTCTTTGTGTTCAGCACAAGCTTCTACCTCATTAGCATCTTCCTCCACCTGGTG | |
| AAAATTCCAACACATAGGCATATTGTGGGCAAGTCCTCTCCCAAACCTCACAGACTGAATCATATGGGCATTTGT | |
| TCCTGTGGTCTCTACAAACAGCCTGGTGTGCCTGTGAAATGGAAGAGATGA | |

All publications and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A cell line comprising:
i) a stably integrated MoMLV gag;
ii) a stably integrated MoMLV pol; and
iii) at least one heterologous glycoprotein gene from an enveloped virus, wherein said enveloped virus is a high risk pathogen.

2. The cell line of claim 1, wherein the cell line does not comprise a viral replicon and wherein said cell line produces replicon-deficient viral particles.

3. The cell line of claim 2, wherein said cell line generates a titer of replicon-deficient viral particles of at least about 1.0×10⁵ cfu/ml, about 5.0×10⁵ cfu/ml, about 7.0×10⁵ cfu/ml, about 9.0×10⁵ Cfu/ml or about 1.0×10⁶ cfu/ml.

4. The cell line of claim 1, wherein said high risk pathogen is an arenavirus.

5. The cell line of claim 4, wherein said arenavirus is Lassa virus.

6. The cell line of claim 1, wherein said high risk pathogen is a filovirus.

7. The cell line of claim 6, wherein said filovirus is an Ebola virus.

8. The cell line of claim 6, wherein said filovirus is a Marburg virus.

9. The cell line of claim 1, wherein said high risk pathogen is a bunyavirus.

10. The cell line of claim 9, wherein said bunyavirus is selected from the group of viruses consisting of Hantavirus, Nairovirus, Orthobunyavirus, Phlebovirus, and Tospovirus.

11. The cell line of claim 9, wherein said bunyavirus is selected from the group of viruses consisting of Crimean Congo hemorrhagic fever virus, Rift Valley fever virus, La Crosse virus, Dugbe Virus, Hantaan Virus and Andes virus.

12. The cell line of claim 1, wherein said glycoprotein gene is stably integrated into the cell line.

13. The cell line of claim 1, wherein said glycoprotein gene is not stably integrated into the cell line.

14. The cell line of claim 1, wherein said glycoprotein gene is expressed from an inducible promoter.

15. The cell line of claim 1, wherein said cell line comprises glycoprotein genes from different high risk pathogens.

16. The cell line of claim 1, wherein said glycoprotein gene is a chimeric glycoprotein gene.

17. The cell line of claim 1, wherein said glycoprotein gene is codon-optimized for expression in mammalian cells.

18. The cell line of claim 17, wherein said glycoprotein gene comprises the sequence of SEQ ID NO: 2.

19. The cell line of claim 17, wherein said glycoprotein gene comprises the sequence of SEQ ID NO: 5.

20. The cell line of claim 1, wherein said cell line further comprises a nucleoprotein gene from a high risk pathogen.

21. The cell line of claim 1, wherein said cell line further comprises an α(1,3) galactosyltransferase gene.

22. The cell line of claim 21, wherein said α(1,3) galactosyltransferase gene is a mouse α(1,3) galactosyltransferase gene.

23. The cell line of claim 21, wherein said α(1,3) galactosyltransferase gene is stably integrated into the cell line.

24. A cell line comprising:
i) a stably integrated MoMLV gag and
ii) at least one heterologous glycoprotein gene from an enveloped virus, wherein said enveloped virus is a high risk pathogen.

25. The cell line of claim 24, wherein said cell line does not comprise a viral replicon and wherein said cell line produces replicon-deficient viral particles.

26. The cell line of claim 24, wherein said cell line further comprises an α(1,3) galactosyltransferase gene.

27. The cell line of claim 26, wherein said α(1,3) galactosyltransferase gene is stably integrated into the cell line.

28. The cell line of claim 26, wherein said α(1,3) galactosyltransferase gene is a mouse α(1,3) galactosyltransferase gene.

29. The cell line of claim 24, wherein said cell line does not comprise a pol gene.

30. The cell line of claim 25, wherein said cell line generates a titer of replicon-deficient viral particles of at least about 1.0×10⁵ cfu/ml, about 5.0×10⁵ cfu/ml, about 7.0×10⁵ cfu/ml, about 9.0×10⁵ cfu/ml or about 1.0×10⁶ cfu/ml.

31. The cell line of claim 24, wherein said high risk pathogen is an arenavirus.

32. The cell line of claim 31, wherein said arenavirus is Lassa virus.

33. The cell line of claim 24, wherein said high risk pathogen is a filovirus.

34. The cell line of claim 33, wherein said filovirus is an Ebola virus.

35. The cell line of claim 33, wherein said filovirus is a Marburg virus.

36. The cell line of claim 24, wherein said high risk pathogen is a bunyavirus.

37. The cell line of claim 36, wherein said bunyavirus is selected from the group of viruses consisting of Hantavirus, Nairovirus, Orthobunyavirus, Phlebovirus, and Tospovirus.

38. The cell line of claim 36, wherein said bunyavirus is selected from the group of viruses consisting of Crimean Congo hemorrhagic fever virus, Rift Valley fever virus, La Crosse virus, Dugbe Virus, Hantaan Virus and Andes virus.

39. The cell line of claim 24, wherein said glycoprotein gene from said high risk pathogen is stably integrated into the cell line.

40. The cell line of claim 24, wherein said glycoprotein gene from said high risk pathogen is not stably integrated into the cell line.

41. The cell line of claim 24, wherein said glycoprotein gene from said high risk pathogen is expressed from an inducible promoter.

42. The cell line of claim 24, wherein said cell line comprises glycoprotein genes from different high risk pathogens.

43. The cell line of claim 24, wherein said glycoprotein gene is a chimeric glycoprotein gene.

44. The cell line of claim 24, wherein said glycoprotein gene is codon-optimized for expression in mammalian cells.

45. The cell line of claim 44, wherein said glycoprotein gene comprises the sequence of SEQ ID NO: 2.

46. The cell line of claim 44, wherein said glycoprotein gene comprises the sequence of SEQ ID NO: 5.

47. The cell line of claim 24, wherein said cell line further comprises a nucleoprotein gene from a high risk pathogen.

48. A vaccine preparation against a high risk pathogen comprising replicon-deficient viral particles produced by the cell line of claim 2 or 25, wherein said replicon-deficient viral particles contain at least one glycoprotein from the high risk pathogen.

49. The vaccine of claim 48, wherein said high risk pathogen is an arenavirus.

50. The vaccine of claim 49, wherein said arenavirus is Lassa virus.

51. The vaccine of claim 48, wherein said high risk pathogen is a filovirus.

52. The vaccine of claim 51, wherein said filovirus is an Ebola virus.

53. The vaccine of claim 51, wherein said filovirus is a Marburg virus.

54. The vaccine of claim 48, wherein said high risk pathogen is a bunyavirus.

55. The vaccine of claim 54, wherein said bunyavirus is selected from the group of viruses consisting of Hantavirus, Nairovirus, Orthobunyavirus, Phlebovirus, and Tospovirus.

56. The vaccine of claim 54, wherein said bunyavirus is selected from the group of viruses consisting of Crimean Congo hemorrhagic fever virus, Rift Valley fever virus, La Crosse virus, Dugbe Virus, Hantaan Virus and Andes virus.

57. The vaccine of claim 48, wherein said glycoprotein comprises αGal epitopes.

58. The vaccine of claim 48, wherein said glycoprotein is a chimeric glycoprotein.

59. The vaccine of claim 48, wherein said glycoprotein is encoded by a gene that is codon-optimized for expression in mammalian cells.

60. The vaccine of claim 59, wherein said gene comprises the sequence of SEQ ID NO: 2.

61. The vaccine of claim 59, wherein said gene comprises the sequence of SEQ ID NO: 5.

62. The vaccine of claim 48, wherein said cell line further comprises a nucleoprotein gene from a high risk pathogen.

63. The vaccine of claim 48 further comprising an adjuvant.

64. A method of preparing a vaccine against a high risk pathogen comprising the steps of:
i) growing the cell line of claims 2 or 25 under conditions which allow formation of replicon-deficient viral particles;
ii) collecting and concentrating the replicon-deficient particles; and
iii) resuspending said replicon-deficient particles in a pharmaceutically acceptable buffer.

65. The method of claim 64, wherein the method further comprises chemically or enzymatically treating said replicon-deficient particles to add αGal epitopes.

66. The method of claim 64, wherein the cell line has been transfected with at least one Lassa virus gene encoding a glycoprotein.

67. The method of claim 64, wherein the cell line has been transfected with at least one Ebola virus gene encoding a glycoprotein.

68. The method of claim 64, wherein the cell line has been transfected with at least one Marburg virus gene encoding a glycoprotein.

69. The method of claim 64, wherein the cell line has been transfected with at least one bunyavirus gene encoding a glycoprotein.

70. The method of claim 64, wherein the cell line has been transfected with at least one Rift Valley fever virus gene encoding a glycoprotein.

71. The method of claim 70, wherein the glycoprotein is a chimeric glycoprotein.

72. The method of claim 64, wherein the cell line has been transfected with at least one Crimean Congo hemorrhagic fever virus gene encoding a glycoprotein.

73. The method of claim 64, wherein the cell line has been transfected with at least one high risk pathogen gene encoding a glycoprotein, wherein said gene is codon-optimized for expression in mammalian cells.

74. The method of claim 73, wherein said gene comprises the sequence of SEQ ID NO: 2.

75. The method of claim 73, wherein said gene comprises the sequence of SEQ ID NO: 5.

76. The method of claim 64, wherein the cell line has been transfected with at least one high risk pathogen gene encoding a nucleoprotein.

77. An isolated polynucleotide comprising a sequence of SEQ ID NO: 2.

78. An isolated polynucleotide comprising a sequence of SEQ ID NO: 5.

79. An expression vector comprising the isolated polynucleotide of claim 77 or claim 78.
